(19) United States
(12) Patent Application Publication
(10) Pub. No.: US 2025/0255882 A1
(43) Pub. Date: Aug. 14, 2025

(54) TREATMENT OF CARDIAC CONDITION RELATED COGNITIVE DYSFUNCTION

(71) Applicant: The Trustees of Columbia University in the City of New York, New York, NY (US)

(72) Inventors: Andrew Robert MARKS, New York, NY (US); Haikel DRIDI, New York, NY (US); Steven REIKEN, White Plains, NY (US)

(21) Appl. No.: 19/054,672

(22) Filed: Feb. 14, 2025

Related U.S. Application Data
(60) Provisional application No. 63/553,284, filed on Feb. 14, 2024.

(51) Int. Cl. A61K 31/554; A61K 31/675; A61P 25/28

(52) U.S. Cl. CPC A61K 31/554; A61K 31/675; A61P 25/28

(57) ABSTRACT

A method for treating, preventing or reducing the likelihood of developing cognitive dysfunction that is associated with or induced by a cardiac condition (e.g., heart failure) by administering a therapeutically effective amount of a calcium channel stabilizer to a subject in need thereof. The calcium channel stabilizers include a 1,4-benzothiazepine moiety, including that of the general structural formula:

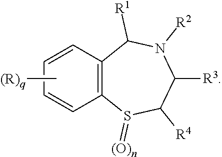

### CROSS-REFERENCE TO RELATED APPLICATIONS

This application claims the benefit U.S. provisional application No. 63/553,284 filed Feb. 14, 2024, the entire content of which is expressly incorporated herein by reference thereto.

### STATEMENT AS TO FEDERALLY SPONSORED RESEARCH

The present disclosure was made with government support under HL145473, DK118240, HL142903, HL140934, AR070194, HL156002 and HL120826 awarded by the National Institutes of Health (NIH). The government has certain rights in the disclosure.

### BACKGROUND

Heart failure (HF) is the most rapidly growing cardiovascular disorder affecting millions worldwide, with associated high rates of mortality, poor quality of life, and high health care costs due to decreased cardiac function and dysfunction of other organ systems. Recent studies suggest that cognitive dysfunction (CD) in HF, known as “cardiogenic dementia” may be caused by HF itself, with a prevalence of 20-80%. CD in HF adversely affects treatment compliance and quality of life.

### SUMMARY OF THE INVENTION

The present disclosure provides a method for treating, preventing, or reducing the likelihood of developing cognitive dysfunction associated with or induced by a cardiac condition. This method comprises administering a therapeutically-effective amount of a Ryanodine Receptor (RyR) calcium channel stabilizer to a subject in need thereof. An exemplary RyR calcium channel stabilizer comprises a 1,4-benzothiazepine moiety.

It has now been found that the hyper-adrenergic state and the enhanced inflammatory response in HF caused neuronal RyR2-mediated intracellular Ca²⁺ leak that subsequently affected cognition and memory. Neuronal Ca²⁺ dyshomeostasis overload and mitochondrial Ca²⁺ content, contributing to oxidative overload, altered the expression of key genes involved in cognitive function. Thus, there is a need to counteract this altered gene expression in order to treat or at least prevent or reduce deterioration of cognitive function after a heart failure episode.

In some embodiments, the cardiac condition is heart failure. In some embodiments, the heart failure is chronic heart failure, acute heart failure, congestive heart failure with reduced ejection fraction, heart failure with preserved ejection fraction, acute decompensated heart failure, systolic heart failure, or diastolic heart failure. In some embodiments, the cardiac condition is characterized by an irregular heartbeat or an arrhythmia. In some embodiments, the subject is a heart failure patient having an implantable cardioverter-defibrillator, wherein the implantable cardioverter-defibrillator is implanted in the patient. In some embodiments, the cardiac condition is myocardial infarction. In some embodiments, the cardiac condition comprises cardiac ischemia/reperfusion injury.

In some embodiments, the cognitive dysfunction results from a RyR2-mediated intracellular Ca²⁺ leak. In some embodiments, the present method of treatment can decrease calcium leak in a RyR2 channel of the subject. Such treatment can also increase RyR2-Castabin2 binding in the subject. The treatment can also decrease open probability (Po) of the RyR2 channel protein in the subject. All of these effects act to at least partially offset cognitive dysfunctions such as a deficit in attention, executive functioning, language, processing speed, learning, short term memory, long term memory, and any combination thereof.

In some embodiments, a compound that can be used in the present methods include those of the general structural formula:

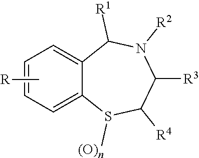

wherein:
   n is 0, 1, or 2 (it is understood by a person of skill in the art that when n is 1 or 2, the bond between sulfur and oxygen can be a double bond);
   R is located at one or more positions on the benzene ring; each R is independently selected from the group consisting of H, halogen, —OH, —NH₂, —NO₂, —CN, —N₃, —SO₃H, acyl, alkyl, alkoxyl, alkylamino, cycloalkyl, heterocyclyl, heterocyclylalkyl, alkenyl, (hetero-)aryl, (hetero-)arylthio, and (hetero-)arylamino; wherein each acyl, alkyl, alkoxyl, alkylamino, cycloalkyl, heterocyclyl, heterocyclylalkyl, alkenyl, (hetero-)aryl, (hetero-)arylthio, and (hetero-)arylamino may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R¹ is selected from the group consisting of H, oxo, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl;
   R² is selected from the group consisting of —C═O(R⁵), —C═S(R⁶), —SO₂R₇, —POR⁸R⁹, —(CH₂)ₘ—R¹⁰, alkyl, aryl, heteroaryl, cycloalkyl, cycloalkylalkyl, and heterocyclyl; wherein each alkyl, aryl, heteroaryl, cycloalkyl, cycloalkylalkyl, and heterocyclyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R³ is selected from the group consisting of H, —CO₂Y, —CONY, acyl, alkyl, alkenyl, aryl, cycloalkyl, and heterocyclyl; wherein each acyl, alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl; and wherein Y is selected from the group consisting of H, alkyl, aryl, cycloalkyl, and heterocyclyl;
   R⁴ is selected from the group consisting of H, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R⁵ is selected from the group consisting of —NR¹⁶, NHNHR¹⁶, NHOH, —OR¹⁵, —CONH₂NHR¹⁶, —CO₂R¹⁵, CONR¹⁶, —CH₂X, acyl, alkenyl, aryl, cycloalkyl, cycloalkylalkyl, heterocyclyl, and heterocyclylalkyl; wherein each acyl, alkenyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R⁶ is selected from the group consisting of —OR₁₅, —NHNR¹⁶, —NHOH, —NR¹⁶, —CH₂X, acyl, alkenyl, alkyl, aryl, cycloalkyl, cycloalkylalkyl, heterocyclyl, and heterocyclylalkyl; wherein each acyl, alkenyl, alkyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R⁷ is selected from the group consisting of —OR¹⁵, —NR¹⁶, —NHNHR¹⁶, —NHOH, —CH₂X, alkyl, aryl, cycloalkyl, cycloalkylalkyl, heterocyclyl, and heterocyclylalkyl; wherein each alkyl, aryl, cycloalkyl, cycloalkylalkyl, heterocyclyl, and heterocyclylalkyl may be substituted with one or more radicals independently selected from the group consisting of halogen, —N, —O, —S, —CN, —Na, nitro, oxo, acyl, alkyl, alkoxyl, alkylamino, alkenyl, aryl, (hetero-)cycloalkyl, and (hetero-)cyclyl;
   R⁸ and R⁹ independently are selected from the group consisting of —OH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R¹⁰ is selected from the group consisting of —NH₂, —OH, —SO₂R¹¹, —NHSO₂R¹¹, —C—O(R¹²), —NHC—O(R¹²), —OC—O(R¹²), and —POR¹³R¹⁴;
   R¹¹, R¹², R¹³, and R¹⁴ independently are selected from the group consisting of H, —OH, —NH₂, —NHNH₂, —NHOH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
   X is selected from the group consisting of halogen, —CN, —CO₂R¹⁵, —CONR¹⁶, —NR¹⁶, —OR¹⁵, —SO₂R⁷, and —POR⁸R⁹; and
   R¹⁵ and R¹⁶ independently are selected from the group consisting of H, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
or a pharmaceutically-acceptable salt, hydrate, solvate, complex, or prodrug thereof.

In some embodiments, compound that can be used in the present methods include those of the general structural formula:

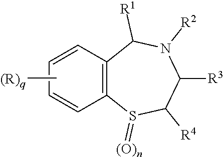

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or —OH;
   each R¹⁰ is —NR¹⁵R¹⁶, —OH, —SO₂R¹¹, —NHSO₂R¹¹, —C(═O) (R¹²), NHC═O(R¹²), —OC—O(R¹²), or —P(═O)R¹³R¹⁴,
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, —OH, —NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, —OH, —NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4,
or a pharmaceutically-acceptable salt, hydrate, solvate, complex, or prodrug thereof.

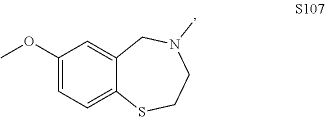

In some embodiments, the compound is represented by the structure or a pharmaceutically-acceptable salt thereof.

In some embodiments, the compounds can penetrate the blood brain barrier so that the treatment can be made in the precise location where the cognitive dysfunction is occurring.

The calcium channel stabilizer can be administered to the subject, for example in a pharmaceutical composition that further comprises at least one pharmaceutically-acceptable excipient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various embodiments will now be described in detail with reference to the accompanying drawings, wherein:

FIGS. 1A-1F illustrate how hippocampal RyR2 channels are remodeled and leaky in patients with heart failure. FIGS. 1A and 1B are representative SDS-PAGE analysis and quantification of modified RyR2 and calstabin2 immunoprecipitated from hippocampi of controls and individuals with HF (IP RyR2; bands normalized to total RyR2). Control (CTRL), n=4; HF n=9. FIG. 1C shows single-channel recordings of RyR2 incorporated in planar lipid bilayers with 150 nM Ca²⁺ in the cis chamber, corresponding to representative experiments performed using human hippocampal samples from controls and HF patients (two traces from two different controls and individuals with HF are shown). FIG. 1D shows Po, To and Tc of RyR2 channels in in controls (n=5) and HF (n=9) hippocampi. FIG. 1E shows ER Ca²⁺ leak measured in microsomes from control (n=4) and HF participant (n=9) hippocampi, and FIG. 1F shows the quantification of microsomal Ca²⁺ leak as the percentage of uptake in controls (n=5) and HF individuals (n=9). Individual values are shown with the mean±s.e.m. (t-test *P<0.05, controls versus HF individuals) and with data derived from biologically independent samples: all statistical tests being two sided (a.u., arbitrary units).

FIGS. 2A-2G illustrate a mouse model of heart failure (myocardial infarction) that is associated with cognitive dysfunction. FIG. 2A compares open field test of mice operated SHAM (n=13), MI (n=22), MI treated with the Rycal® compound ARM036 (MI+ARM036, n=23), MI treated with S107 (MI+S107, n=24), MI treated with propranolol (MI+propranolol, n=10) and MI treated with TGF-β inhibitor (MI+SD-208, n=18) and shows ratios of total time spent in the center area versus periphery area within first 3 min and second 3 min. FIG. 2b shows results of an EPM test showing the ratios of time spent in the open-arm versus closed-arm in SHAM (n=14), MI (n=22), MI+ARM036 (n=18), MI+S107 (n=23), MI+propranolol (n=10) and MI+SD-208 (n=18) mice. FIG. 2C shows results of a novel object recognition test showing the discrimination index in SHAM (n=12), MI (n=21), MI+ARM036 (n=23), MI+S107 (n=22), MI+propranolol (n=10) and MI+SD-208 (n=17) mice. FIG. 2D shows MWM test (learning curves for 4 d) in SHAM (n=22), MI (n=20), MI+ARM036 (n=19), MI+S107 (n=19), MI+propranolol (n=14) and MI+SD-208 (n=19) mice. FIG. 2E shows results of probe trials after escape platform removed showing the total duration spent in the target quadrant at day 5 in SHAM (n=22), MI (n=20), MI+ARM036 (n=18), MI+S107 (n=17), MI+propranolol (n=14) and MI+SD-208 (n=17) mice. FIG. 2F shows the number of target crossings at day 5 in SHAM (n=20), MI (n=20), MI+ARM036 (n=18), MI+S107 (n=16), MI+propranolol (n=14) and MI+SD-208 (n=17) mice. FIG. 2G shows heat maps showing the latency for each group of FIGS. 2A-2F at day 2 and day 4, with individual values shown with mean #s.e.m, wherein the two-tailed t-test *P<0.05 of FIG. 2A shows significance between the first 3 min and second 3 min of each group. One-way analysis of variance (ANOVA) was used to compare the difference between the six groups in FIGS. 2B, 2C, 2E and 2F. Tukey's test was used for multiple comparisons; two-way ANOVA was used in FIG. 2D. Tukey's test post hoc correction for multiple comparisons was used. * P<0.05, SHAM versus MI or MI+ARM036; #P<0.05, MI versus MI+S107, MI+propranolol or MI+SD-208. All statistical tests were two sided with the data derived from biologically independent samples.

FIGS. 3A-3G illustrate a mouse model of heart failure that is associated with leaky hippocampal RyR2. FIG. 3A is a cryogenic electron microscopy structure of RyR2 (gray, top and side view) showing the location of the Ser2808 in the RY3&4 phosphorylation domain (magenta) and calstabin2 (cyan). RyR2 PKA phosphorylation shifted the channel toward a primed state (yellow) 50. FIGS. 3B and 3C are representative SDS-PAGE analysis and quantification of modified RyR2 and calstabin2 immunoprecipitated from hippocampal RyR2 complex (IP RyR2; bands normalized to total RyR2) in SHAM (n=6), MI (n=6), MI+ARM036 (n=6), MI+S107 (n=6), MI+propranolol (n=4) and MI+SD-208 (n=4) mice.

FIG. 3D includes graphs of single-channel traces of RyR2 incorporated in planar lipid bilayers with 150 nM Ca²⁺ in the cis chamber, corresponding to representative experiments performed with hippocampal samples from SHAM (n=6), MI (n=5), MI+ARM036 (n=6), MI+S107 (n=5), MI+propranolol (n=5) and MI+SD-208 (n=5) mice. FIG. 3E are graphs of RyR2 Po, To and Tc in the same groups as FIG. 3D. FIG. 3F is a graph of Ca²⁺ leak measured in microsomes from mouse hippocampi of the same groups as FIG. 3D. FIG. 3G is a bar graph representing the quantification of Ca²⁺ leak as the percentage of uptake in SHAM (n=6), MI (n=6), MI+ARM036 (n=6), MI+S107 (n=6), MI+propranolol (n=3) and MI+SD-208 (n=3) mice. Individual values are shown with the mean±s.e.m. One-way ANOVA and Tukey's test post hoc correction for multiple comparisons shows *P<0.05, SHAM versus MI or MI+ARM036; #P<0.05, MI versus MI+S107, MI+propranolol or MI+SD-208. Data are derived from biologically independent samples. All statistical tests were two sided.

FIGS. 4A-4J illustrate a mouse model of heart failure exhibits impaired long-term potentiation and diminished hippocampal glucose uptake. FIG. 4A is a schematic representation of a hippocampal brain slice for LTP experiments and the positioning of the stimulating and recording electrodes. FIG. 4B is a graph of fEPSPs in hippocampal slices from each experimental group (SHAM (n=13), MI (n=12), MI+ARM036 (n=12), MI+S107 (n=11), MI+propranolol (n=17) and MI+SD-208 (n=16). FIG. 4C is a graph of fEPSPs at 150 min in all the experimental groups. FIG. 4D shows basal neurotransmission (fEPSP slope), which remained unaltered between the different groups. FIG. 4E shows representative microPET images of FDG uptake (percentage of injected dose per gram (% ID/g)) in the mouse brains of different groups. FIG. 4F is a graph of quantification of FDG uptake in the brains of mice from different experimental groups shown as a percentage of the FDG uptake in the SHAM mice (SHAM (n=17), MI (n=6), MI+ARM036 (n=9), MI+S107 (n=6), MI+propranolol (n=7) and MI+SD-208 (n=6)). FIG. 4G is a graph of quantification of 2-min dynamic microPET scans of MI (n=4) and SHAM (n=4) mice demonstrating similar brain blood flow FDG uptake in the brains of both groups of mice during the first 2 min after intravenous injection (% ID/g).

FIGS. 4H-4J are bar graphs of pH, PO2 and PCO2 blood levels in SHAM (n=6) and MI (n=7) mice. Individual values are shown with the mean±s.e.m. One-way ANOVA and Tukey's test post hoc correction for multiple comparisons, *P<0.05, SHAM versus MI or MI+ARM036; #P<0.05, MI versus MI+S107, MI+propranolol or MI+SD-208. A t-test was used in FIGS. 4H-4J. Data are derived from biologically independent samples. All statistical tests were two sided.

FIGS. 5A and 5B illustrate that an adrenergic agonist and RyR2 Ser2808 phospho-mimetic mutation deplete endoplasmic reticulum Ca²⁺ stores in primary hippocampal neurons. FIG. 5A presents representative images of 14-d cultured hippocampal neurons stimulated with 10 mM caffeine. For each condition, the Ca²⁺ levels are shown at baseline, during stimulation and at recovery. FIG. 5B are images showing quantification of caffeine-induced Ca²⁺ release (F/F0) in response to 10 mM caffeine in neurons from wild-type (WT; n=50), WT+S107 (n=30), WT+isoproterenol (ISO; n=26), WT+ISO+propranolol (n=35), neurons expressing RyR2-p.Ser2808Asp untreated (S2808D; n=70) and treated with S107 (p.Ser2808Asp+S107; n=54). Individual values are shown with the mean±s.e.m. (t-test *P<0.05). Scale bar, 10 μm. A reduction in caffeine-induced Ca²⁺ release indicates a Ca²⁺ depleted ER due to persistent RyR2-mediated Ca²⁺ leak. Data are derived from biologically independent samples. All statistical tests were two sided.

FIGS. 6A-6F present a quantitative proteomics analysis of the disclosure. FIG. 6A is graph showing the results of quantitative proteomics performed on hippocampus samples from SHAM (n=4) and MI (n=4) mice. The volcano plot shows differentially expressed proteins (P adjusted<0.05, fold change≥1.5) in SHAM and MI mice. Red indicates upregulation, while blue represents downregulation of protein expression. Black indicates unchanged expression levels. FIG. 6B is a heat map of significantly dysregulated proteins (312 downregulated; 425 upregulated). The color scale bar shows the row normalized log 2 protein abundance. FIGS. 6C-6F are dot plots that show top ten GO biological processes (FIG. 6C), molecular functions (FIG. 6D), cellular components (FIG. 6E) and KEGG pathways (FIG. 6F) that were enriched from differentially expressed proteins. Significantly changed protein abundance was determined by unpaired t-test with a threshold for significance of P<0.05 (permutation-based FDR correction), fold change≥1.5, unique peptides≥2. Data are derived from biologically independent samples. All statistical tests were two sided.

FIGS. 7A-7E illustrate altered synaptic protein expression in heart failure. FIG. 7A is a cohort plot representation of differentially expressed synaptic proteins (SHAM versus MI) from six significantly enriched synaptic transmission GO terms and generated by GOplot. The color map represents fold change of proteins (log 2 scale). Selected proteins in the SNARE pathway are highlighted in red (upregulated) or green (downregulated). FIGS. 7B and 7C are immunoblots showing total expression of SNAP25, VAMP8, SYT2 and CPLX3, normalized to GAPDH in the hippocampi of controls (n=4) and individuals with HF (n=9). Individual values are shown with the mean±s.e.m. (t-test *P<0.05, control versus individuals with HF). FIGS. 7D and 7E are immunoblots showing total expression of SNAP25, VAMP8, SYT2 and CPLX3, normalized to GAPDH in the hippocampi of SHAM (n=6), MI (n=6), MI+ARM036 (n=6), MI+S107 (n=6), MI+propranolol (n=4) and MI+SD-208 (n=4) mice. Individual values are shown with the mean #s.e.m. One-way ANOVA and Tukey's test post hoc correction for multiple comparisons, *P<0.05, SHAM versus MI or MI+ARM036; #P<0.05 MI versus MI+S107, MI+propranolol or MI+SD-208. Data are derived from biologically independent samples. All statistical tests were two sided.

FIG. 8 is a schematic illustration of neuronal Ca²⁺ signaling in heart failure.

FIGS. 9A-9G present a mouse model of leaky RyR2 (constitutive RyR2 PKA-phosphorylation) that is associated with cognitive dysfunction. A mouse model of leaky RyR2 (phospho-mimetic mutation) is associated with cognitive dysfunction. FIG. 9A is a graph of the results of an open field test of SHAM (n=14), S2808A-SHAM (n=8), S2808AMI (n=8), S2808D (n=13), and S2808D+S107 (n=8) mice. Ratios of total time spent in the center area versus periphery area within first (1st) 3 min and second (2nd) 3 min are shown. FIG. 9B is a graph of the results of an elevated plus maze test in SHAM (n=14), S2808ASHAM (n=8), S2808A-MI (n=8), S2808D (n=13), and S2808D+S107 (n=8) mice. Ratios of time spent on the open-arm versus closed-arm are shown. FIG. 9C is a graph of results from a novel object recognition test in SHAM (n=14), S2808A-SHAM (n=8), S2808A-MI (n=8), S2808D (n=13), and S2808D+S107 (n=8) mice. Discrimination index is shown. FIG. 9D is a graph of the results of a Morris water maze (MWM) test (learning curves) in SHAM (n=14), S2808ASHAM (n=8), S2808A-MI (n=8), S2808D (n=13), and S2808D+S107 (n=8) mice. FIG. 9E is a graph of probe trials after escape platform was removed in the same groups showing the total duration spent in the target quadrant. FIG. 9F is a graph of a number of target crossings SHAM (n=14), S2808A-SHAM (n=8), S2808A-MI (n=8), S2808D (n=13), and S2808D+S107 (n=8) mice. FIG. 9G is a heat map showing the latency from each group at Day 2 and Day 4. Individual values are shown with mean±SEM (t-test * p<0.05 in panel A shows significance between the first 3 min and second 3 min of the same groups. One-way ANOVA was used to compare the difference between the 5 groups in panel B, C, E and F; Two-way ANNOVA was used in panel D. Tukey's test post-hoc correction for multiple comparisons was used; * p<0.05, S2808ASHAM vs. S2808D or S2808D+S107; #p<0.05, S2808D vs. S2808D+S107. No differences were detected between S2808A-SHAM and S2808A-MI. All statistical tests were two-sided. Data are derived from biologically independent samples.

FIGS. 10A-10G illustrate cognitive function in RyR1-S2844D mice. FIG. 10A is a graph showing the results of open field test using WT mice (n=10) and a mouse model with leaky RyR1 channels (S2844D) (n=21). Ratios of total time spent in the center area versus periphery area within first 3 min and second 3 min are shown. FIG. 10B is a graph showing the results of elevated plus maze test in WT mice (n=10) and S2808D (n=21). Ratios of time spent in the open-arm versus closed-arm are shown. FIG. 10C is a graph showing the results of a novel object recognition test in WT mice (n=10) and S2808D (n=21). Discrimination index is shown. FIG. 10D is a graph showing the results of a MWM test (learning curves) in WT mice (n=10) and S2808D (n=21). FIG. 10E is a graph showing the results of probe trials after escape platform removed in the same groups showing the total duration spent in the target quadrant in WT mice (n=10) and S2808D (n=21). FIG. 10F is a graph showing the results of number of target crossings in WT mice (n=10) and S2808D (n=21). FIG. 10G is a heat map showing the latency from each group at Day 2 and Day 5. Individual values are shown with mean±SEM. T-test was used in panel A-C, E-F, * p<0.05 in panel A shows significance between the first 3 min and second 3 min of each group). Two-way ANOVA was used in panel D. Tukey's test post-hoc correction for multiple comparisons was used. All statistical tests were two-sided. Data are derived from biologically independent samples.

FIGS. 11A-11H illustrate constitutive RyR2 phosphorylation on Ser2808 (S2808D mice) induces ER Ca²⁺ leak in the hippocampus. Phospho-mimetic mutation (RyR2-S2808D mice) induces ER Ca²⁺ leak in the hippocampus. FIGS. 11A and 11B are representative SDS-PAGE analysis and quantification of modified RyR2 and calstabin2 immunoprecipitated from hippocampus of S2808A-SHAM (n=4), S2808A-MI (n=4), S2808D (n=4), S2808D+S107 mice (n=4) (IP RyR2: Bands normalized to total RyR2); n=4 in each group. FIG. 11C is a graph showing the results of an ER Ca²⁺ leak measured in microsomes from hippocampi of S2808A-SHAM (n=4), S2808A-MI (n=4), S2808D, S2808D+S107 mice (n=4). FIG. 11D includes bar graphs that represent the quantification of Ca²⁺ leak as the percentage of uptake in all the experimental groups (n=4 per group). FIG. 11E is a graph showing the results of single-channel traces of RyR2 incorporated in planar lipid bilayers with 150 nM Ca²⁺ in the cis chamber, corresponding to representative experiments performed with hippocampal samples from S2808A-SHAM, S2808A-MI, S2808D, S2808D+S107 mice. FIGS. 11F, 11G and 11H are graphs showing the results of RyR2 open probability (Po), mean open time (To), and mean close time (Tc) in S2808A-SHAM, S2808A-MI, S2808D, and S2808D+S107 mice (n=n=5, 5, 4 and 4 respectively). Individual values are shown with mean±SEM. One way-ANOVA and Tukey's test post-hoc correction for multiple comparisons shows * p<0.05, S2808A-SHAM vs. S2808D or S2808D+S107; #p<0.05, S2808D vs. S2808D+S107. No differences were detected between S2808A-SHAM and S2808A-MI. All statistical tests were two-sided. Data are derived from biologically independent samples.

FIGS. 12A-12D illustrate TGF-β activation in HF. FIG. 12A includes immunoblots showing expressing levels of TGF-β, phosphorylated SMAD3, total SMAD3, and NOX2 binding to RyR2 in the hippocampi of controls (n=4) and HF patients (n=9). FIG. 12B includes bar graphs depicting the ratio of TGF-β expression normalized to GAPDH, phosphorylated SMAD3 to total SMAD3 and NOX2 binding to RyR2 (IP RyR2). The same quantity of proteins was loaded on two separate gels and blotted separately for SMAD3 and pSMAD3. Individual values are shown with mean±SEM (t-test * p<0.05, Controls vs. HF patients). FIG. 12C includes immunoblots showing expressing levels of TGF-β, phosphorylated SMAD3, total SMAD3, and NOX2 binding to RyR2 in the hippocampi of SHAM, MI, MI+ARM036, MI+S107, MI+propranolol and MI+SD-208 mice (n=6, 6, 6, 6, 4 and 4 respectively). FIG. 12D includes bar graphs depicting the ratio of TGF-β expression normalized to GAPDH, phosphorylated SMAD3 to total SMAD3 and NOX2 binding to RyR2 (IP RyR2). The same quantity of proteins was loaded on two separate gels and blotted separately for SMAD3 and pSMAD3. Individual values are shown with mean±SEM. One-way ANOVA and Tukey's test post-hoc correction for multiple comparisons shows * p<0.05, SHAM vs. MI, MI+ARM036 or MI+S107; #p<0.05, MI vs. MI+S107, MI+propranolol or MI+SD-208. All statistical tests were two-sided. Data are derived from biologically independent samples.

FIGS. 13A-13C include dot plots that illustrate pre-ranked gene set enrichment analysis (GSEA) of the hippocampal proteomics. FIG. 13A shows top 20 up- and top 20 downregulated GO biological process. FIG. 13B shows top 10 up- and top 20 down-regulated GO cellular component. FIG. 13C shows top 10 up-regulated and top 20 down-regulated GO molecular function terms. Significantly changed protein abundance was determined by unpaired t-test with a threshold for significance of p<0.05 (permutation-based FDR correction), fold-change≥1.5, unique peptides≥2. Data are derived from biologically independent samples. All statistical tests were two-sided. Source file PRIDE #PXD042295.

FIGS. 14A-14D show gene set enrichment analysis (GSEA) of the hippocampal proteomics. This figure presents enrichment plots of representative KEGG pathway gene sets demonstrate that oxidative phosphorylation (FIG. 14A), Parkinson's disease (FIG. 14B), Alzheimer's disease (FIG. 14C), and Huntington's disease (FIG. 14D) are significantly enriched in MI compared to SHAM. The heatmap on the right side of each panel visualizes the genes contributing to the enriched pathways. Signal-to-noise ratio was used to rank the genes per their correlation with either MI phenotype (red) or SHAM phenotype (blue). The y-axis represents enrichment score (ES) and on the x-axis are genes (vertical black lines) represented in gene sets. The GSEA analysis calculates an enrichment score (the maximum deviation from zero) reflecting the degree of over-representation of a gene set at the top or the bottom of the ranked gene list. A positive ES indicates gene set enrichment at the top of the ranked list; a negative ES indicates gene set enrichment at the bottom of the ranked list. NES, normalized enrichment score; FDR, FDR adjusted p-value.

FIGS. 15A-15F present the results of an exemplary RNA sequencing analysis. RNA sequencing was performed on the hippocampi of SHAM and MI mice (n=4 for each group). FIG. 15A is a volcano plot that shows differentially expressed genes (p-adj<0.05, fold-change≥1.3) in SHAM and MI mice. Red indicates up-regulated, while blue represents down-regulated genes. Black indicates unchanged expression levels. FIG. 15B is a heat map that shows significantly dysregulated genes (down-regulated: 2003, upregulated: 1149 genes), the color scale bar shows the row normalized log 2 protein abundance. FIG. 15C includes dot plots that show top 10 GO biological processes, FIG. 15D showing molecular functions, FIG. 15E showing cellular components, and FIG. 15F showing KEGG pathways that were enriched from differentially expressed genes. Significantly changed gene abundance was determined by unpaired t-test with a threshold for significance of p<0.05 (permutation-based FDR correction), fold-change≥1.5. Data are derived from biologically independent samples. All statistical tests were two-sided.

FIGS. 16A-16C include dot plots that show a pre-ranked gene set enrichment analysis (GSEA) of the RNA sequencing of FIGS. 15A-15F. FIG. 16A shows top 20 up- and top 20 down-regulated GO biological process. FIG. 16B shows top 20 up-regulated and top 20 down-regulated GO cellular component. FIG. 16C shows top 20 up-regulated and top 20 down-regulated GO molecular function terms. Significantly changed gene abundance was determined by unpaired t-test with a threshold for significance of p<0.05 (permutation-based FDR correction), fold-change≥1.5. Data are derived from biologically independent samples. All statistical tests were two-sided.

FIGS. 17A-17D illustrate the results of a gene set enrichment analysis (GSEA) of the hippocampal RNA sequencing in the form of enrichment plots of representative KEGG pathway gene sets that demonstrate that oxidative phosphorylation (FIG. 17A), Parkinson's disease (FIG. 17B), Alzheimer's disease (FIG. 17C), and Huntington's disease (FIG. 17D) are significantly enriched in MI compared to SHAM. The heatmap on the right side of each panel visualizes the genes contributing to the enriched pathways. Signal-to-Noise ratio was used to rank the genes per their correlation with either MI phenotype (red) or SHAM phenotype (blue). The y-axis represents enrichment score (ES) and on the x-axis are genes (vertical black lines) represented in gene sets. The GSEA analysis calculates an enrichment score (the maximum deviation from zero) reflecting the degree of over-representation of a gene set at the top or the bottom of the ranked gene list. A positive ES indicates gene set enrichment at the top of the ranked list; a negative ES indicates gene set enrichment at the bottom of the ranked list. NES, normalized enrichment score; FDR, FDR adjusted p-value.

FIGS. 18A-18C illustrate mitochondrial Ca²⁺ overload and oxidative stress in HF. FIG. 18A is a cohort plot representation of differentially expressed mitochondrial proteins (SHAM vs MI) from 4 significantly enriched mitochondrial GO-terms and generated by Goplot. The color map represents fold change of proteins (log 2 scale); FIG. 18B is a bar graph showing Ca²⁺ accumulation in isolated mitochondria from SHAM (n=6), MI (n=5), MI+ARM036 (n=5), and MI+S107 (n=5) mice; and FIG. 18C is a bar graph showing reactive oxygen species (ROS) production in isolated mitochondria from SHAM (n=6), MI (n=6), MI+ARM036 (n=6), and MI+S107 (n=5) mice. Individual values are shown with mean±SEM (one-way ANOVA and Tukey's test post-hoc correction for multiple comparisons show * p<0.05, SHAM vs. MI or MI+ARM036; #p<0.05, MI vs. MI+S107). All statistical tests were two-sided.

### DETAILED DESCRIPTION

It has now been found that RyR2, an intracellular calcium release channel within hippocampal neurons, is subject to post-translational modifications (PTM) in individuals with heart failure (HF). PTMs can occur as a result of increased adrenergic signaling and activation of the transforming growth factor-beta pathway. As a consequence, neuronal RyR2 became leaky along with modified cardiac RyR2. In a mouse model of heart failure, the neuronal calcium leak induced protein expression changes that lead to cognitive dysfunction.

Hippocampal neurons from individuals and mice with HF show that the RyR2/intracellular Ca²⁺ release channels were subjected to post-translational modification (PTM) and were leaky. RyR2 PTM included protein kinase A phosphorylation, oxidation, nitrosylation and depletion of the stabilizing subunit calstabin2. RyR2 PTM was caused by hyper-adrenergic signaling and activation of the transforming growth factor-beta pathway. HF mice treated with a RyR2 stabilizer drug (S107), beta blocker (propranolol) or transforming growth factor-beta inhibitor (SD-208), or genetically engineered mice resistant to RyR2 Ca²⁺ leak (RyR2-p.Ser2808Ala), were protected against HF-induced CD. Taken together, this suggests that HF is a systemic illness driven by intracellular Ca²⁺ leak that includes cardiogenic dementia.

It has now been found that heart failure-induced cognitive dysfunction can be mediated by blocking and/or resolving intracellular Ca²⁺ leaking through ryanodine receptor type 2. Accordingly, stabilizing leaky RyR2 channels using a small-molecule Rycal® drug e.g., S107, was found to reduce or prevent cognitive impairment induced by HF. In particular, treatment with a brain penetrant Rycal® drug was able to restore normal calcium handling in mouse neurons and to alleviate cardiogenic dementia effects in mice. This finding sheds a new light on heart failure-induced cognitive dysfunction and the causal role of RyR2-related intracellular calcium signaling in the brain. These findings expand the role of RyR2 in heart failure beyond its previously established cardiac effects.

In some embodiments, the present disclosure provides a method of treating cognitive dysfunction associated with or induced by a cardiac condition, the method comprising administering a therapeutically-effective amount of a calcium channel stabilizer to a subject in need thereof, wherein the calcium channel stabilizer is represented by the structure:

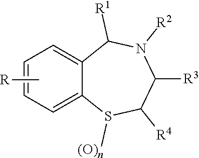

wherein,
   n is 0, 1, or 2;
   R is located at one or more positions on the benzene ring; each R is independently selected from the group consisting of H, halogen, —OH, —NH₂, —NO₂, —CN, —N₃, —SO₃H, acyl, alkyl, alkoxyl, alkylamino, cycloalkyl, heterocyclyl, heterocyclylalkyl, alkenyl, (hetero-)aryl, (hetero-)arylthio, and (hetero-)arylamino; wherein each acyl, alkyl, alkoxyl, alkylamino, cycloalkyl, heterocyclyl, heterocyclylalkyl, alkenyl, (hetero-)aryl, (hetero-)arylthio, and (hetero-)arylamino may be substituted with one or more radicals independently selected from the group consisting of halogen, —N, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R¹ is selected from the group consisting of H, oxo, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl;
   R² is selected from the group consisting of —C═O(R⁵), —C═S(R⁶), —SO₂R⁷, —POR⁸R⁹, —(CH₂)ₘ—R¹⁰, alkyl, aryl, heteroaryl, cycloalkyl, cycloalkylalkyl, and heterocyclyl; wherein each alkyl, aryl, heteroaryl, cycloalkyl, cycloalkylalkyl, and heterocyclyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R³ is selected from the group consisting of H, —CO₂Y, —CONY, acyl, alkyl, alkenyl, aryl, cycloalkyl, and heterocyclyl; wherein each acyl, alkyl, alkenyl, aryl, cycloalkyl, and heterocyclyl may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl; and wherein Y is selected from the group consisting of H, alkyl, aryl, cycloalkyl, and heterocyclyl;
   R⁴ is selected from the group consisting of H, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl;
   R⁵ is selected from the group consisting of —NR¹⁶, NHNHR¹⁶, NHOH, —OR¹⁵, —CONH₂NHR¹⁶, —CO₂R¹⁵, CONR¹⁶, —CH₂X, acyl, alkenyl, aryl, cycloalkyl, cycloalkylalkyl, heterocyclyl, and heterocyclylalkyl; wherein each acyl, alkenyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R⁶ is selected from the group consisting of —OR¹⁵, —NHNR¹⁶, —NHOH, —NR¹⁶, —CH₂X, acyl, alkenyl, alkyl, aryl, cycloalkyl, cycloalkylalkyl, heterocyclyl, and heterocyclylalkyl; wherein each acyl, alkenyl, alkyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R⁷ is selected from the group consisting of —OR¹⁵, —NR¹⁶, —NHNHR¹⁶, —NHOH, —CH₂X, alkyl, aryl, cycloalkyl, cycloalkylalkyl, heterocyclyl, and heterocyclylalkyl; wherein each alkyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R⁸ and R⁹ independently are selected from the group consisting of OH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
   R¹⁰ is selected from the group consisting of —NH₂, —OH, —SO₂R¹¹, —NHSO₂R¹¹, —C═O(R¹²), —NHC═O(R¹²), —OC—O(R¹²), and —POR¹³R¹⁴.
   R¹¹, R¹², R¹³, and R¹⁴ independently are selected from the group consisting of H, —OH, —NH₂, —NHNH₂, —NHOH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
   X is selected from the group consisting of halogen, —CN, —CO₂R¹⁵, —CONR¹⁶, —NR¹⁶, —OR¹⁵, —SO₂R⁷, and —POR⁸R⁹; and
   R¹⁵ and R¹⁶ independently are selected from the group consisting of H, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
or a pharmaceutically-acceptable salt, hydrate, solvate, complex, or prodrug thereof.

In some embodiments, the present disclosure provides a method of treating cognitive dysfunction associated with or induced by a cardiac condition, the method comprising administering a therapeutically-effective amount of a calcium channel stabilizer to a subject in need thereof, wherein the calcium channel stabilizer is represented by the structure:

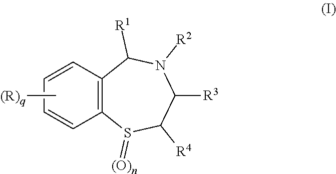

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰,
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂) NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or —OH;
   each R¹⁰ is —NR¹⁵R¹⁶, —OH, —SO₂R¹¹, —NHSO₂R¹¹, —C(═O) (R¹²), —NHC—O(R¹²), OC—O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, —OH, —NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, —OH, —NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   n is 0, 1, or 2;
   q is 0, 1, 2, 3, or 4;
   t is 1, 2, 3, 4, 5, or 6; and
   m is 1, 2, 3, or 4,
or a pharmaceutically-acceptable salt, hydrate, solvate, complex, or prodrug thereof.

### Definitions

The following definitions are provided for the purpose of understanding the present subject matter and for construing the appended patent claims.

It is noted that, as used in this specification and the appended claims, the singular forms “a”, “an”, and “the” include plural references unless the context clearly dictates otherwise.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

As used herein, the term “salt” has the same meaning as commonly understood to one of ordinary skill in the art. Specifically, a salt is a chemical compound consisting of an ionic assembly of positively charged cations and negatively charged anions.

As used herein, the term “hydrate” has the same meaning as commonly understood to one of ordinary skill in the art. Specifically, a hydrate is a compound with extra water molecules that are part of its structure.

As used herein, the term “solvate” has the same meaning as commonly understood to one of ordinary skill in the art. Specifically, a solvate is a compound formed by the interaction of a solvent and a solute.

As used herein, the term “complex” has the same meaning as commonly understood to one of ordinary skill in the art. Specifically, a complex is a molecular entity formed by loose association involving two or more component molecular entities (ionic or uncharged), or the corresponding chemical species. The bonding between the components is normally weaker than in a covalent bond.

As used herein, the term “prodrug” has the same meaning as commonly understood to one of ordinary skill in the art. Specifically, a prodrug is a precursor of a drug—a compound that, on administration to a subject, undergoes metabolic processes that convert the compound to the drug.

As used herein, the term “treating” has the same meaning in the present context as commonly understood to one of ordinary skill in the art. Specifically, “treating” a disease or condition means providing any form of relief to the patient from the disease or condition or its recurrence, including without limitation, reducing severity, reducing expected further development, or reducing the expected duration, of the disease or condition or any symptoms or recurrence thereof, or otherwise providing relief to the patient from normally-expected development, severity, duration, or any lasting consequences of the disease or condition or any of its symptoms.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

### Cognitive Dysfunction in Cardiac Conditions

Cognitive dysfunction (CD) includes forgetfulness and poor learning ability, which may impair self-care and compliance in as many as 90% of those with HF. Noncompliance increases the risk of mortality and morbidity. Indeed, CD impairs the ability of individuals with HF to make decisions in critical situations, such as early recognition and interpretation of worsening symptoms, and making appropriate decisions about their health. People with cardiac conditions (e.g., subjects with heart failure, e.g., heart failure with preserved ejection function) also exhibit CD, including verbal memory and executive function deficits, known as cognitive inflexibility. Structural changes in the brain including atrophy, increased white matter hyper-intensities, gray matter loss and silent cerebral infarction, are frequently observed in HF patients with CD. Interestingly, these structural and functional changes coincide with a chronic inflammatory response and neurohormonal activation including the renin-angiotensin-aldosterone system and the adrenergic pathway. Furthermore, clinical studies have linked cardiovascular diseases, dementia and Alzheimer's disease through common triggers, including inflammation, oxidative stress, hypoxia and adrenergic signaling.

Norepinephrine modulates the levels of consciousness. The sympathetic nervous system is continuously activated in patients with HF and is known to be part of a major upstream signaling pathway that alters intracellular Ca²⁺ homeostasis and tightly controls neuronal cellular function and survival. Ca²⁺ dyshomeostasis is a hallmark of neurodegenerative diseases, including Alzheimer's disease, Huntington's disease and Parkinson's disease. Intracellular Ca²⁺ signaling plays a role in regulating long-term potentiation (LTP), long-term depression and neurodegeneration.

In neurons, activation of inositol-1,4,5-trisphosphate receptors (IP₃Rs) and RyRs amplifies intracellular Ca²⁺ signals. Increased intracellular Ca²⁺ concentration activates Ca²⁺-dependent processes involved in plasticity and synaptic transmission that are required for learning and memory. RyR2, the Ca²⁺-activated intracellular Ca²⁺ release channel on the sarcoplasmic reticulum (SR) or endoplasmic reticulum (ER), is a homotetrameric macromolecular protein complex that includes four RyR2 monomers, 565-kDa polypeptide each. The RyR2 channel is regulated by kinases and phosphatases, phosphodiesterase, calmodulin, and the stabilizing subunit calstabin2 (FKBP12.6). Protein kinase A (PKA) and Ca²⁺/calmodulin-dependent protein kinase II (CAMKII) tether to RyR2 and phosphorylate the channel at Ser2808 and Ser2814, respectively. PKA hyper-phosphorylation and/or oxidation/nitrosylation of RyR2 cause calstabin2 dissociation, leading to leaky channels that do not close properly.

Previous reports disclose that RyR channels are dysfunctional not only in the cardiomyocytes of patients with HF but also in the skeletal muscle, suggesting the existence of a common mechanism that primarily affects RyR2 in the cardiac muscle and propagates to affect RyRs in other organs expressing different isoforms of the channels, such as RyR2 in the pancreatic beta cells (which may cause diabetes) and in the brain (which may impair cognitive function), and RyR1 in the diaphragm/lung (which may cause respiratory disorders) and locomotor muscle (which may cause exercise intolerance and muscle fatigue). Although ryanodine receptor type 2 (RyR2) has been linked to cardiac muscle dysfunction, its role in CD in HF remains unclear.

In some embodiments, the cognitive dysfunction comprises one or more of a deficit in attention, executive functioning, language, processing speed, learning, short term memory, long term memory, verbal memory, auditory memory, and any combination thereof.

### Cardiac Conditions

Subjects treated by the methods of the present disclosure can be a subject diagnosed with a cardiac condition. In some embodiments, the cardiac condition is heart failure.

In some embodiments, the heart failure is chronic heart failure. In some embodiments, the heart failure is acute heart failure. In some embodiments, the heart failure is heart failure with reduced ejection fraction. In some embodiments, the heart failure is heart failure with preserved ejection fraction. In some embodiments, the heart failure is acute decompensated heart failure. In some embodiments, the heart failure is heart failure characterized by systolic dysfunction, or heart failure with diastolic dysfunction.

In some embodiments, the cardiac condition is characterized by an irregular heartbeat or an arrhythmia.

In some embodiments, the subject is a heart failure patient having an implantable cardioverter-defibrillator, wherein the implantable cardioverter-defibrillator is implanted in the patient.

In some embodiments, the cardiac condition is myocardial infarction.

In some embodiments, the cardiac condition comprises cardiac ischemia/reperfusion injury.

### Compounds

The compounds of use in the methods of the present disclosure include any one of a wide variety of calcium channel stabilizers that comprise a 1,4-benzothiazepine moiety.

Calcium channel stabilizer can include compounds commonly referred to as Rycal® compounds, such as 1,4-benzothiazepines and related structures, described in U.S. Pat. No. 8,710,045, issued on Apr. 29, 2014, and U.S. Pat. No. 8,022,058, issued on Sep. 20, 2011, the contents of each of which are incorporated by reference herein.

In some embodiments, a compound capable of binding RyR2 is a compound of Formula I:

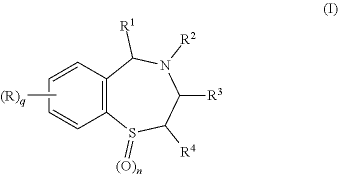

wherein,
   n is 0, 1, or 2;
   q is 0, 1, 2, 3, or 4;
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or —OH;
   each R¹⁰ is —NR¹⁵R¹⁶, —OH, —SO₂R¹¹, —NHSO₂R¹¹, —C(═O)(R¹²), —NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, —OH, —NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
   each R¹⁵ and R¹⁶ is independently acyl, alkenyl, alkoxyl, —OH, —NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   t is 1, 2, 3, 4, 5, or 6;
   m is 1, 2, 3, or 4;
or a pharmaceutically-acceptable salt thereof.

In some embodiments, R² is unsubstituted alkyl.

In some embodiments, the present disclosure provides compounds of Formula I-a:

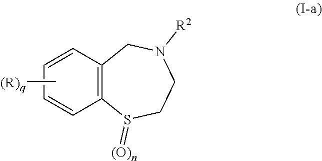

wherein:
   n is 0, 1, or 2;
   q is 0, 1, 2, 3, or 4;
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or —OH;
   each R¹⁰ is —NR¹⁵R¹⁶, —OH, —SO₂R¹¹, —NHSO₂R¹¹, —C(═O)R¹², —NH(C═O)R¹², —O(C═O)R¹², or —P(═O)R¹³R¹⁴; m is 0, 1, 2, 3, or 4;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, —OH, —NH₂, —NHNH₂, or —NHOH;
   each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
   each R¹⁵ and R¹⁶ is independently acyl, alkenyl, alkoxyl, —OH, —NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
   or a pharmaceutically-acceptable salt thereof.

In some embodiments, the present disclosure provides a compound of formula I-a, wherein each R is independently halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1, or 2.

In some embodiments, the present disclosure provides a compound of formula I-a, wherein R₂ is —C═O(R⁵), —C═S(R⁶), —SO₂R⁷, —P(═O)R⁸R⁹, or —(CH₂)ₘ—R¹⁰.

In some embodiments, the present disclosure provides a compound of formula I-b:

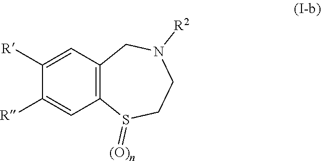

wherein:
   R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰; and
   n is 0, 1, or 2;
or a pharmaceutically-acceptable salt thereof.

In some embodiments, the present disclosure provides a compound of formula I-b, wherein R′ and R″ are each independently H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-b, wherein R₂ is —C═O(R₅), —C═S(R₆), —SO₂R₇, —P(═O)R₈R⁹, or —(CH₂)ₘ—R₁₀.

In some embodiments, the present disclosure provides a compound formula of I-c:

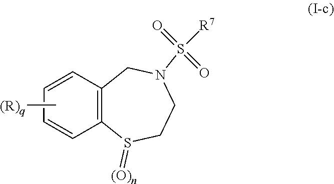

wherein:
   n is 0, 1, or 2;
   q is 0, 1, 2, 3, or 4;
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X; or a pharmaceutically-acceptable salt thereof.

In some embodiments, the present disclosure provides a compound of formula I-c, wherein each R is independently halogen, —OH, —OMe, —NH₂, —NO₂, —CN, —CF₃, —OCF₃, —N₃, —S(═O)C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1, or 2.

In some embodiments, the present disclosure provides a compound of formula I-c, wherein R⁷ is alkyl, alkenyl, aryl, cycloalkyl, cycloalkylalkyl, heterocyclyl, or heterocyclylalkyl, each of which is independently substituted or unsubstituted; or —OH or —NR¹⁵R¹⁶.

In some embodiments, the present disclosure provides a compound of formula of I-d:

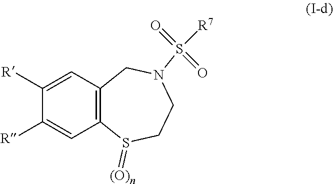

wherein:
   n is 0, 1, or 2;
   R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₃, —NO₂, —CN, —CF₃, —OCF₃, —N₃, —SO₃H, —S(═O)₂alkyl, —S(═O)alkyl, or —OS(═O)₂CF₃;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X,
or a pharmaceutically-acceptable salt thereof.

In some embodiments, the present disclosure provides a compound of formula wherein R′ and R″ are each independently H, halogen, —OH, —OMe, —NH₂, —NO₂, —CN, —CF₃, —OCF₃, —N₃, —S(O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-d, wherein R₇ is alkyl, alkenyl, aryl, cycloalkyl, cycloalkylalkyl, heterocyclyl, or heterocyclylalkyl, each of which is independently substituted or unsubstituted; or —OH, or —NR¹⁵R¹⁶.

In some embodiments, the present disclosure provides a compound of formula of I-e:

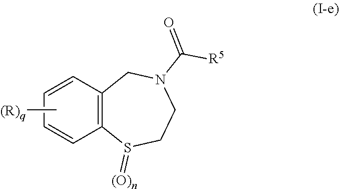

wherein:
   n is 0, 1, or 2;
   q is 0, 1, 2, 3, or 4;
   each R is independently acyl, —O)-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃; and
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group,
or a pharmaceutically-acceptable salt thereof.

In some embodiments, the present disclosure provides a compound of formula I-e, wherein each R is independently halogen, —OH, —OMe, —NH₃, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1, or 2.

In some embodiments, the present disclosure provides a compound of formula I-e, wherein R₅ is alkyl, alkenyl, aryl, cycloalkyl, cycloalkylalkyl, heterocyclyl, or heterocyclylalkyl, each of which is independently substituted or unsubstituted; or —NR¹⁵R¹⁶, —NHOH, —OR¹⁵, or —CH₂X.

In some embodiments, the present disclosure provides a compound of formula of I-f:

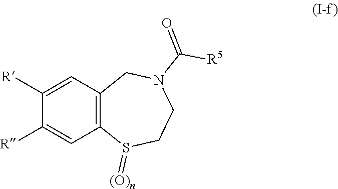

wherein:
   n is 0, 1, or 2;
   R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)alkyl, —S(═O)alkyl, or —OS(═O)₂CF₃;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group,
or a pharmaceutically-acceptable salt thereof.

In some embodiments, the present disclosure provides a compound of formula I-f, wherein R′ and R″ are each independently H, halogen, —OH, —OMe, —NH₂, —NO₂, —CN, —CF₃, —OCF₃, —N₃, —S(═O)₂C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-f, wherein R₅ is alkyl, alkenyl, aryl, cycloalkyl, cycloalkylalkyl, heterocyclyl, or heterocyclylalkyl, each of which is independently substituted or unsubstituted; or —NR¹⁵R¹⁶, —NHOH, —OR″, or —CH₂X.

In some embodiments, the present disclosure provides a compound of formula of I-g:

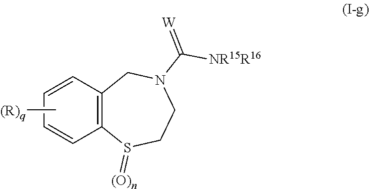

wherein:
   n is 0, 1, or 2;
   q is 0, 1, 2, 3, or 4;
   W is S or O;
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   each R¹⁵ and R¹⁶ is independently acyl, alkenyl, alkoxyl, —OH, —NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded may form a heterocycle that is substituted or unsubstituted, or a pharmaceutically-acceptable salt thereof.

In some embodiments, the present disclosure provides a compound of formula I-g, wherein each R is independently selected from the group consisting of H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl and propenyl; and n is 0, 1, or 2.

In some embodiments, the present disclosure provides a compound of formula I-g, wherein R¹⁵ and R¹⁶ are each independently alkyl, alkylamino, aryl, cycloalkyl, cycloalkylalkyl, heterocyclyl, and heterocyclylalkyl, each of which is independently substituted or unsubstituted; or H, —OH, or —NH₂; or R¹⁵ and R¹⁶ together with the N to which they are bonded form a heterocycle that is substituted or unsubstituted.

In some embodiments, the present disclosure provides a compound of formula I-g, wherein W is O or S.

In some embodiments, the present disclosure provides a compound of formula of I-h:

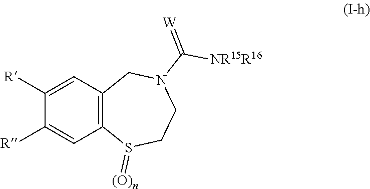

n is 0, 1, or 2;
W is S or O;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃,
or a pharmaceutically-acceptable salt thereof.

In some embodiments, the present disclosure provides a compound of formula wherein R′ and R″ are each independently H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-h, wherein R¹⁵ and R¹⁶ are each independently alkyl, alkylamino, aryl, cycloalkyl, cycloalkylalkyl, heterocyclyl, or heterocyclylalkyl, each of which is independently substituted or unsubstituted; or H, —OH, —NH₂; or R¹⁵ and R¹⁶ together with the N to which R¹⁵ and R¹⁶ are bonded form a heterocycle that is substituted or unsubstituted.

In some embodiments, the present disclosure provides a compound of formula I-g, wherein W is O or S.

In some embodiments, the present disclosure provides a compound of formula of I-i:

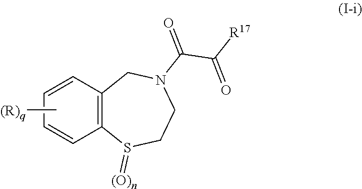

wherein
   R¹⁷ is alkenyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —NHOH, —OR¹⁵, or —CH₂X;
   n is 0, 1, or 2;
   q is 0, 1, 2, 3, or 4; and
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃,
or a pharmaceutically-acceptable salt thereof.

In some embodiments, the present disclosure provides a compound of formula I-i, wherein each R is independently wS halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1, or 2.

In some embodiments, the present disclosure provides a compound of formula I-i, wherein R¹⁷ is —NR¹⁵R¹⁶ or —OR¹⁵. In some embodiments, R¹⁷ is —OH, —OMe, —NHEt, —NHPh, —NH₂, or —NHCH₂pyridyl.

In some embodiments, the present disclosure provides a compound of formula of I-j:

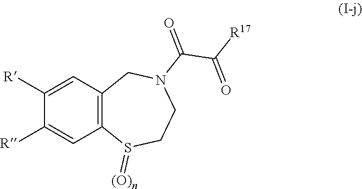

wherein:
   R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃; R¹⁷ is selected from the group consisting of —NR¹⁵R¹⁶, —NHOH, —OR¹⁵, —CH₂X, alkenyl, aryl, cycloalkyl, cycloalkylalkyl, heterocyclyl, and heterocyclylalkyl; wherein each alkenyl, aryl, cycloalkyl, cycloalkylalkyl, heterocyclyl, and heterocyclylalkyl may be substituted or unsubstituted;
   n is 0, 1, or 2,
or a pharmaceutically-acceptable salt thereof.

In some embodiments, the present disclosure provides a compound of formula I-j, wherein R′ and R″ are each independently H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-j, wherein R₁₇ is —NR¹⁵R¹⁶ or —OR₁₅. In some embodiments. R¹⁷ is —OH, —OMe, —NHEt, —NHPh, —NH₂, or —NHCH₂pyridyl.

In some embodiments, the present disclosure provides a compound of formula I-k or I-k-1:

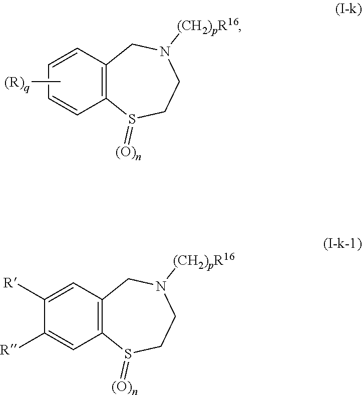

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —Na, —SO₃H, —S(═O)₂alkyl, —S(═O)alkyl, or —OS(═O)CF₃;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
   q is 0, 1, 2, 3, or 4;
   p is 1, 2, 3, 4, 5, 6, 7, 8 9, or 10; and
   n is 0, 1, or 2,
or a pharmaceutically-acceptable salt thereof.

In some embodiments, the present disclosure provides a compound of formula I-k, wherein each R is independently H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R is —OMe at position 7 of the benzothiazepine ring.

In some embodiments, the present disclosure provides a compound of formula I-k-1, wherein R′ and R″ are each independently H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-k or I-k-1, wherein R¹⁸ is —NR¹⁵R¹⁶, —(C—O)OR¹⁵, —OR¹⁵, alkyl that is substituted or unsubstituted, or aryl that is substituted or unsubstituted. In some embodiments, m is 1, and R¹⁸ is -Ph, —C(═O)OMe, —C(═O)OH, aminoalkyl, —NH₂, —NHOH, or NHCbz. In other embodiments, m is 0, and R¹⁸ is C₁-C₄ alkyl. In other embodiments, R¹⁸ is Me, Et, propyl, and butyl. In some embodiments, m is 2, and R¹⁸ is pyrrolidine, piperidine, piperazine, or morpholine. In some embodiments, m is 3, 4, 5, 5, 7, or 8, and R¹⁸ is a fluorescent labeling group selected from bodipy, dansyl, fluorescein, rhodamine, Texas red, cyanine dyes, pyrene, coumarins, Cascade Blue™, Pacific Blue, Marina Blue, Oregon Green, 4′,6-Diamidino-2-phenylindole (DAPI), indopyra dyes, lucifer yellow, propidium iodide, porphyrins, arginine, and variants and derivatives thereof.

In some embodiments, the present disclosure provides a compound of formula of I-l or I-l-1:

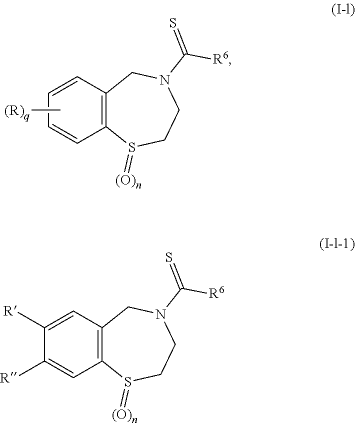

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)CF₃;
   R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)alkyl, —S(═O)alkyl, or —OS(═O)₂CF₃;
   R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   q is 0, 1, 2, 3, or 4; and
   n is 0, 1, or 2,
or a pharmaceutically-acceptable salt thereof.

In some embodiments, the present disclosure provides a compound of formula I-1, wherein each R is independently halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R is —OMe at position 7 of the benzothiazepine ring.

In some embodiments, the present disclosure provides a compound of formula I-1-1, wherein R′ and R″ are each independently H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-1 or I-1-1, wherein R⁶ is acyl, alkenyl, alkyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —OR¹⁵, —NHOH, or —CH₂X. In some embodiments, R⁶ is —NR¹⁵R¹⁶. In some embodiments, R⁶ is —NHPh, pyrrolidine, piperidine, piperazine, morpholine. In some embodiments, R⁶ is alkoxyl. In some embodiments, R⁶ is —O-tBu.

In some embodiments, the present disclosure provides a compound of formula I-m or I-m-1:

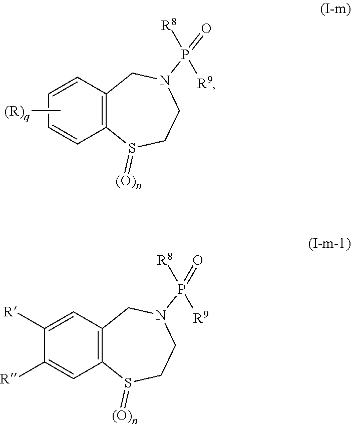

wherein
   n is 0, 1, or 2;
   q is 0, 1, 2, 3, or 4;
   R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃; and
   R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or —OH,
or a pharmaceutically-acceptable salt thereof.

In some embodiments, the present disclosure provides a compound of formula I-m, wherein each R is independently halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments. R is —OMe at position 7 of the benzothiazepine ring.

In some embodiments, the present disclosure provides a compound of formula I-m-1, wherein R′ and R″ are each independently H, halogen, —OH, —OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, -Ph, —NHCH₂Ph, —C(═O)Me, —OC(═O)Me, morpholinyl, or propenyl; and n is 0, 1 or 2. In some embodiments, R′ is H or —OMe, and R″ is H.

In some embodiments, the present disclosure provides a compound of formula I-m or I-m-1, wherein R⁸ and R⁹ are each independently alkyl, aryl, —OH, alkoxyl, or alkylamino. In some embodiments, R⁸ is C₁-C₄alkyl. In some embodiments, R⁸ is Me, Et, propyl or butyl. In some embodiments, R⁹ is aryl. In some embodiments, R⁹ is phenyl.

In some embodiments, the present disclosure provides a compound of formula I-n,

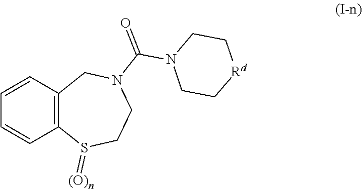

wherein:
   R^{d} is CH₂, or NR^{a}; and
   R^{a} is H, alkoxy, —(C₁-C₆ alkyl)-aryl, wherein the aryl is a disubstituted phenyl or a benzo[1,3]dioxo-5-yl group, or a Boc group.
or a pharmaceutically-acceptable salt thereof.

In some embodiments, R^{a} is H.

Representative compounds of Formula I-n include without limitation S101, S102, S103, and S114.

In some embodiments, the present disclosure provides a compound of Formula I-o:

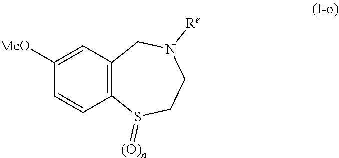

wherein:
   R^{e} is —(C₁-C₆ alkyl)-phenyl, —(C₁-C₆ alkyl)-C(O)R^{b}, or substituted or unsubstituted —C₁-C₆ alkyl; and
   R^{b} is —OH or —O—(C₁-C₆ alkyl),
wherein the phenyl or the substituted alkyl is substituted with one or more of halogen, hydroxyl, —C₁-C₆ alkyl, —O—(C₁-C₆ alkyl), —NH₂, —NH(C₁-C₆ alkyl), —N(C₁-C₆ alkyl)₂, cyano, or dioxolane,
or a pharmaceutically-acceptable salt thereof.

Representative compounds of Formula I-o include without limitation S107, S110, S111, S120, and S121.

In some embodiments, the present disclosure provides a compound of Formula I-p:

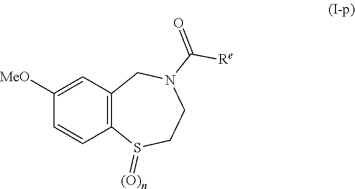

wherein:
   R^{e} is —(C₁-C₆ alkyl)-NH₂, —(C₁-C₆ alkyl)-OR^{f}, wherein R_{f} is H or —C(O)—(C₁-C₆)alkyl, or —(C₁-C₆ alkyl)-NHR^{g}, wherein R^{g} is carboxybenzyl.

In some embodiments, the present disclosure provides compounds of Formula II or Formula III:

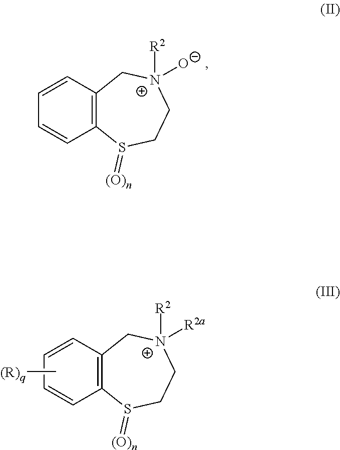

wherein:
   n is 0, 1, or 2;
   q is 0, 1, 2, 3, or 4;
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)CF₃;
   each R² and R^{2a} is independently alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or —OH;
   each R¹⁰ is —NR¹⁵R¹⁶, —OH, —SO₂R¹¹, —NHSO₂R¹¹, —C(═O)R¹², —NH(C═O)R¹², —O(C—O)R¹², or —P(═O)R¹³R¹⁴; m is 0, 1, 2, 3, or 4;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, —OH, —NH₂, —NHNH₂, or —NHOH;
   each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
   each R¹⁵ and R¹⁶ is independently acyl, alkenyl, alkoxyl, —OH, —NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹³ and R¹⁸ together with the N to which R¹⁵ and R¹⁶ are bonded form a heterocycle that is substituted or unsubstituted;

or a pharmaceutically-acceptable salt thereof.

In some embodiments, the compound of formula (I) is selected from:

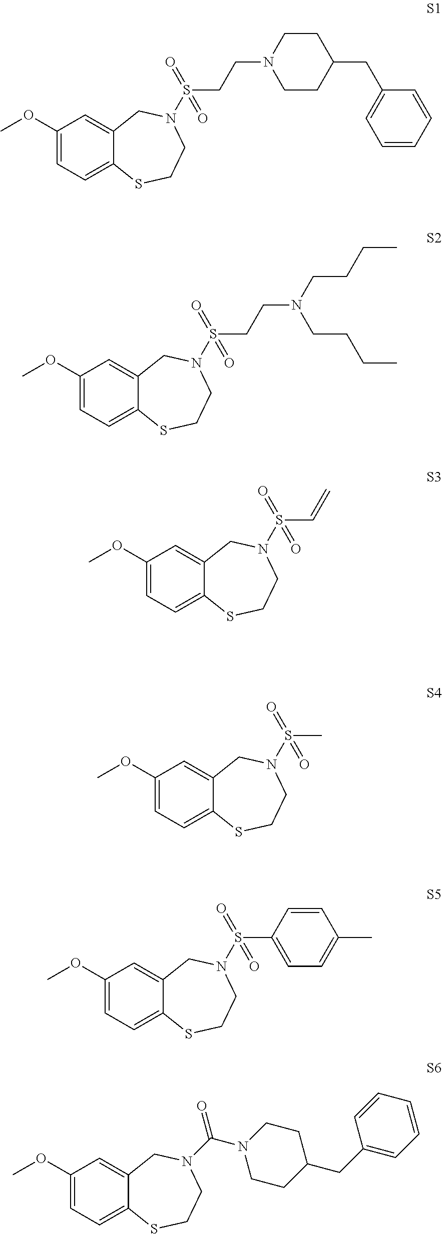

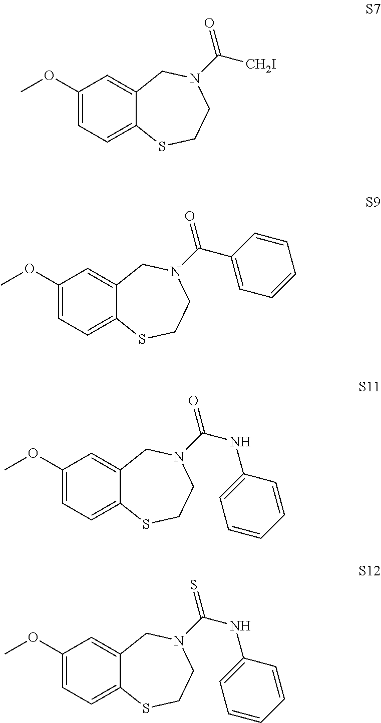

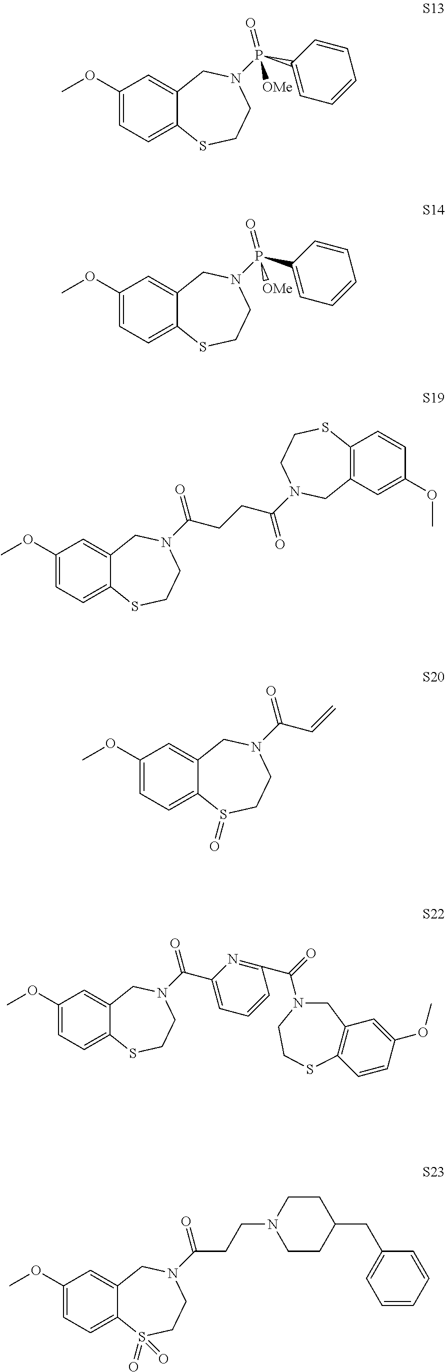

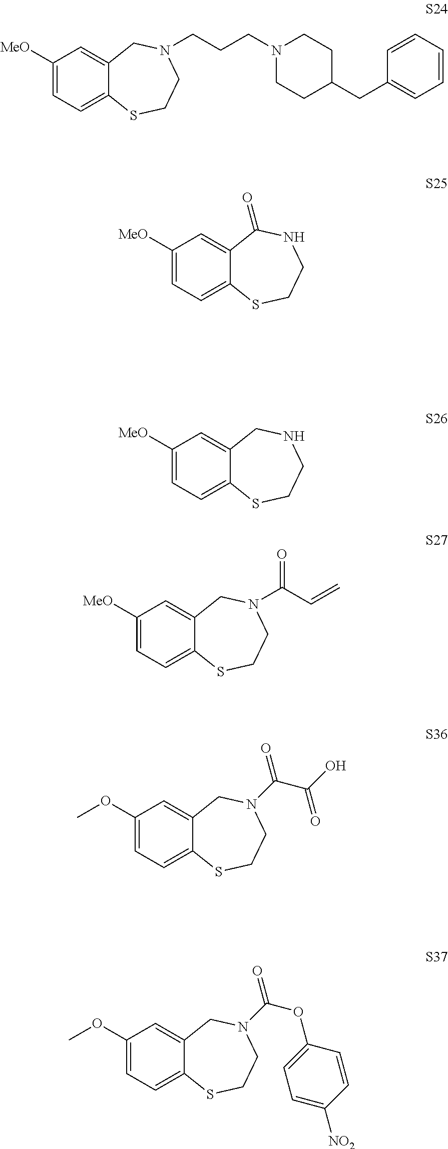 [SMILES: COc1ccc2c(c1)CN(CCCN1CCC(Cc3ccccc3)CC1)CCS2 ; COc1ccc2c(c1)C(=O)NCCS2 ; COc1ccc2c(c1)CNCCS2 ; C=CC(=O)N1CCSc2ccc(OC)cc2C1 ; COc1ccc2c(c1)CN(C(=O)C(=O)O)CCS2 ; COc1ccc2c(c1)CN(C(=O)Oc1ccc([N+](=O)[O-])cc1)CCS2]

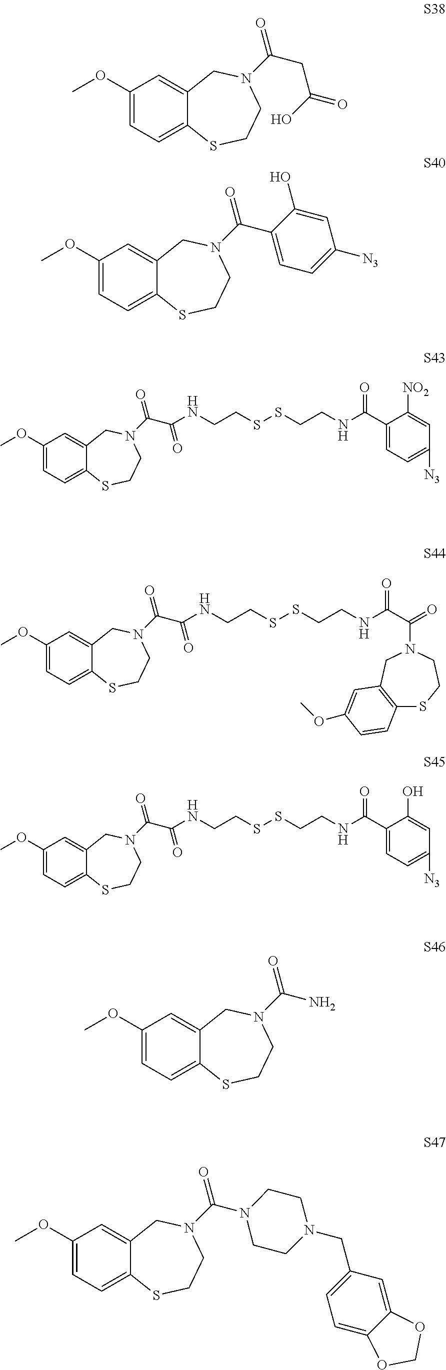

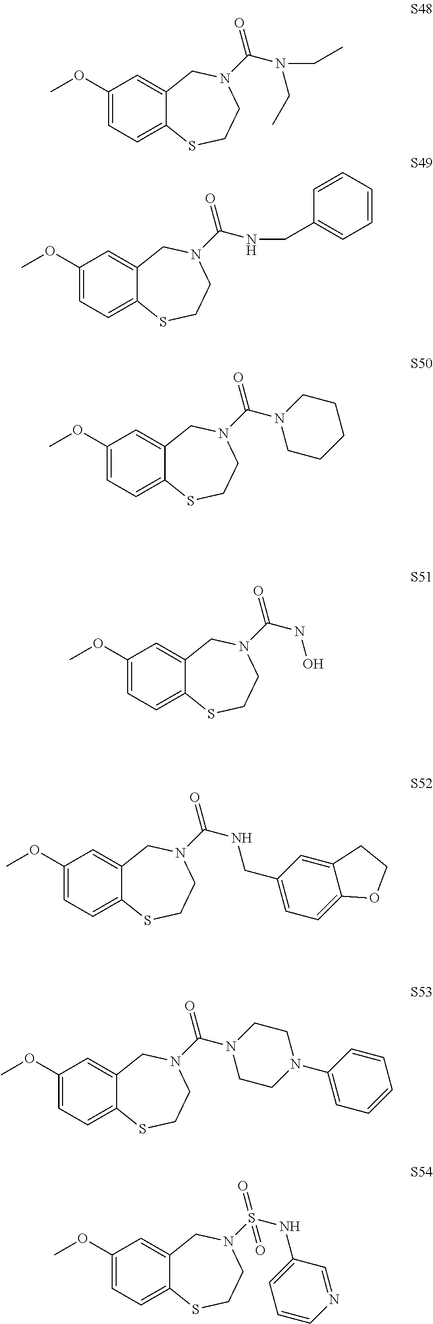

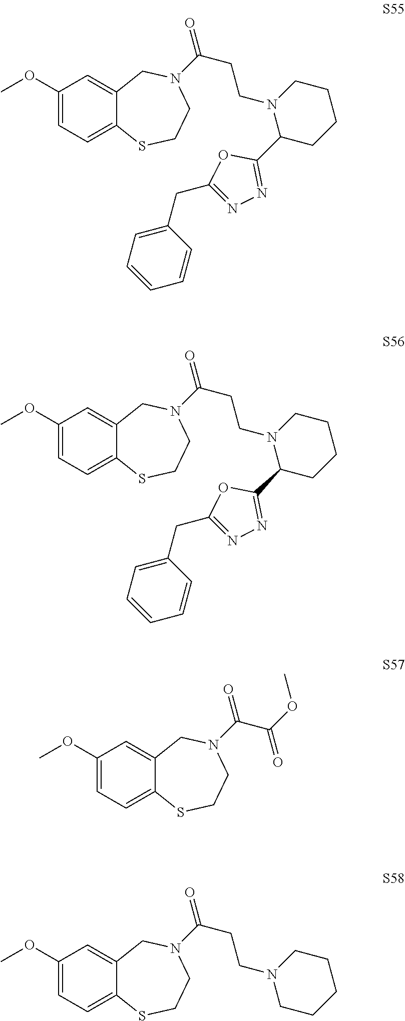 [SMILES: COc1ccc2c(c1)CN(C(=O)CCN1CCCCC1c1nnc(Cc3ccccc3)o1)CCS2 ; COc1ccc2c(c1)CN(C(=O)CCN1CCCC[C@H]1c1nnc(Cc3ccccc3)o1)CCS2 ; COC(=O)C(=O)N1CCSc2ccc(OC)cc2C1 ; COc1ccc2c(c1)CN(C(=O)CCN1CCCCC1)CCS2]

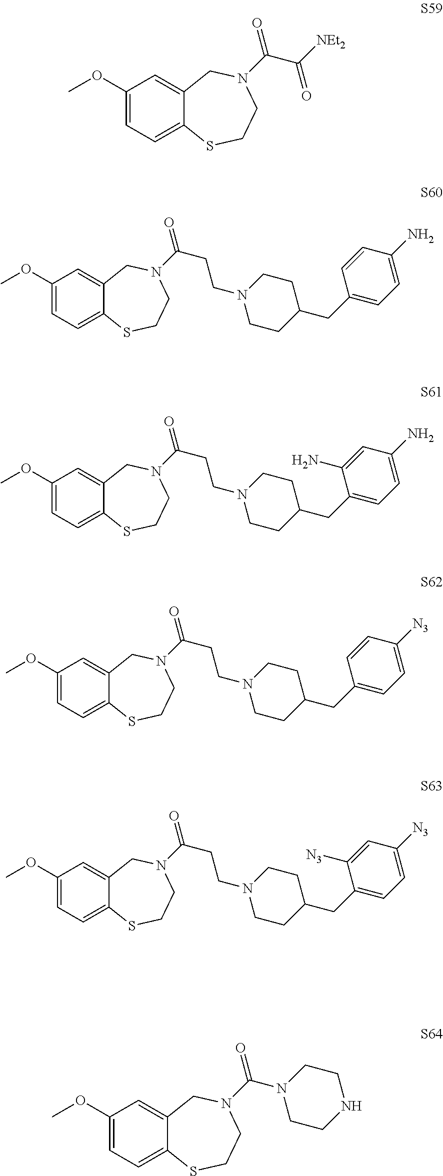

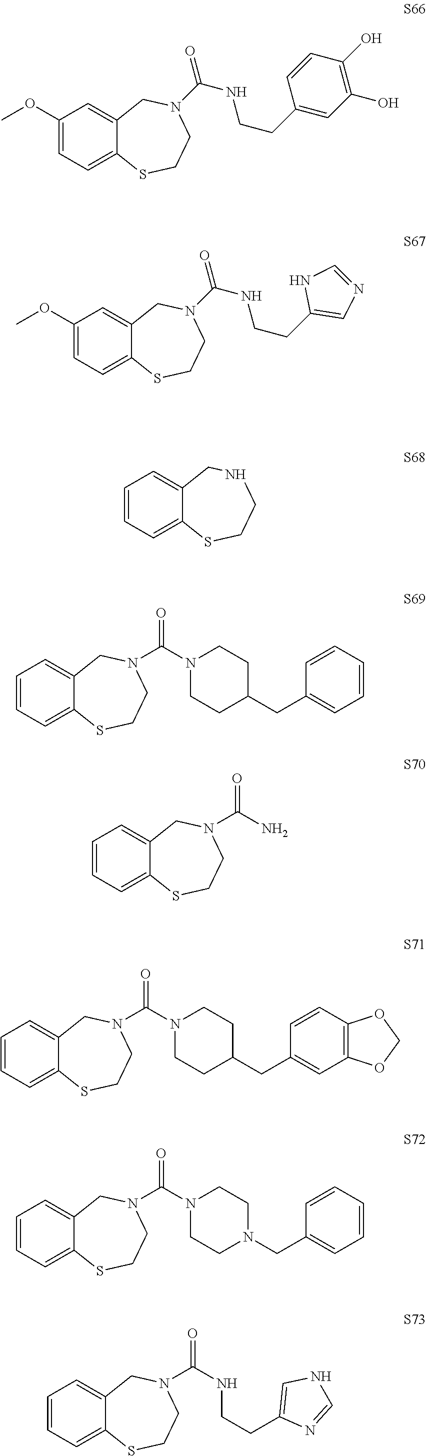

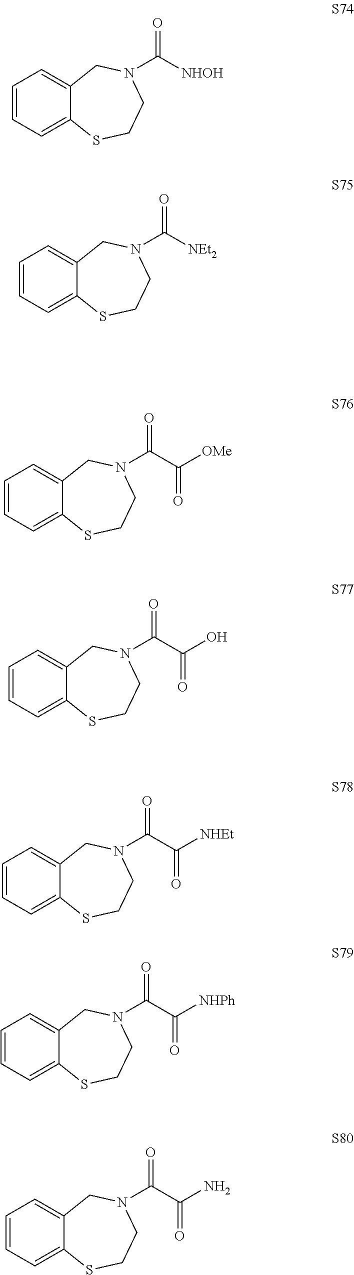

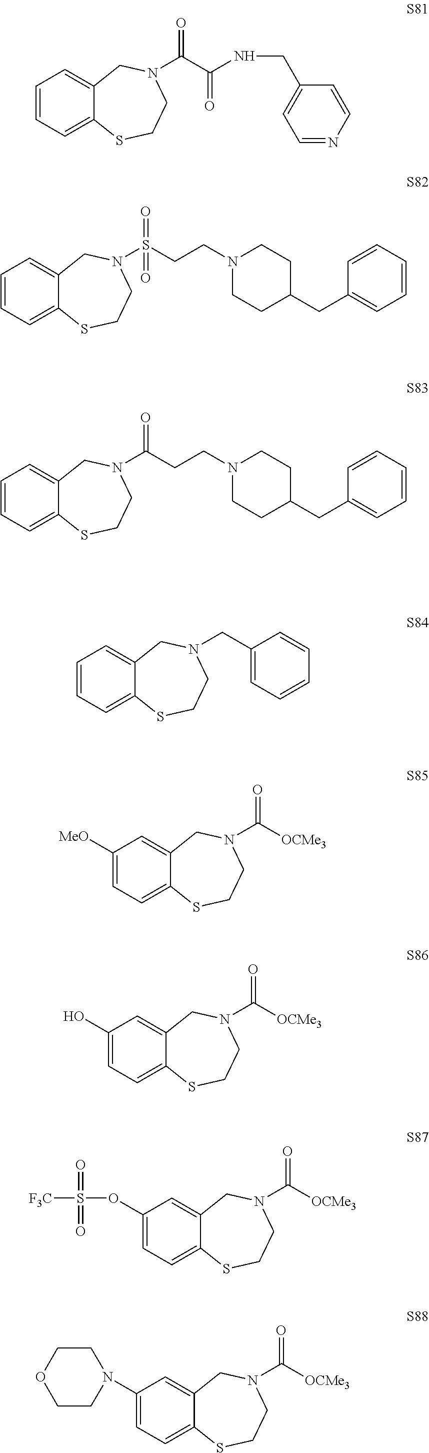

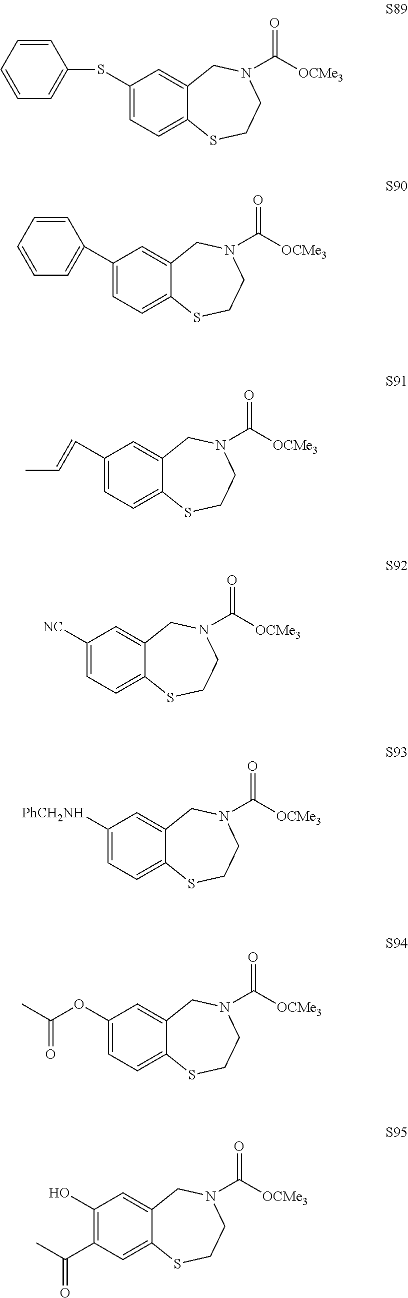

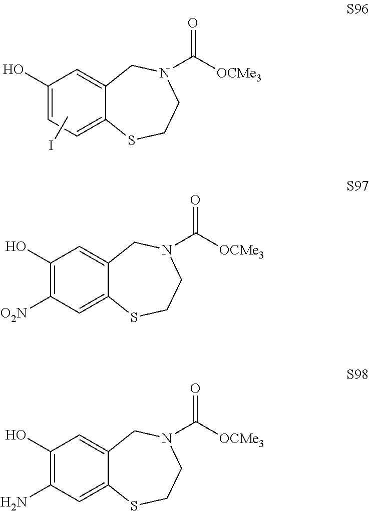

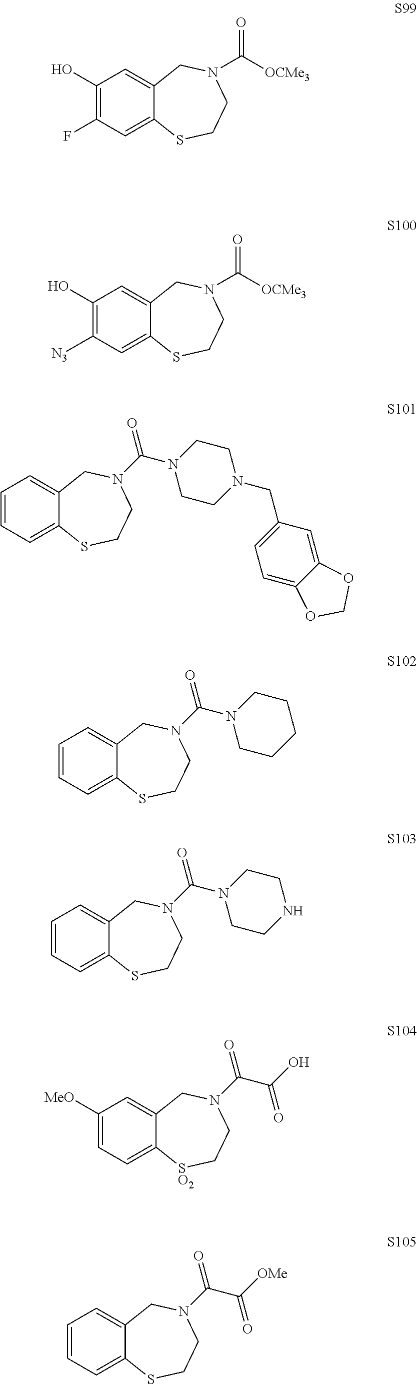

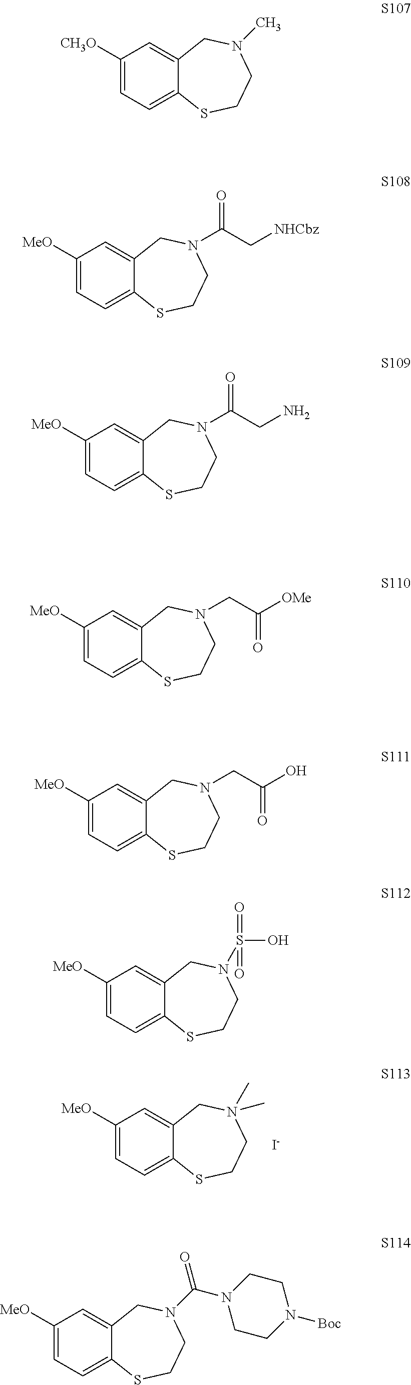

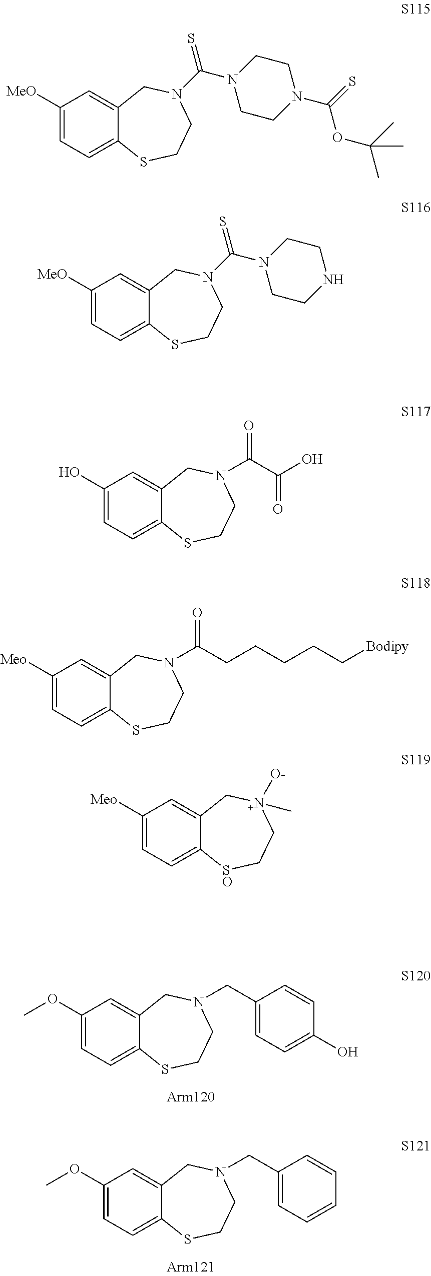

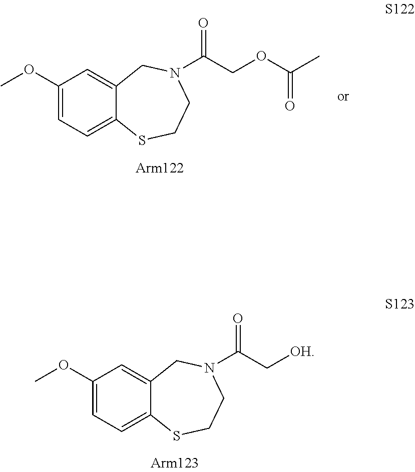

In some embodiments, the compound is a compound of Formula (I), (I-a), (I-b), (I-c), (I-d), (I-e), (I-f), (I-g), (I-h), (I-i), (I-j), (I-k), (I-k-1), (I-1), (I-1-1), (1-m), (I-m-1), (I-n), (1-0), (I-p), (II), or (III). In some embodiments, the compound is S1, S2, S3, S4, S5, S6, S7, S9, S11, S12, S13, S14, S19, S20, S22, S23, S24, S25, S26, S27, S36, S37, S38, S40, S43, S44, S45, S46, S47, S48, S49, S50, S51, S52, S53, S54, S55, S56, S57, S58, S59, S60, S61, S62, S63, S64, S66, S67, S68, S69, S70, S71, S72, S73, S74, S75, S76, S77, S78, S79, S80, S81, S82, S83, S84, S85, S86, S87, S88, S89, S90, S91, S92, S93, S94, S95, S96, S97, S98, S99, S100, S101, S102, S103, S104, S105, S107, S108, S109, S110, S111, S112, S113, S114, S115, S116, S117, S118, S119, S120, S121, S122, or S123, as herein defined.

In an embodiment, the preferred calcium channel stabilizer is one that can penetrate the blood brain barrier to directedly administer treatment to the brain.

In an embodiment, the calcium channel stabilizer is represented by the structure

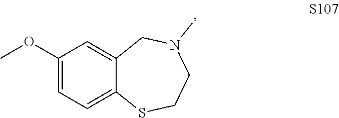

or a salt thereof, such as the HCl salt.

In an embodiment, the calcium channel stabilizer is represented by the structure

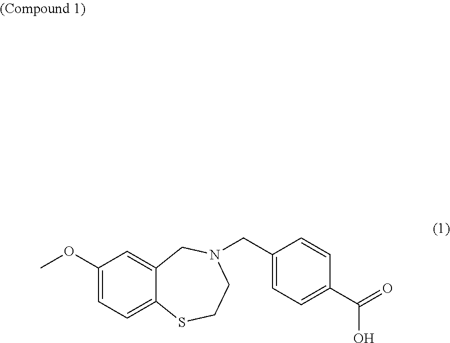 [SMILES: COc1ccc2c(c1)CN(Cc1ccc(C(=O)O)cc1)CCS2]

or a pharmaceutically-acceptable salt thereof.

### Pharmaceutical Compositions

Compounds can be formulated into pharmaceutical compositions for administration to human subjects in a biologically compatible form suitable for administration in vivo. According to another aspect, compounds are formulated into pharmaceutical compositions in admixture with a pharmaceutically-acceptable diluent and/or carrier. The pharmaceutically-acceptable carrier must be “acceptable” in the sense of being compatible with the other ingredients of the composition and not deleterious to the recipient thereof. The pharmaceutically-acceptable carrier employed herein is selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. If necessary, pharmaceutical additives, such as antioxidants, aromatics, colorants, flavor-improving agents, preservatives, and sweeteners, are also added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others.

The pharmaceutical formulations can be brought into association with a carrier and/or diluent, as a suspension or solution. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

In some embodiments, compounds can be administered to a human or animal subject by known procedures including, without limitation, oral administration, sublingual or buccal administration, parenteral administration, transdermal administration, via inhalation or intranasally, vaginally, rectally, and intramuscularly. The compounds of the disclosure can be administered parenterally including, without limitation, by epifascial, intracapsular, intracranial, intracutaneous, intrathecal, intramuscular, intraorbital, intraperitoneal, intraspinal, intrasternal, intravascular, intravenous, parenchymatous, subcutaneous or sublingual injection, or by way of catheter. In one embodiment, the agent is administered to the subject by way of delivery to the subject's muscles including, but not limited to, the subject's cardiac muscles. In an embodiment, the agent is administered to the subject by way of targeted delivery to cardiac muscle cells via a catheter inserted into the subject's heart.

For oral administration, a formulation of the compounds described herein may be presented, for example and without limitation, as capsules, tablets, powders, granules, or as a suspension or solution. The formulation can include conventional additives, such as lactose, mannitol, cornstarch or potato starch. The formulation also can be presented with binders, such as crystalline cellulose, cellulose derivatives, acacia, cornstarch or gelatins. Additionally, the formulation can be presented with disintegrators, such as cornstarch, potato starch or sodium carboxymethylcellulose. The formulation also can be presented with dibasic calcium phosphate anhydrous or sodium starch glycolate. Finally, the formulation can be presented with lubricants, such as talc or magnesium stearate.

For parenteral administration (i.e., administration by injection through a route other than the alimentary canal), the compounds can be combined with a sterile aqueous solution that is isotonic with the blood of the subject. Such a formulation is prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulation is presented in unit or multi-dose containers, such as sealed ampoules or vials. The formulation is delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual or by way of catheter into the subject's heart.

For transdermal administration, the compounds can be combined with skin penetration enhancers, such as propylene glycol, polyethylene glycol, isopropanol, ethanol, oleic acid, N-methylpyrrolidone and the like, which increase the permeability of the skin to the compounds of the disclosure and permit the compounds to penetrate through the skin and into the bloodstream. The compound/enhancer compositions also may be further combined with a polymeric substance, such as ethylcellulose, hydroxypropyl cellulose, ethylene/vinylacetate, polyvinyl pyrrolidone, and the like, to provide the composition in gel form, which are dissolved in a solvent, such as methylene chloride, evaporated to the desired viscosity and then applied to backing material to provide a patch.

In some embodiments, in order to prepare the pharmaceutical composition, the calcium channel stabilizer, as the active ingredient, is intimately admixed with a pharmaceutically-acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be provided in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by inhalation or insufflation. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. Concentrations of the calcium channel stabilizer will typically be in the range of about 1 to about 10 mg/kg and preferably about 2 to about 5 mg/kg based on the weight of the subject. A therapeutically effective amount of the calcium channel stabilizer or an amount effective to treat cognitive dysfunction associated with a respiratory virus infection may be specifically determined initially from the Example described herein and adjusted as necessary using routine methods.

### EXAMPLES

### Example 1: Neuronal RyR2 Channels are Leaky in Individuals with Heart Failure

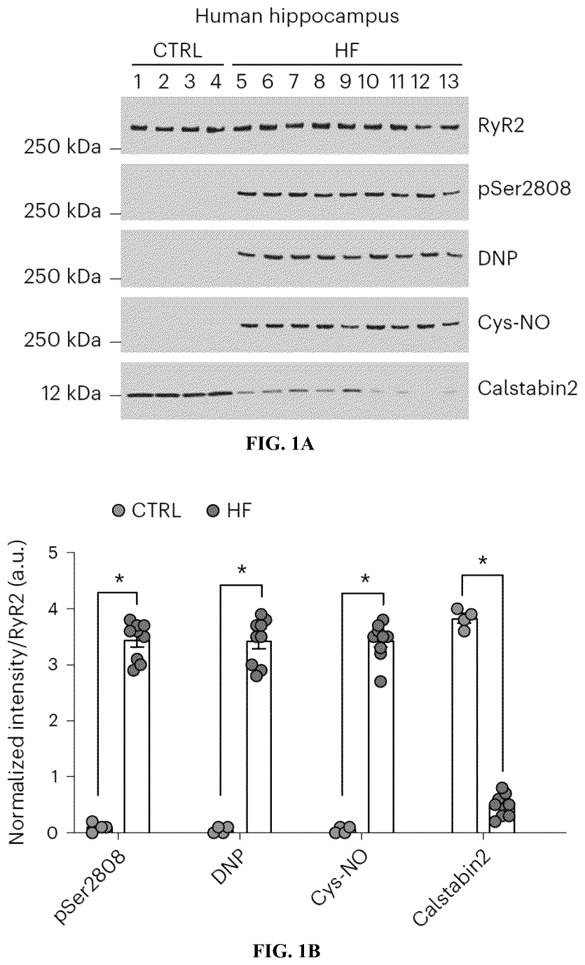

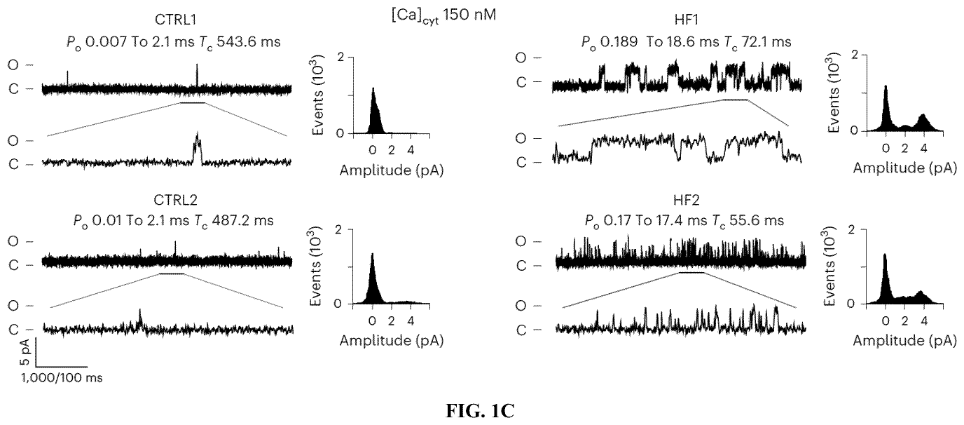

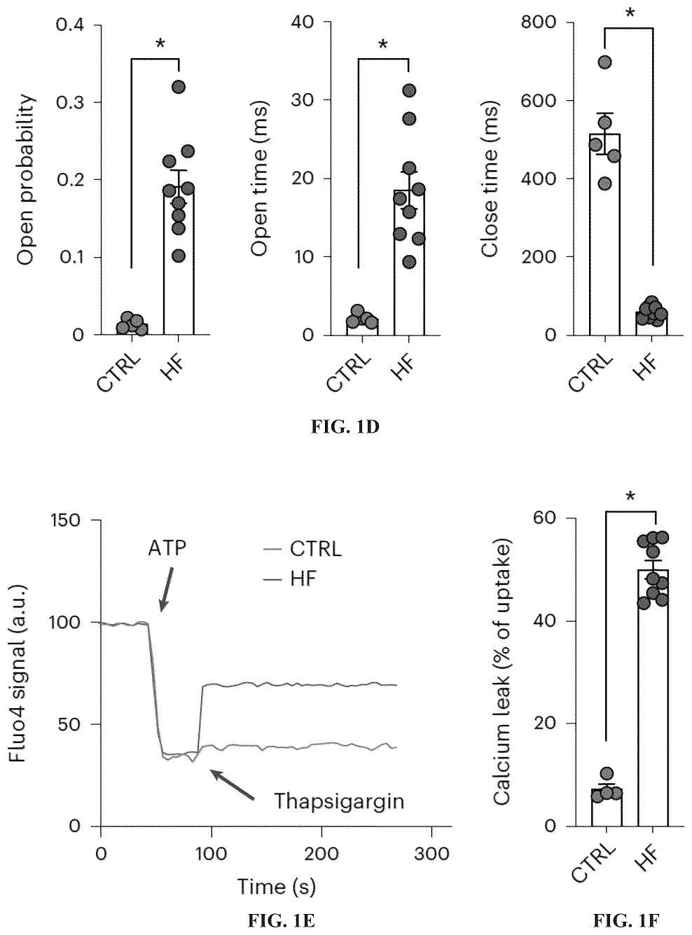

To evaluate RyR2 in the brains of individuals with HF, hippocampal biopsy samples from controls (non-HF) and de-identified individuals with HF were obtained from the Brain Bank at Columbia University and the National Institutes of Health (NIH) Neuro-Biobank. Immunoprecipitated RyR2 and isolated ER fractions were used to analyze the composition of the hippocampal RyR2 macromolecular complex and PTMs known to be associated with RyR channel Ca²⁺ leak. Hippocampal RyR2 from individuals with HF (n=9) exhibited PKA hyper-phosphorylation (on Ser2808), oxidation, cysteine nitrosylation, and were depleted of calstabin2, compared to controls (n=4) as shown in FIGS. 1A and 1B. This is the ‘biochemical signature’ of ‘leaky’ RyR2 channels. Single-channel recordings of hippocampal RyR2, reconstituted into planar lipid bilayers, revealed increased open probability (Po), increased mean open time (To) and decreased mean closed time (Tc) in individuals with HF compared to controls (Po=0.19%±0.02%, To=18±2 ms, Tc=58±05 ms in HF hippocampi, n=9; versus Po=0.01%±0.002%, To=2±0.2 ms, Tc=515±52 ms in controls, n=4; P<0.05) in the presence of low, non-activating [Ca²⁺]cis (150 nM), conditions under which normal RyR2 channels are tightly closed, as shown in FIGS. 1C and 1D. This elevated Po is consistent with pathological hippocampal ER Ca²⁺ leak. Indeed, neuronal microsomes from individuals with HF exhibited increased RyR-mediated ER Ca²⁺ leak compared with controls as shown in FIGS. 1E and 1F.

### Example 2: Impaired Cognitive Function in Mouse Model of Heart Failure

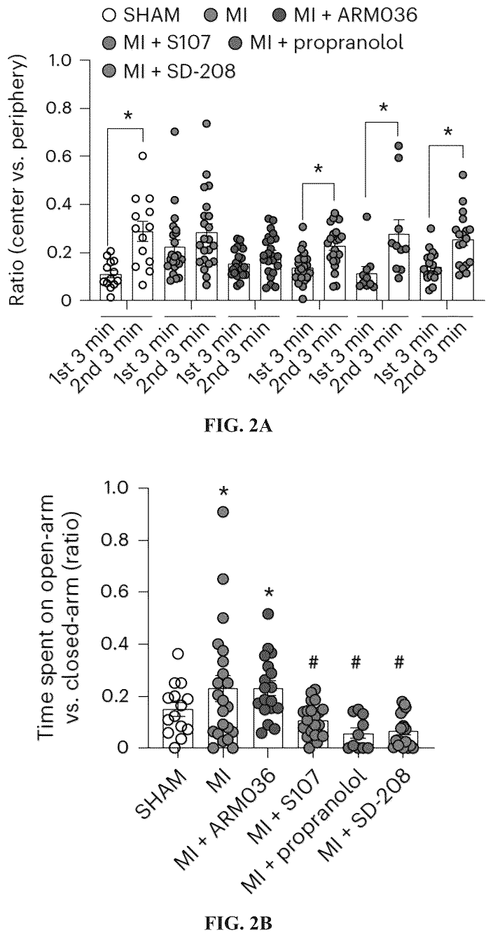

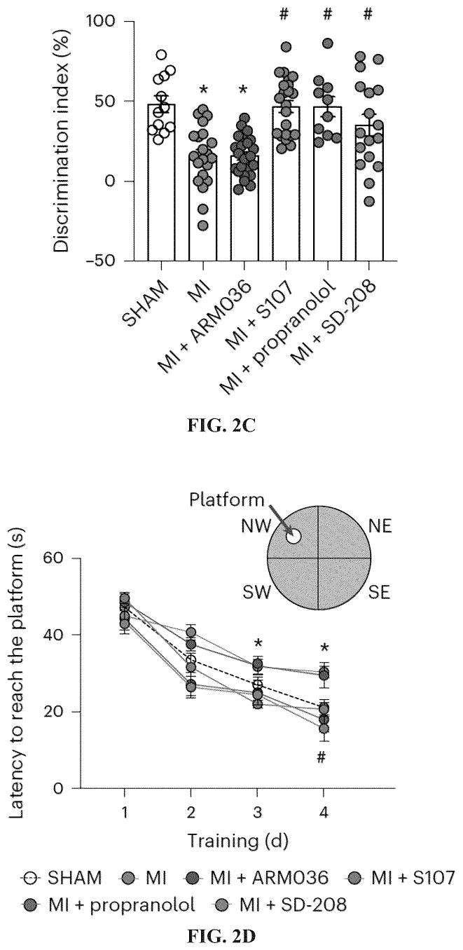

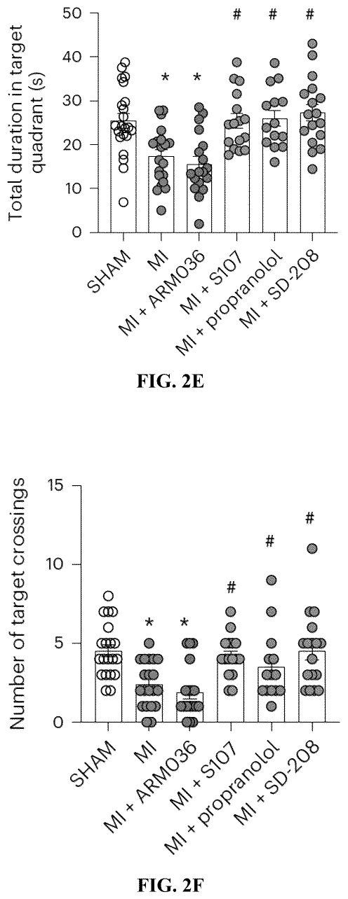

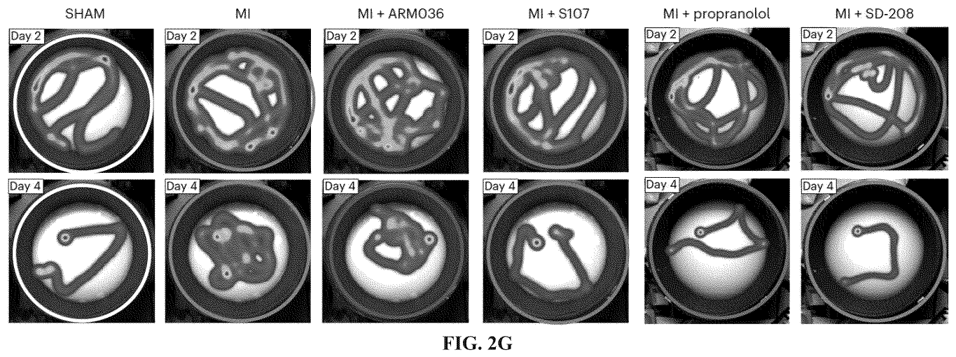

Because of the complexity of the clinical manifestation of HF patients, the mouse model of HF (myocardial infarction, MI) was used with reduced ejection fraction to evaluate the mechanisms of CD. The open field test and elevated plus maze (EPM) test were used to evaluate the behavioral phenotypes, and spontaneous exploratory activity in the mice. In the open field test, mice were placed at the center of a chamber and allowed to explore for 6 min. Within the first and the second 3 min, the ratio of time spent in the center versus periphery area for MI mice was similar (0.22±0.02 and 0.28±0.03, n=22, P=0.18), whereas these ratios were significantly different in the SHAM group (0.1±0.01 and 0.28±0.04, n=13, P<0.05; FIG. 2A). In the EPM test, MI mice spent more time in the open arms of the EPM (FIG. 2B) compared to SHAM (0.22±0.04 versus 0.14±0.02; P<0.05). The abnormal behaviors were prevented by the pharmacological treatments using the RyR2 stabilizer Rycal® compound S107, the nonselective beta-adrenergic antagonist propranolol, and the anti-inflammatory transforming growth factor-beta (TGF-β) inhibitor SD-208 (FIGS. 2A and 2B). As a control, the effects of a Rycal®, ARM036, was studied. The structure of ARM036 is:

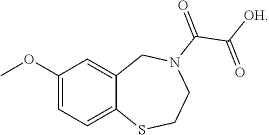 [SMILES: COc1ccc2c(c1)CN(C(=O)C(=O)O)CCS2]

That compound has the same mechanism of action as S107 but does not cross the blood-brain barrier (BBB) while S107 does. ARM036 had no effect on MI mouse cognitive performance in either task (FIGS. 2a and 2b) indicating that the S107 effects were at the CNS level rather than the systemic level.

Next, a novel object recognition test was used to evaluate hippocampal-dependent short-term memory. (see Liu, X. et al. Role of leaky neuronal ryanodine receptors in stress-induced cognitive dysfunction. Cell 150, 1055-1067 (2012); and Bevins, R. A. & Besheer, J. Object recognition in rats and mice: a one-trial non-matching-to-sample learning task to study ‘recognition memory’. Nat. Protoc. 1, 1306-1311 (2006)). MI mice showed significantly lower discrimination index (15%±4%) compared to the SHAM group (48%±5%, P<0.05; FIG. 2C). S107, propranolol and SD-208 treatments prevented short-term memory loss by increasing the discrimination index to 46%±4%, 46%±6% and 35%±6%, respectively (P<0.05), whereas ARM036 did not increase the discrimination index (15%±3%; P=0.25; FIG. 2C).

A MWM test was performed to assess hippocampal-dependent long-term spatial learning and memory. MI mice exhibited significantly prolonged latency to find and reach the hidden platform on day 4 training trials (30±1 s, n=20) compared to the SHAM controls (21±2 s, P<0.05, n=22). S107, propranolol and SD-208 treatments significantly reduced latency on day 4 (20±2 s, 17±3 s and 15±3 s, respectively, P<0.05, n=19, 14 and 19 per group). Again, MI mice treated with ARM036 did not show significant reduction on the latency to reach the hidden platform (29±3 s, P=0.99, n=19; FIG. 2D).

A probe trial was performed on day 5 of the MWM test. MI mice spent a significantly shorter duration in target quadrant (17±1 s, n=19) and exhibited a reduced number of target crossings (2.4±0.3, n=19) within the 60-s probe trial compared to SHAM (25±2 s and 4.6±0.3, P<0.05, n=20; FIGS. 2E and 2F). S107, propranolol and SD-208 treatments, but not ARM036, were able to correct the spatial memory deficit in MI by improving the time spent in the target quadrant (26±2 s, 26±2 s and 27±2 s, respectively, P<0.05) and target crossings (4.2±0.3, 3.5±0.6 and 4.5±0.6, respectively, P<0.05, n=14-19 per group) in the probe trial (FIGS. 2E and 2F). A heat map of the swimming behavior of these mice is shown at days 2 and 4 (FIG. 2G). Of note, the traveled distance and velocity of mice in the open field, EPM and MWM tests were comparable between all the groups.

### Example 3: Neuronal RyR2 Channels are Leaky in the Mouse Model of Heart Failure

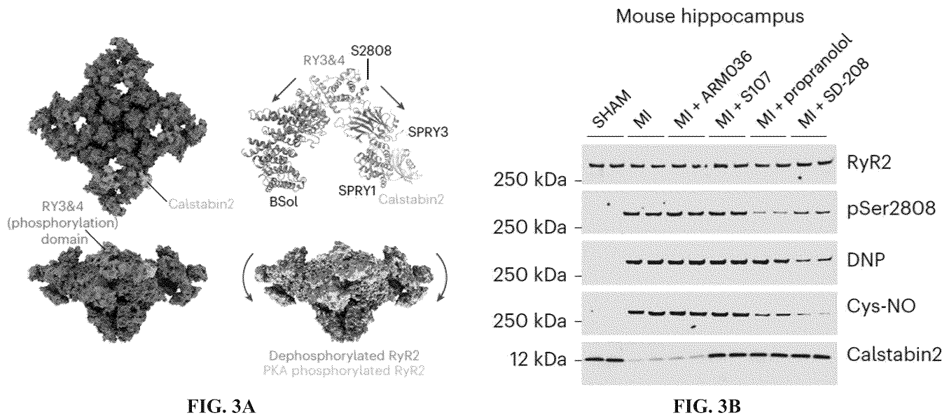

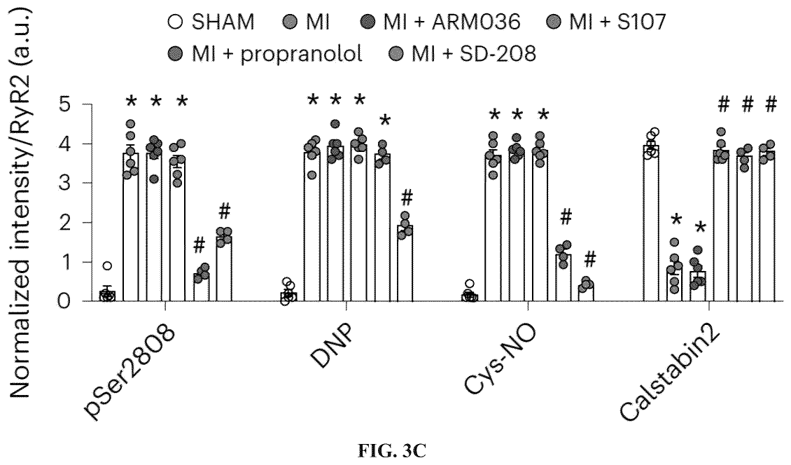

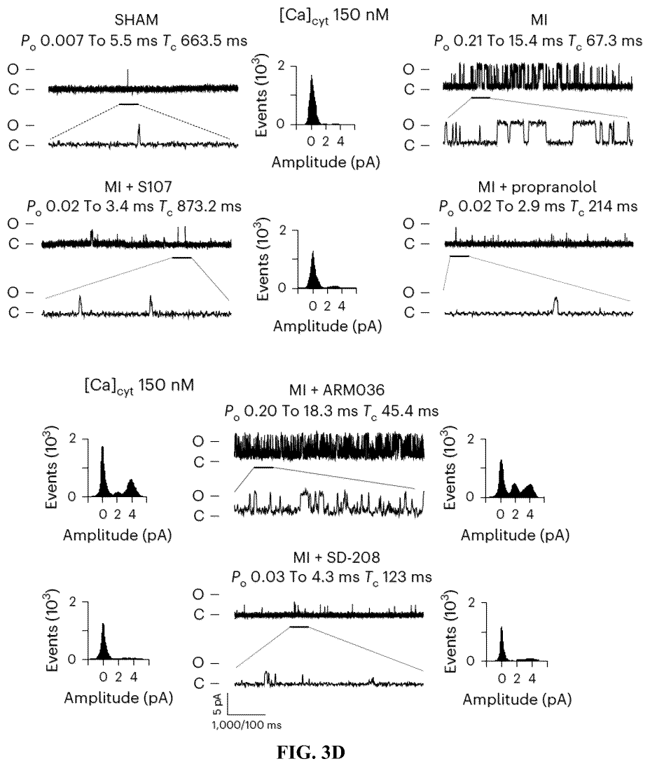

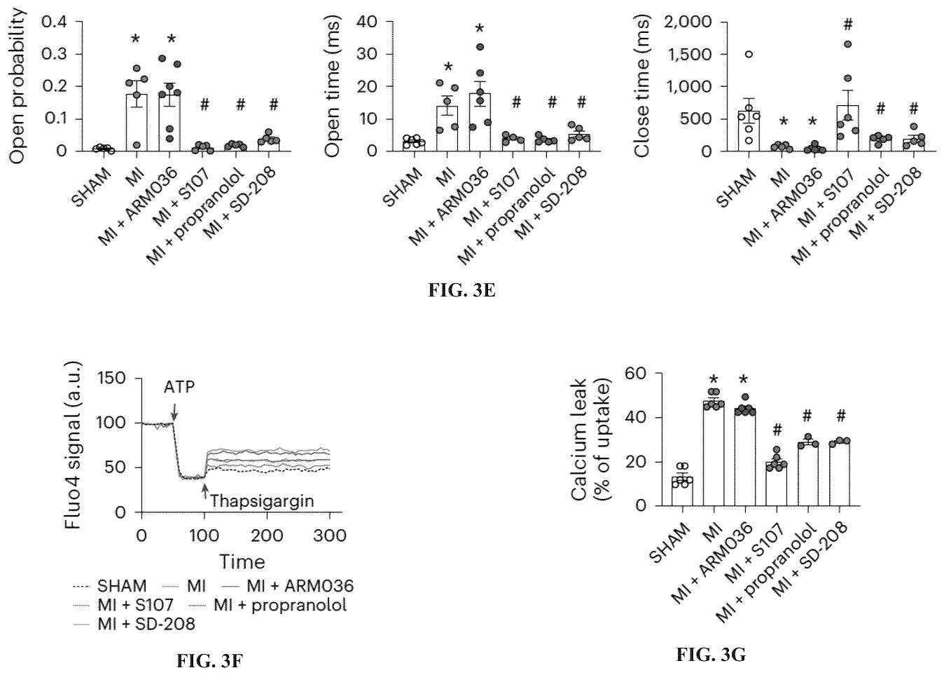

The review of a previously conducted high-resolution three-dimensional (3D) structure of human RyR2 showed that the PKA-phosphorylated channels on Ser2808 adopt a primed state (halfway between the closed and the open states), allowing the opening of channels at a lower cytosolic Ca²⁺ concentration resulting in leaky channels (FIG. 3A). The previously identified binding site of the Rycal® drugs (S107 and Compound 1) on RyR1 and RyR2 shows that these drugs are able to reverse the primed state of leaky channels toward a fully closed state. PTMs and functional abnormalities of neuronal RyR2 in HF hippocampal samples from MI mice were then evaluated and compared them to SHAM. Hippocampal RyR2 was immunoprecipitated and immunoblotted to detect PTMs. Neuronal RyR2 from MI mice exhibited PKA hyper-phosphorylation (on Ser2808, the main PKA-phosphorylation site), oxidation, cysteine nitrosylation and calstabin2 depletion compared to the SHAM hippocampal samples (P<0.05; FIGS. 3B and 3C). Single-channel recordings of neuronal RyR2 from MI mice revealed increased open probability (Pₒ=0.17%±0.04%) in the presence of low non-activating [Ca²⁺]_{cis}, (SHAM, Pₒ=0.07%±0.002%, P<0.05). Hippocampal RyR2 from the MI mice also exhibited increased open time and decreased close time (FIGS. 3D and 3E). This elevated Pₒ is consistent with pathological ER Ca²⁺ leak. Indeed, neuronal microsomes from MI mice exhibited increased RyR-mediated ER Ca²⁺ leak compared with SHAM-operated mice (FIGS. 3F and 3G). Interestingly, PTMs and functional RyR2 remodeling (leak) were comparable to the RyR2 abnormalities observed in human HF hippocampal samples (FIGS. 1A-1F). S107 treatment (but not ARM036 treatment) restored calstabin2 binding to RyR2 and decreased the hippocampal RyR2 open probability (Pₒ=0.01%±0.003%, P<0.05) and mean open time, and increased mean closed time, and reduced ER Ca²⁺ leak (FIGS. 3E through 3G) indicating that the ability of S107 to cross the BBB was essential to fix the leaky hippocampal RyR2 channels. Propranolol treatment significantly reduced RyR2 phosphorylation, nitrosylation and restored calstabin2 binding to the channels. Meanwhile SD-208 treatment significantly decreased RyR2 phosphorylation, oxidation/nitrosylation and enabled rebinding of calstabin2 to the channels (FIGS. 3B and 3C). Both propranolol and SD-208 treatment decreased RyR2 open probability (Pₒ=0.018%±0.020% and 0.030%=0.005%, respectively) and mean open time, and increased mean closed time and reduced the ER Ca²⁺ leak compared to the untreated MI mice (FIGS. 3E through 3G). As mentioned above, S107 is a specific RyR2-targeted drug that stabilizes the closed conformation of the channels without effects on PTMs, whereas propranolol and SD-208 prevent RyR2 Ca²⁺ leak by reducing the channel oxidation, nitrosylation and hyper-phosphorylation. Thus, a reduction of RyR2 PTMs and/or stabilization of specific interacting domains of the channels allow calstabin2 to rebind and rescue aberrant RyR2 opening and pathological ER Ca²⁺ leak.

### Example 4: Impaired Long-Term Potentiation and Hippocampal Glucose Metabolism in Myocardial Infarction Mice

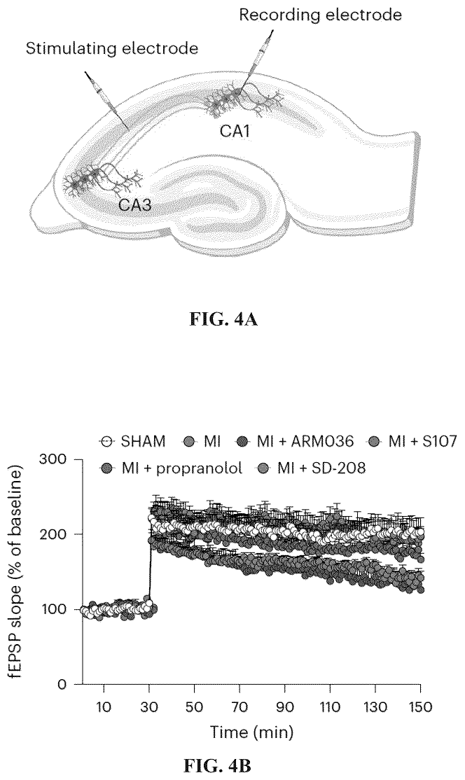

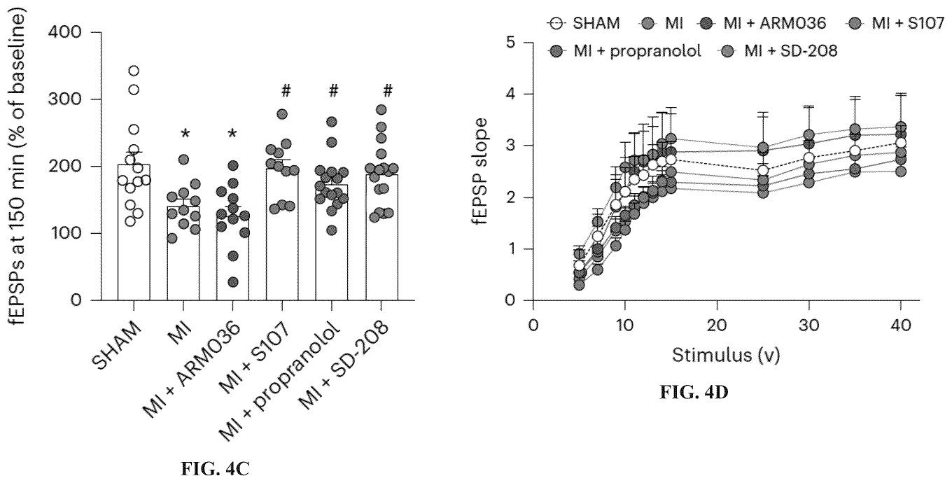

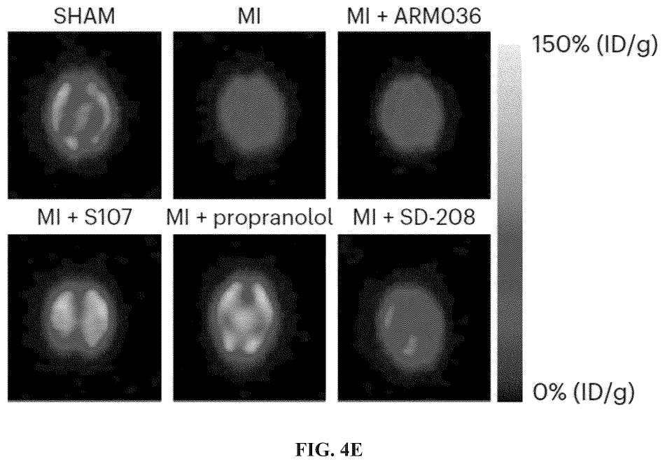

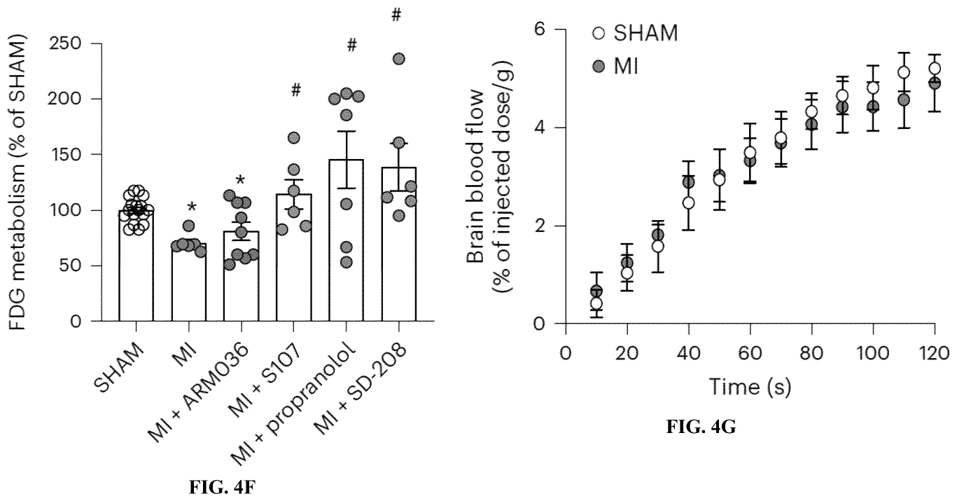

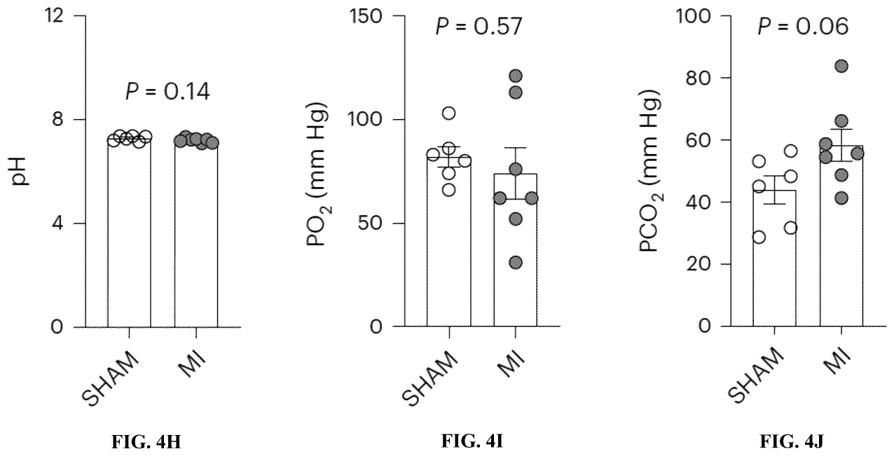

Field excitatory postsynaptic potentials (fEPSPs) were evaluated at the Schaffer collateral using a bipolar electrode placed at the CA3 and recording at the CA1 (FIG. 4A). Hippocampal slices obtained from MI mice showed decreased LTP as compared to slices obtained from SHAM animals (P=0.01). S107 treatment reversed this effect (P=0.001), with slices eliciting the same LTP response as SHAM, while treatment with ARM036 had no effect on hippocampal synaptic plasticity, resulting in impaired LTP in comparison to SHAM animals (P=0.01). In addition, treatments with propranolol (P=0.01) and SD-208 (P=0.005) prevented MI-induced LTP deficits (FIGS. 4B and 4C). Importantly, MI and treatment with the various compounds after MI had no effect on basal synaptic transmission determined by the input-output curves (I-O; P=0.5; FIG. 4D).

Thereafter, [¹⁸F]fluorodeoxyglucose (FDG) with positron emission tomography (PET) was used to measure energy consumption in neurons, which reflects neuronal communication signals and the integrative local neuronal activity. Deteriorating brain glucose metabolism measured by FDG-PET has been used as a clinical marker for Alzheimer's disease diagnosis. Thus, hippocampal FDG metabolism was evaluated in the experimental groups (FIGS. 4E and 4F). Interestingly, hippocampal FDG metabolism was significantly reduced by ˜30% in the MI-treated (70±3, P<0.05) and MI+ARM036-treated (80±8, P<0.05) groups compared to SHAM (100±3). The BBB-permeant drug S107, propranolol and SD-208 significantly improved the hippocampal FDG metabolism. (114±13, 145±26 and 138±22, respectively, P<0.05; FIGS. 4E and 4F). Importantly, the reduced FDG metabolism in the hippocampi of MI mice was not the result of changes in brain blood flow, which were not reduced in HF mice as measured by a dynamic microPET scan of MI and SHAM mice, nor were there any significant changes found in the blood gases including pH, O₂ or CO₂ levels, at 2 months after MI (FIGS. 4G-4J). Both decreased LTP and FDG metabolism are likely due to impaired neurotransmission.

### Example 5: RyR2-p.Ser2808Ala Mice are Protected Against Cognitive Dysfunction

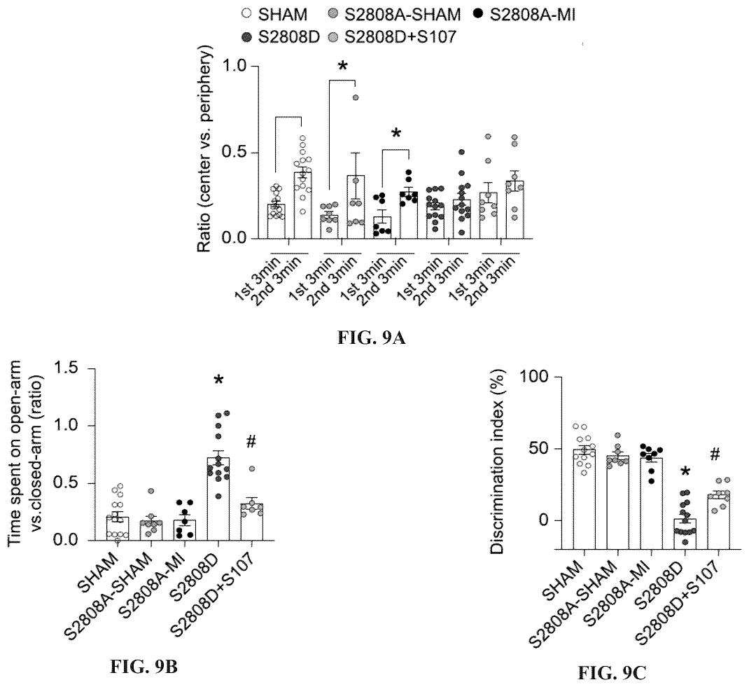

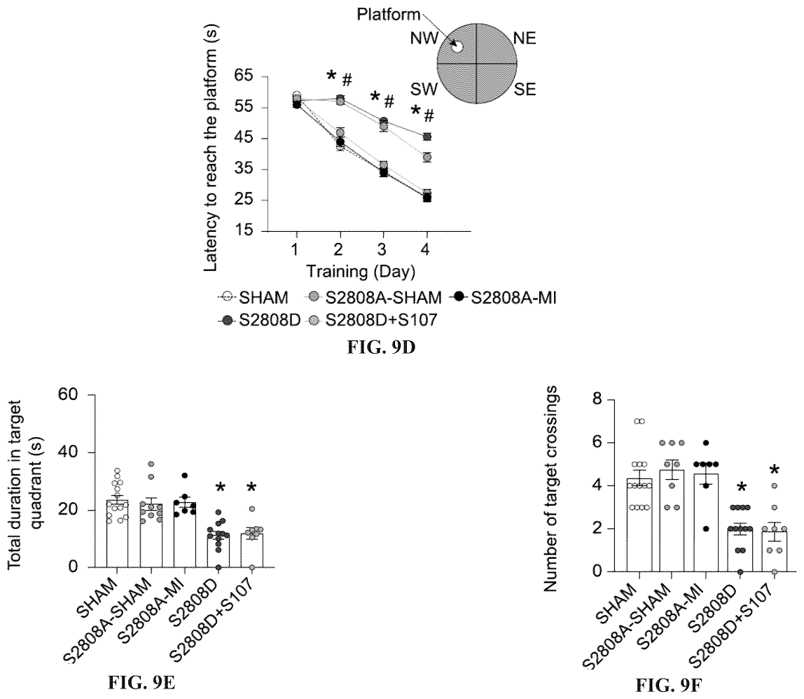

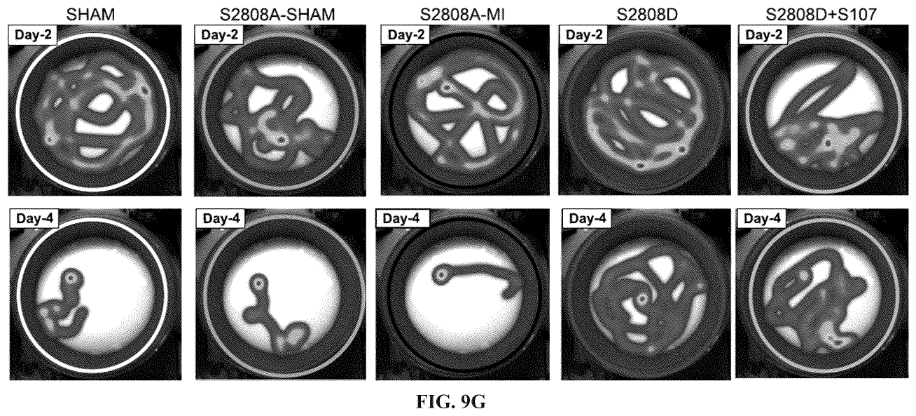

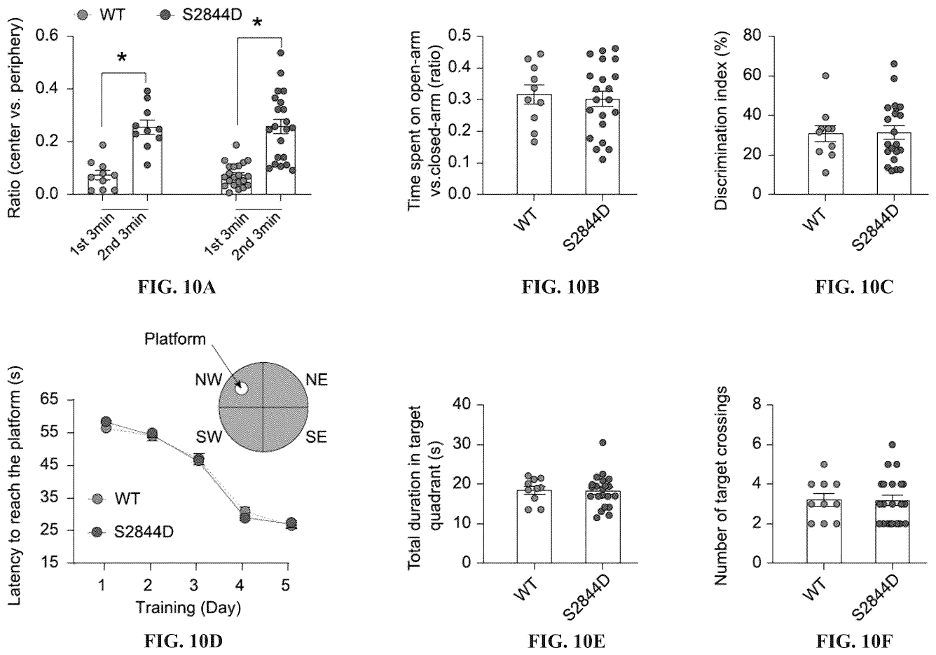

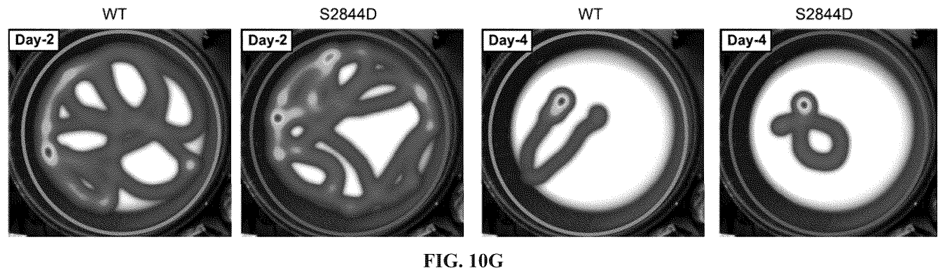

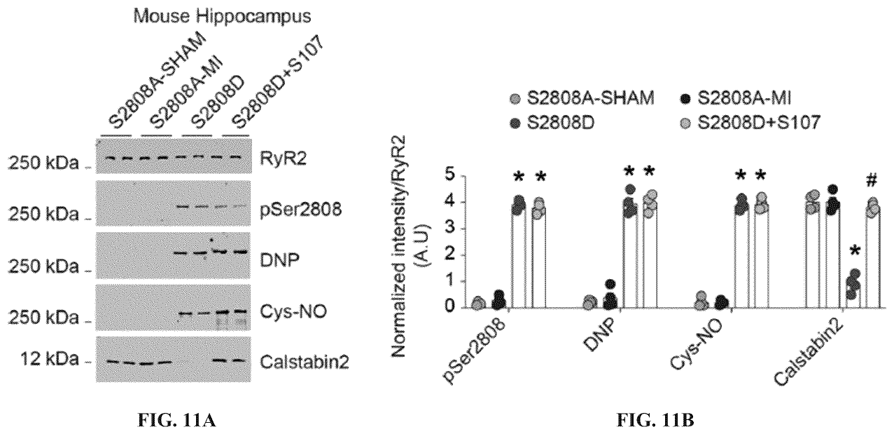

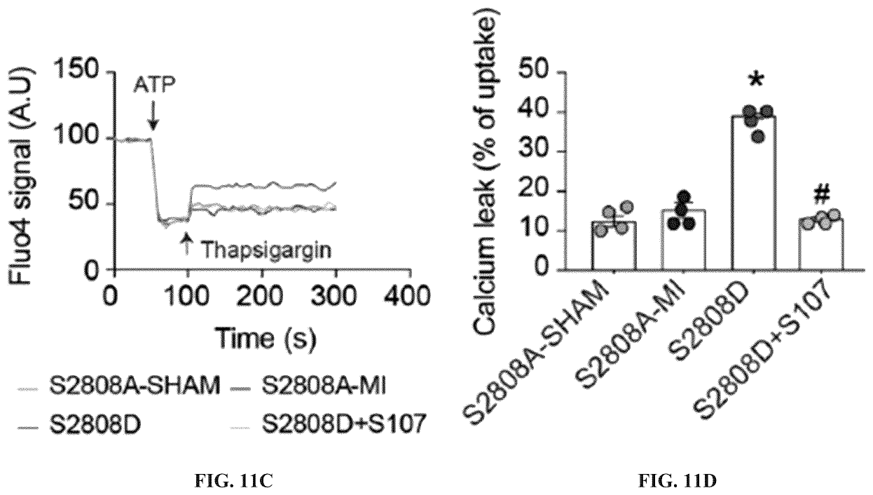

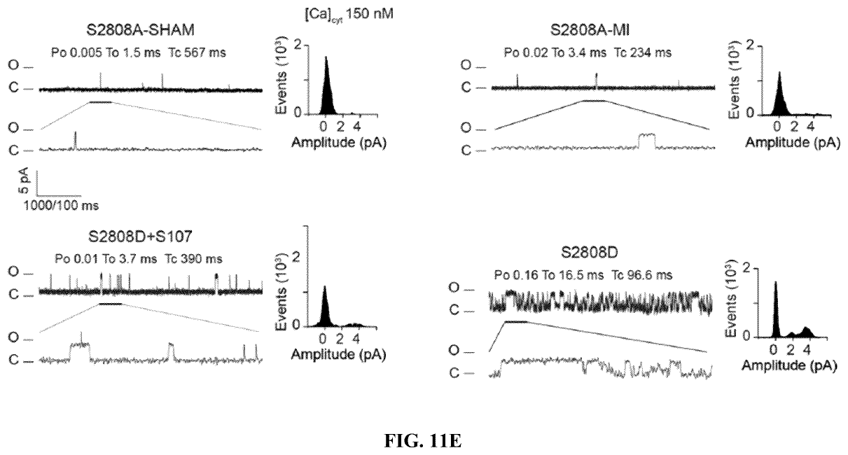

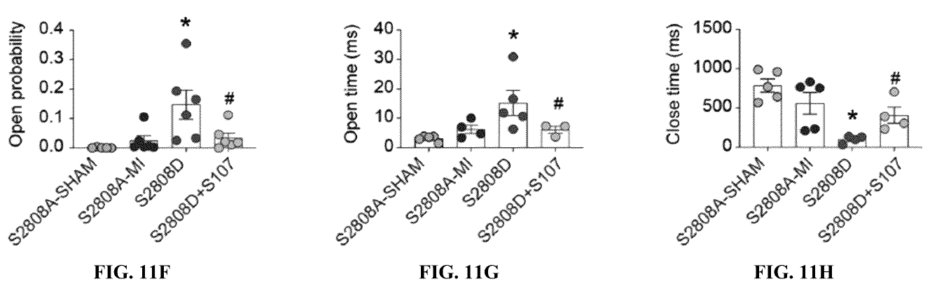

To further evaluate the specific contribution of RyR2 to CD in HF, a mouse model with RyR2 phospho-mimetic PKA phosphorylation was used on Ser2808 causing RyR2 Ca²⁺ leak (RyR2-p.Ser2808Asp) and was compared to a non-leaky RyR2 mouse model that is protected against RyR2 Ca²⁺ leak due to the genetic ablation of the RyR2 PKA-phosphorylation site Ser2808 (RyR2-p.Ser2808Ala). RyR2-p.Ser2808Ala-MI mice were protected against impaired locomotive activity and behavioral abnormalities and exhibited better learning and memory compared to RyR2-p.Ser2808Asp mice. S107 treatment improved the short-term memory and reduced the disinhibited behavior of the RyR2-p.Ser2808Asp mice (FIGS. 9A-9G). To ascertain the specific contribution of the RyR2 isoform in this process, the CD of genetically altered mice harboring a leaky RyR1 isoform (RyR1-p.Ser2844Asp) were evaluated. Cognitive impairment was not detected in these mice compared to controls (FIGS. 10A-10G). Moreover, RyR2-p.Ser2808Ala-MI were protected against RyR2 PTM and Ca²⁺ leak (FIGS. 11A-11H).

### Example 6: RyR2-Mediated Endoplasmic Reticulum Ca²⁺ Leak Upon Adrenergic Activation in Heart Failure

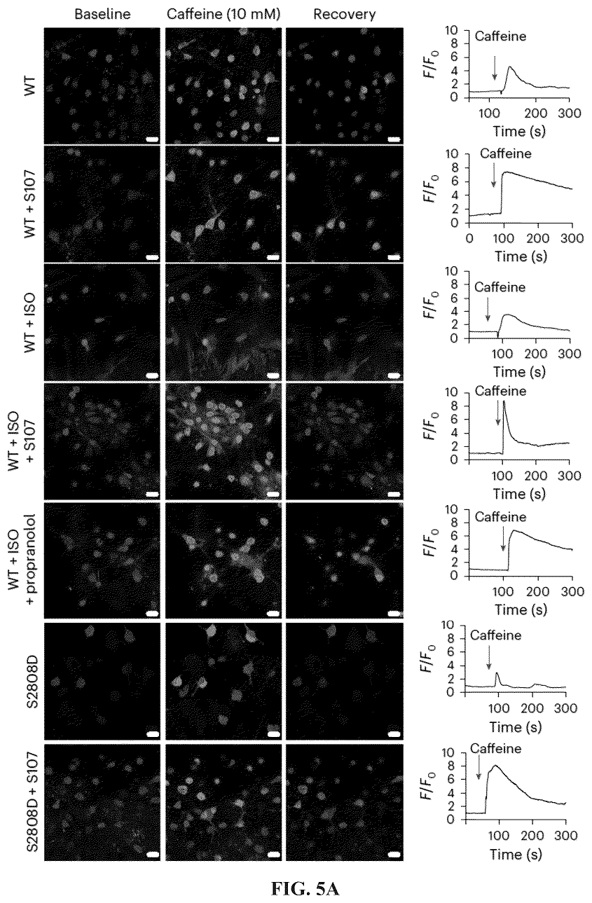

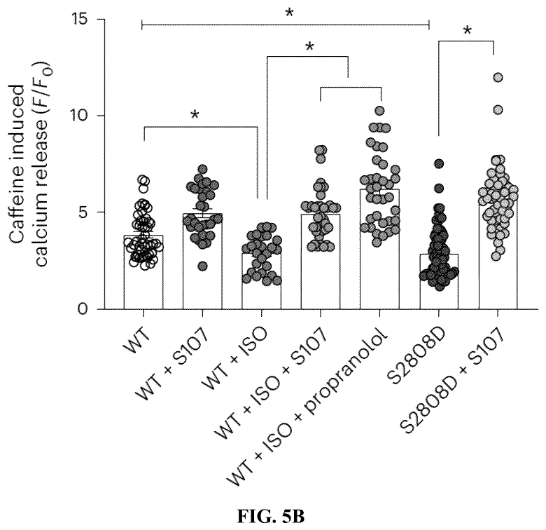

To determine whether the activation of the adrenergic pathway indeed causes neuronal RyR2-mediated ER Ca²⁺ leak, caffeine (10 mM), a well characterized RyR agonist, was used to assess RyR2 function specifically in hippocampal neurons without interference of other cell types such as glial cells or astrocytes. Indeed, caffeine application induced a small Ca²⁺ release in the hippocampal neurons treated with isoproterenol (an adrenergic agonist), which was prevented by either propranolol or S107. Moreover, hippocampal neurons expressing RyR2-p.Ser2808Asp exhibited a reduced RyR2 caffeine-induced Ca²⁺ release compared to controls. Surprisingly, S107 treatment restored caffeine-induced RyR2 Ca²⁺ release in neurons expressing RyR2-p.Ser2808Asp mutant (FIGS. 5A and 5B). These findings are in accordance with the beneficial effects of S107 and propranolol on the long-term postsynaptic potentiation, brain glucose metabolism and the cognitive function.

### Example 7: Gene Expression Changes and Neurodegenerative Pathways in Heart Failure

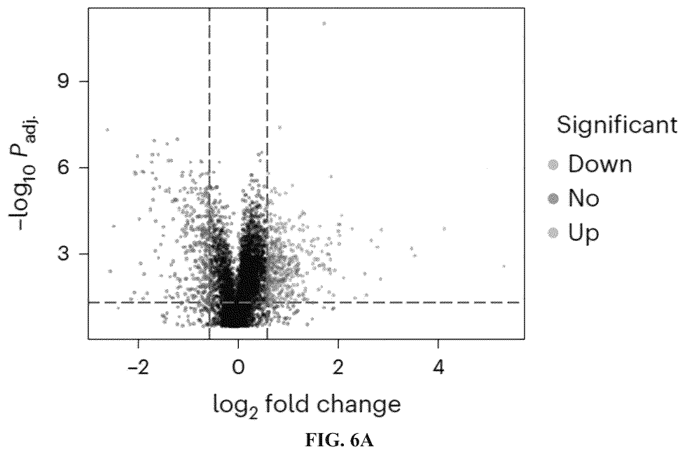

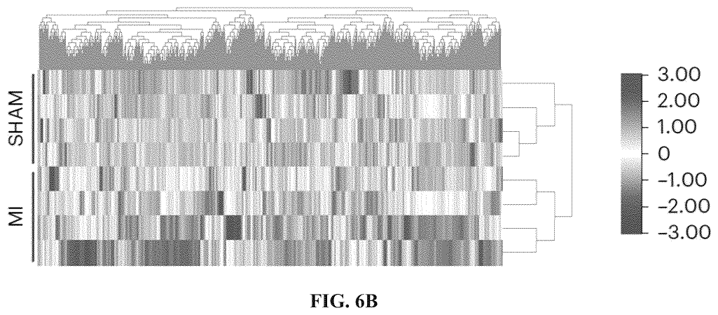

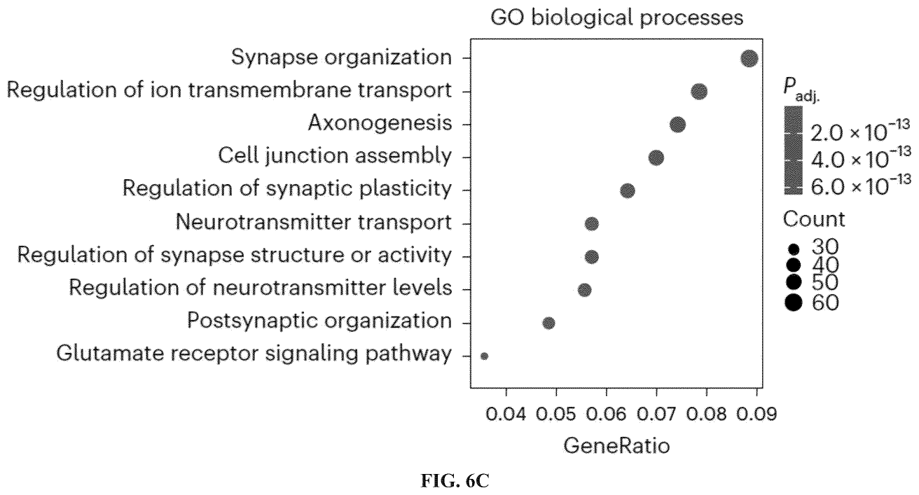

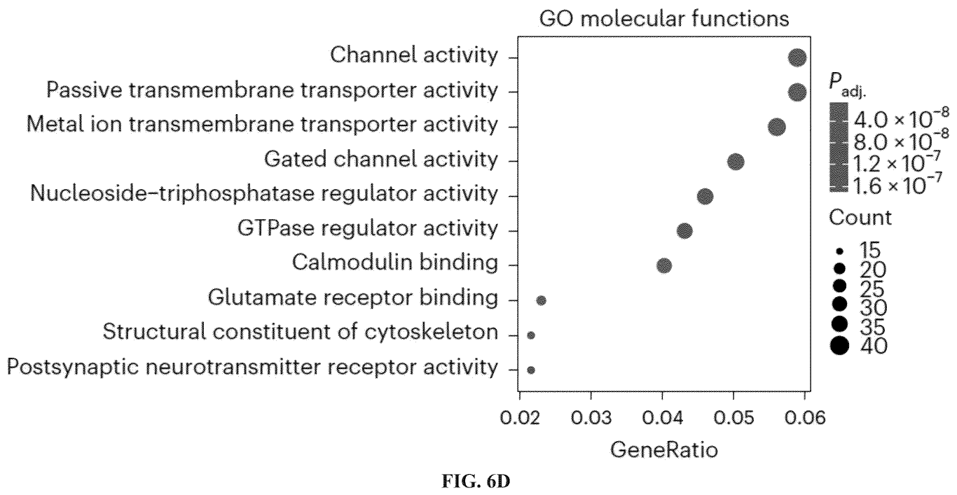

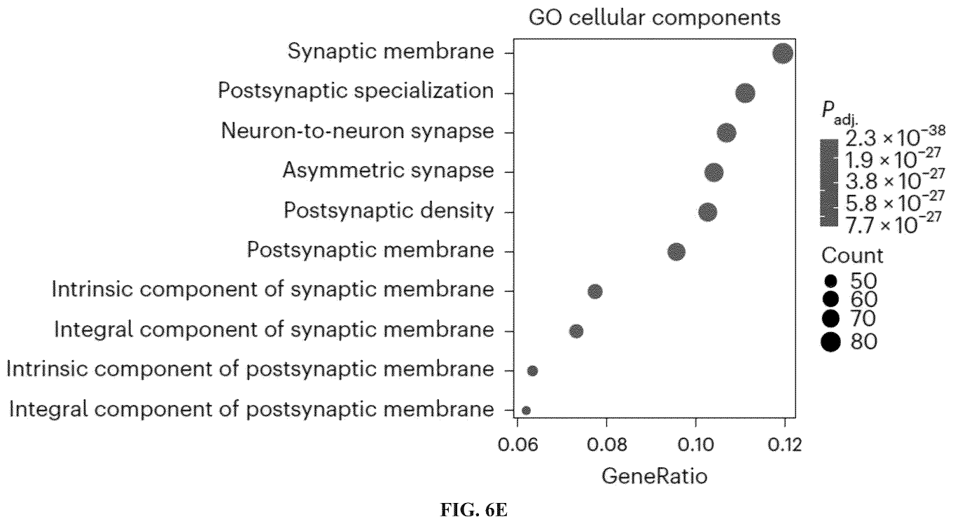

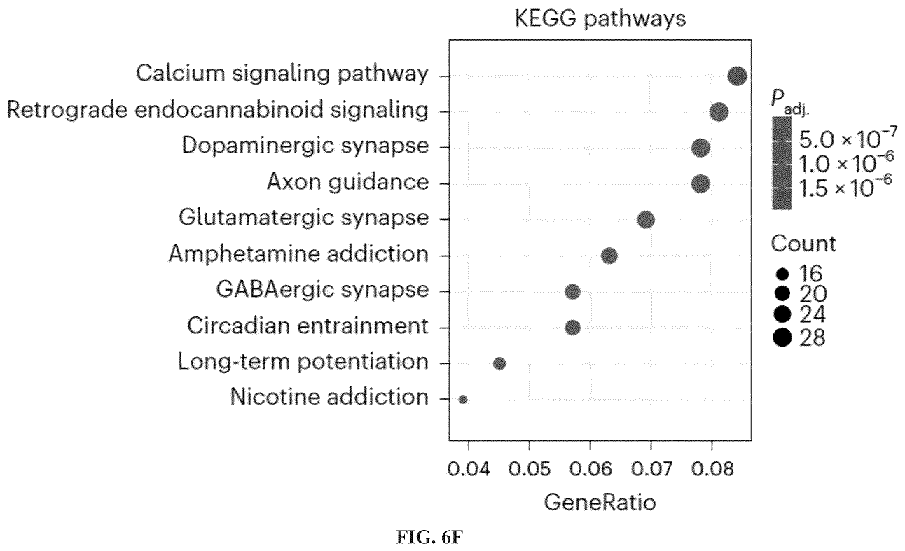

The proteome of whole-cell lysates isolated from hippocampi of MI (n=4) and SHAM (n=4) mice were evaluated. 6,049 proteins were obtained with at least one unique peptide and a 1% false discovery rate (FDR). A volcano plot of all proteins is shown in FIG. 6A. Based on the criteria of adjusted P value<0.05, fold change≥1.5, and unique peptides≥2, 737 differentially expressed unique proteins (MI versus SHAM) were found. Among these, 425 proteins were upregulated and 312 were downregulated. The heat map of 737 differentially expressed proteins is shown in FIG. 6B. The MI and SHAM groups were separately clustered, and the four replicates of each group showed good reproducibility. Gene Ontology (GO) enrichment analyses were performed on these changed proteins. The top ten significant GO terms for biological processes, molecular functions and cellular components are shown in FIGS. 6C-6E. The biological process GO analysis shows that the differentially expressed proteins were enriched for the following terms: synaptic organization, ion and neurotransmitter transport, synapse activity and plasticity (FIG. 6C). The molecular functions of these dysregulated proteins are mainly related to ion channel activity, transporter activity, GTPase regulator activity, and calmodulin binding (FIG. 6D). Significantly dysregulated proteins were located mainly at the synaptic membrane (FIG. 6E). KEGG (Kyoto Encyclopedia of Genes and Genomes) pathways were analyzed to find significant enrichment of Ca²⁺ signaling pathways (FIG. 6F). These findings are in accordance with the defective RyR2 Ca²⁺ handling and CD that were observed in MI mice.

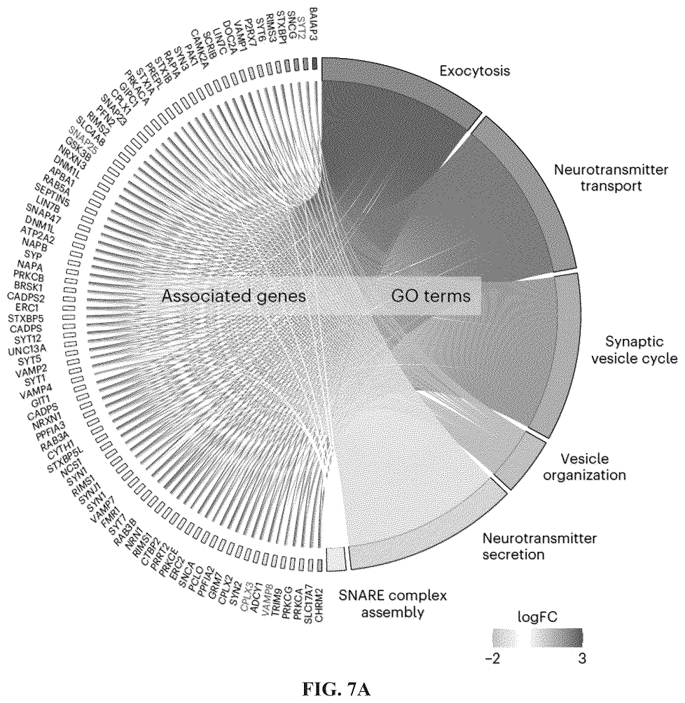

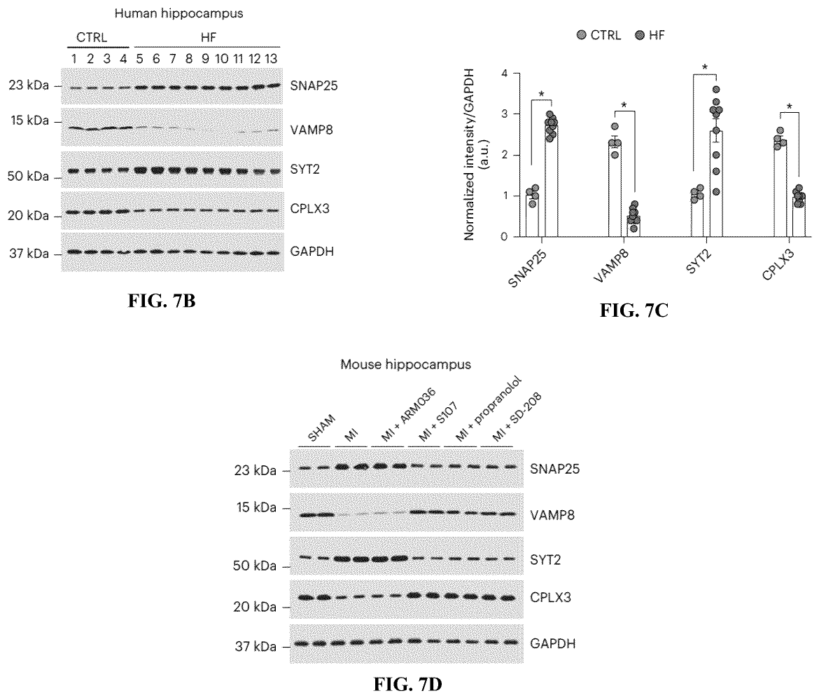

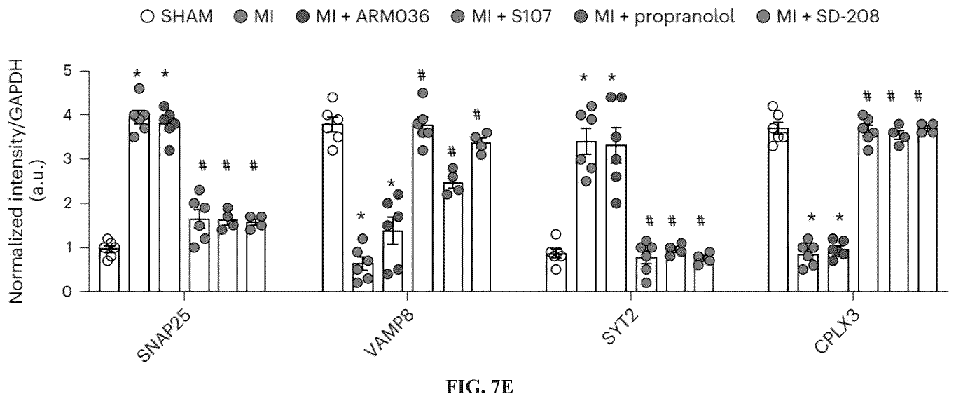

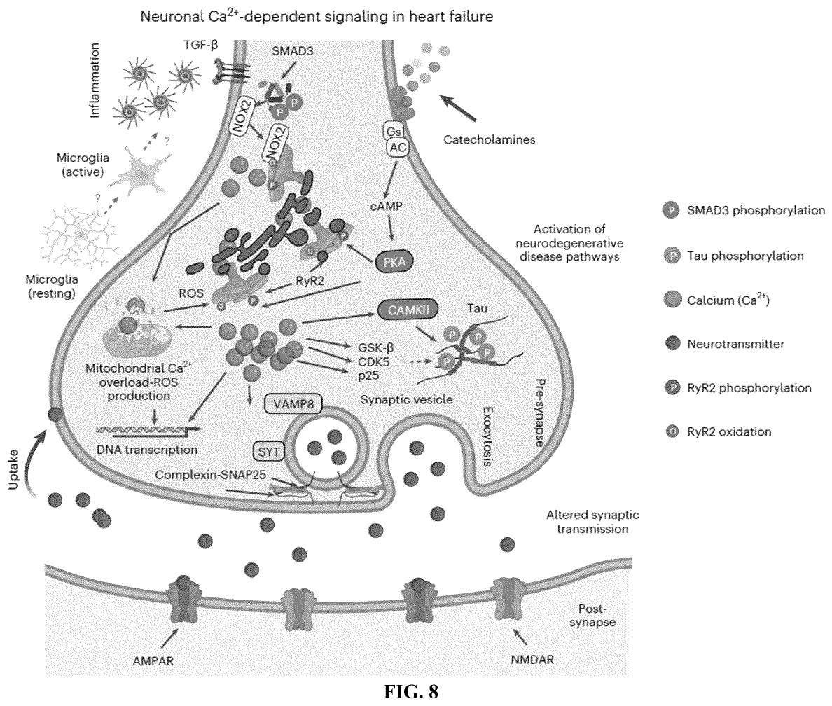

Based on the proteome data in the setting of defective Ca²⁺ regulation, four proteins were selected for further study. These four proteins were from the most enriched synaptic transmission pathways that are regulated by Ca²⁺, located near the synaptic membranes, involved in learning and memory process, and involved in neurotransmission as potential downstream signals of leaky RyR2. These selections included synaptosomal-associated protein 25 (SNAP25), vesicle-associated membrane protein 8 (VAMP8), synaptogamin-2 (SYT2) and complexin3 (CPLX3). We analyzed their protein expression levels by immunoblot in individuals with HF, and in SHAM and MI mice with or without each of the aforementioned treatments. Decreased VAMP8 and CPLX3 expression were found in hippocampi of individuals with HF and MI mice compared to SHAM mice, while SNAP25 and SYT2 expression were increased, confirming proposed proteomic analyses. These data indicate a potential role for the SNARE signaling pathway in HF⁵⁵. S107, propranolol and SD-208 treatment, but not ARM036, restored the expression of these proteins to the control levels (P<0.05, n=4-9 per group; FIGS. 7A-7E).

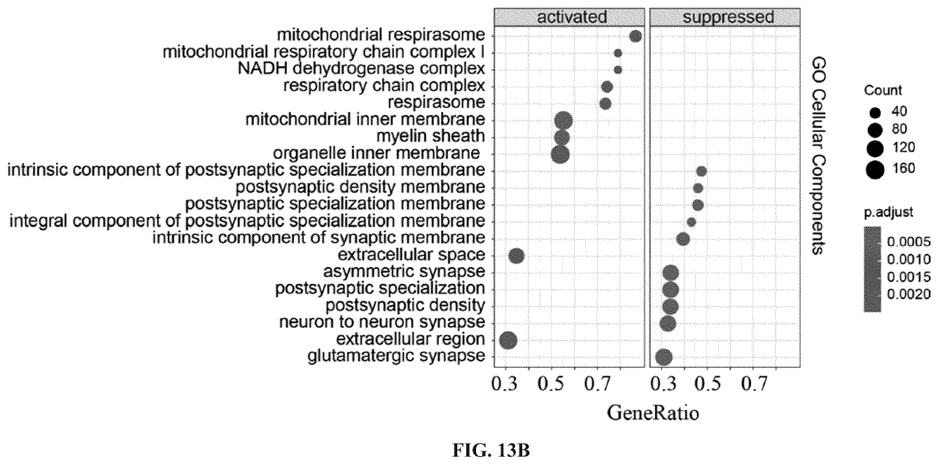

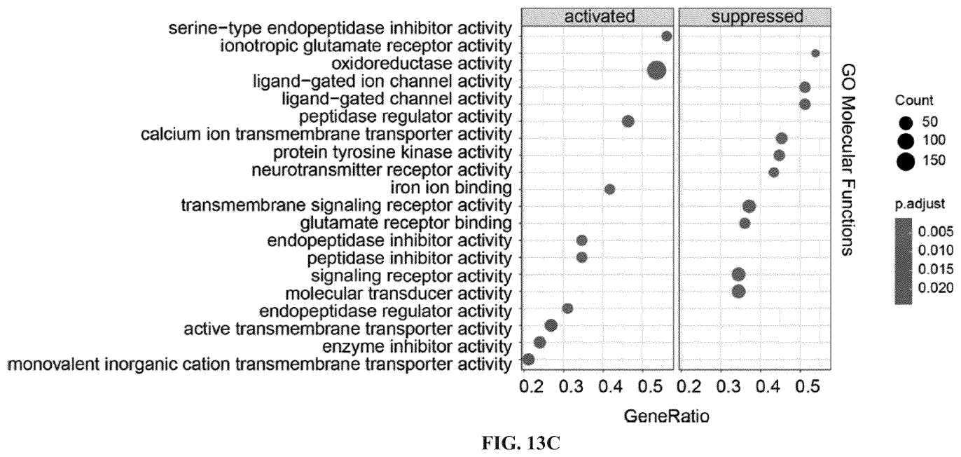

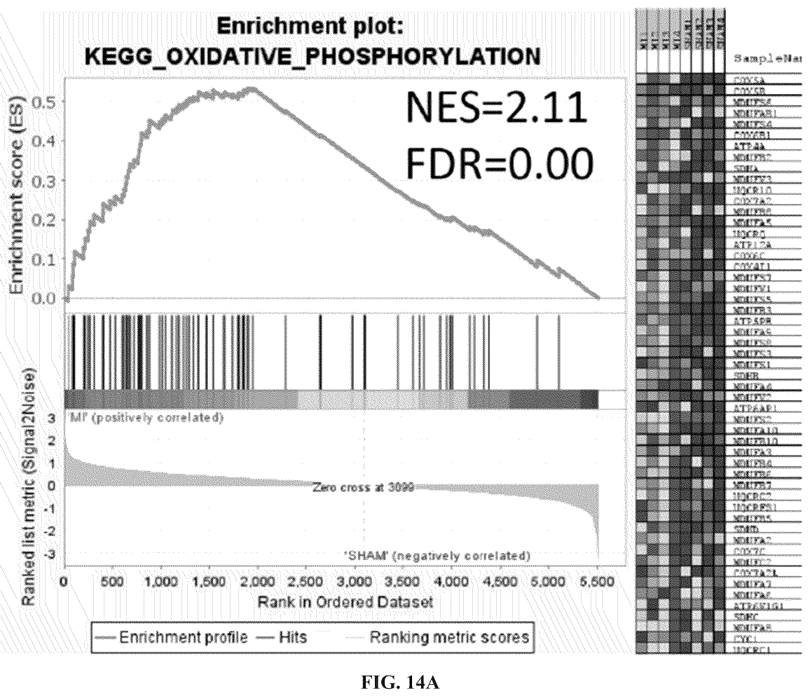

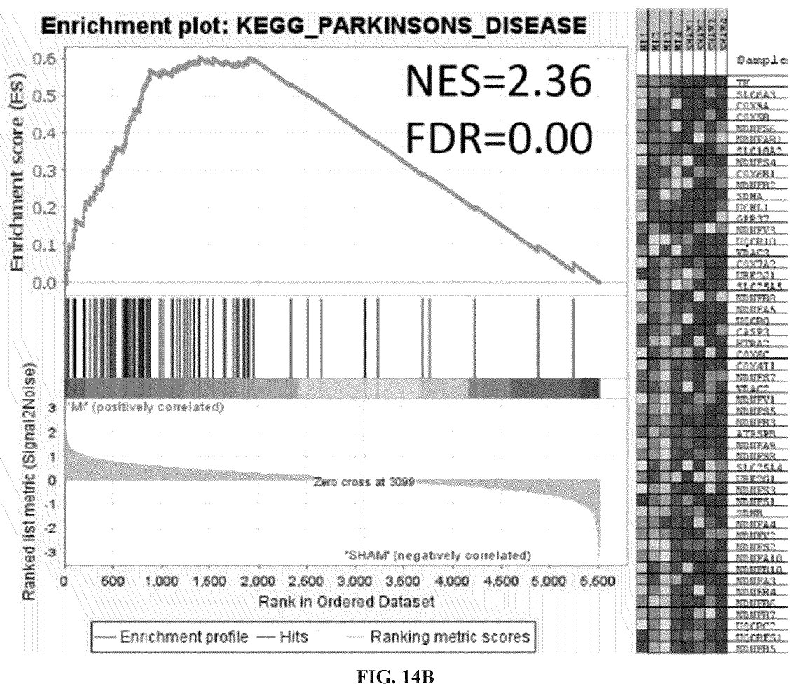

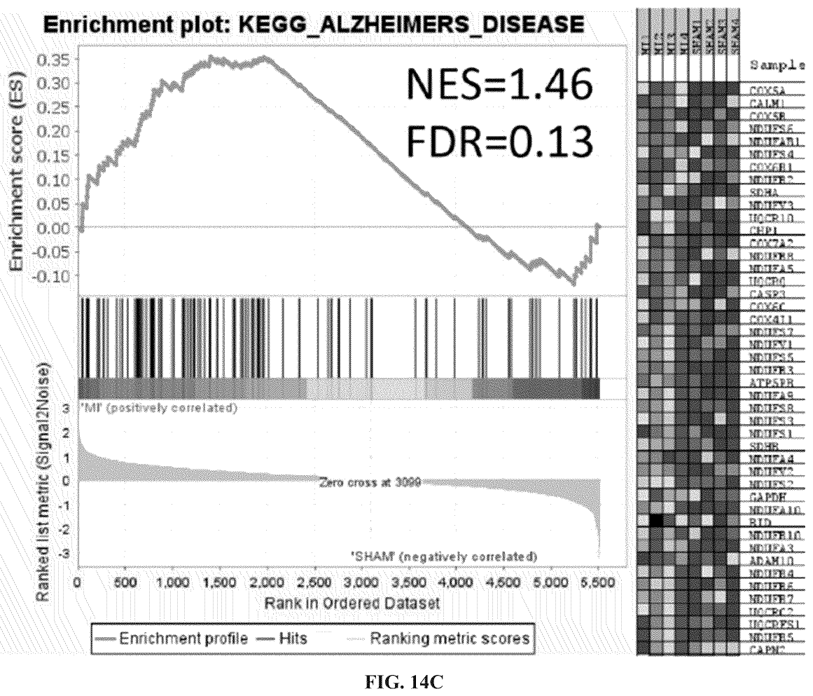

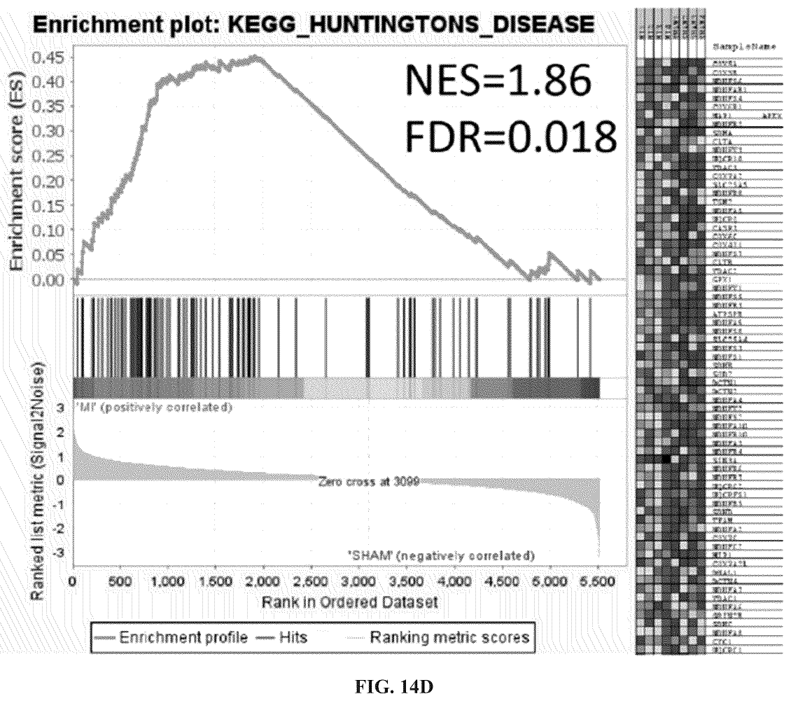

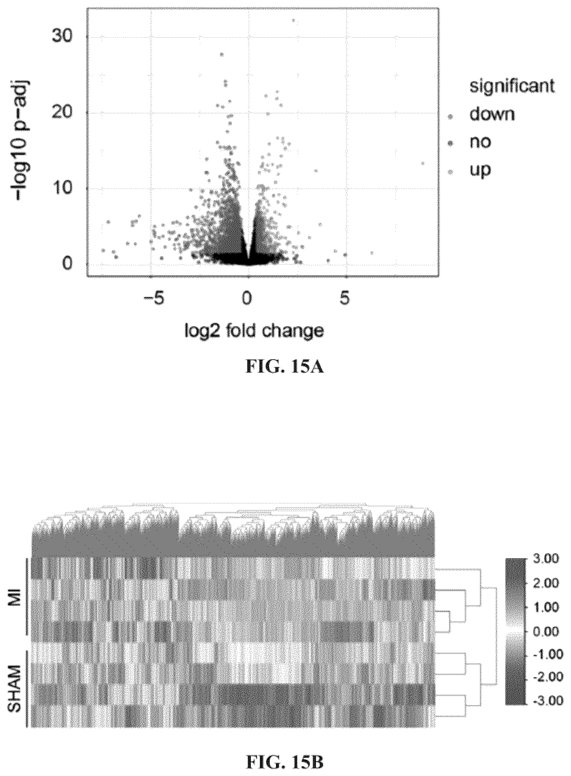

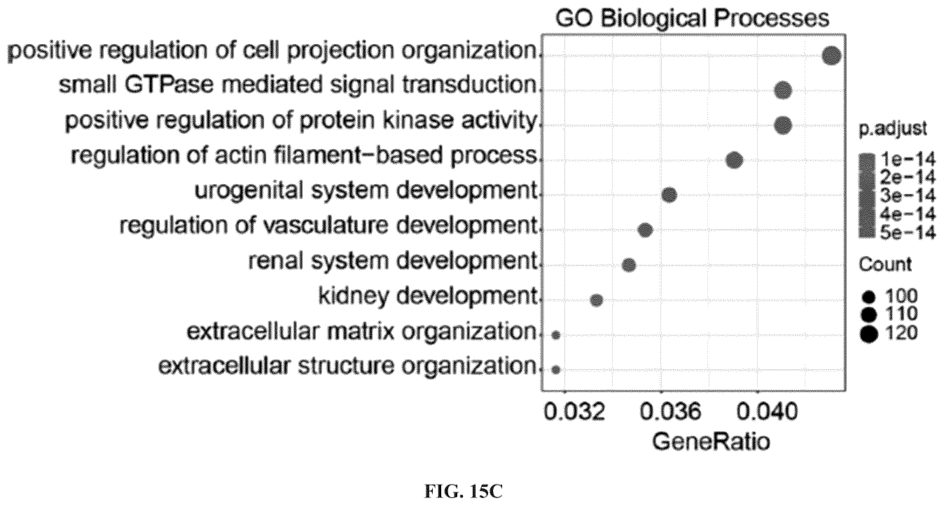

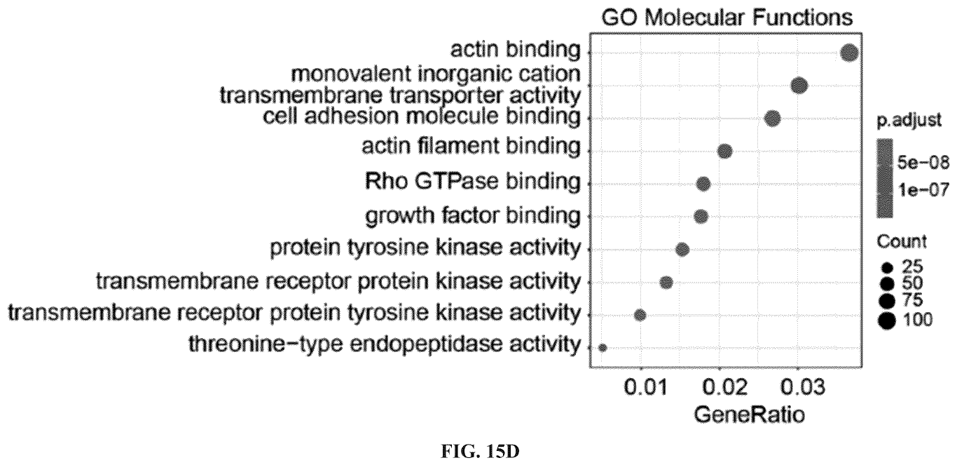

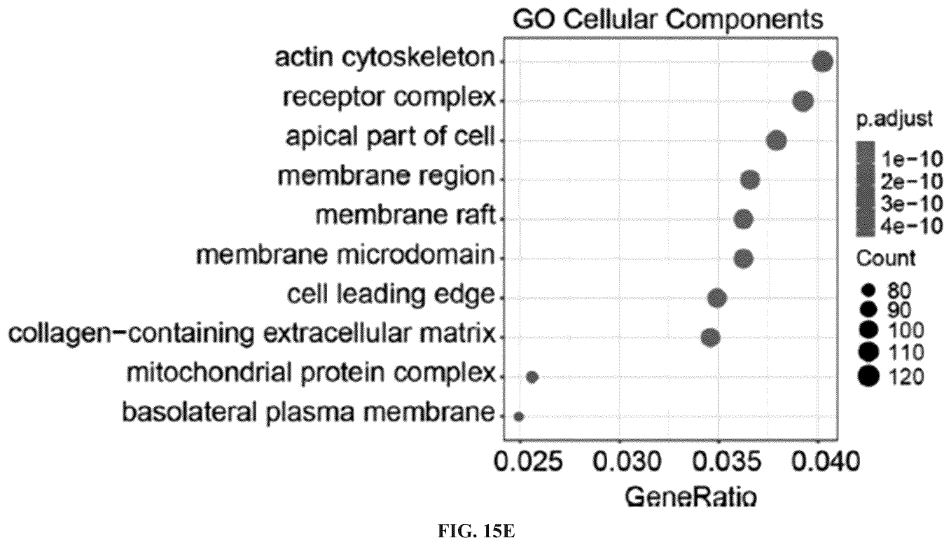

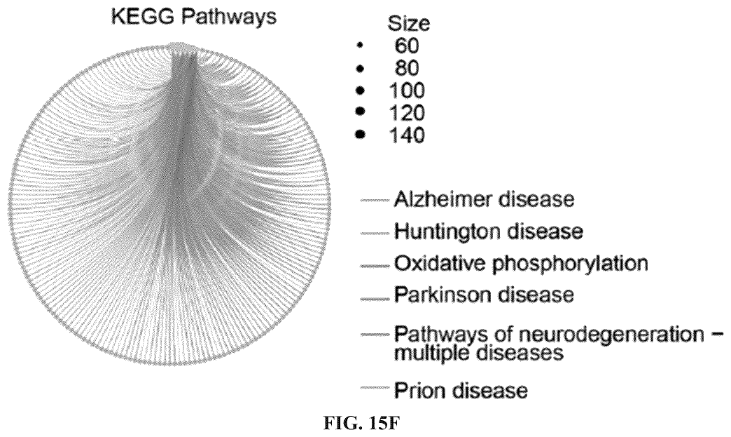

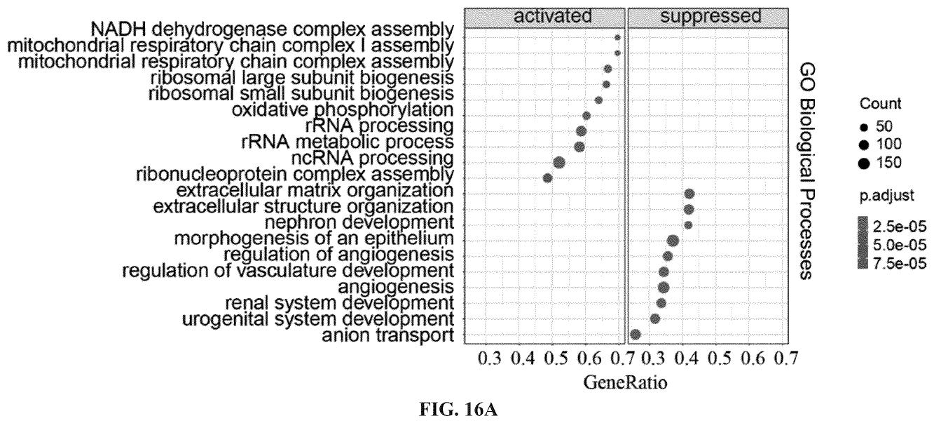

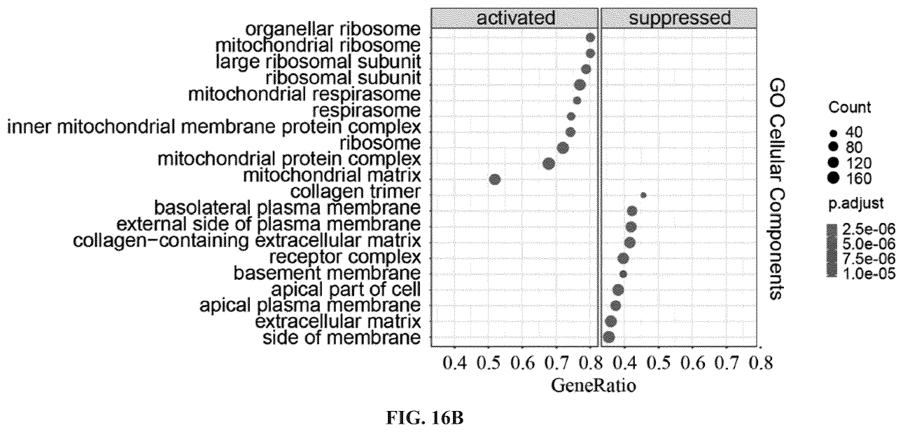

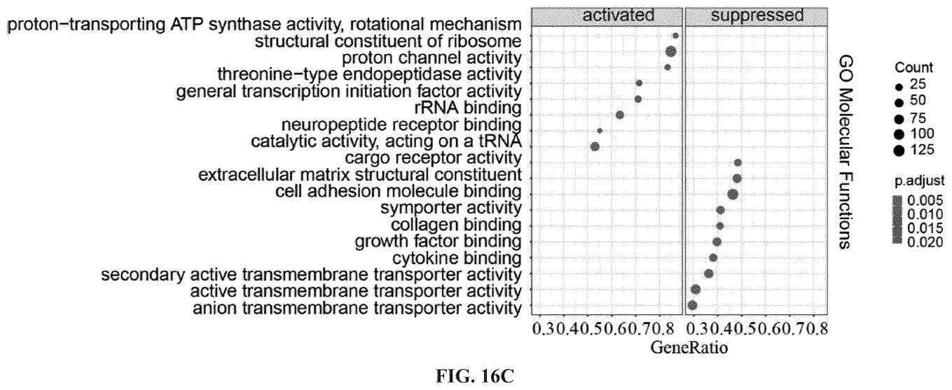

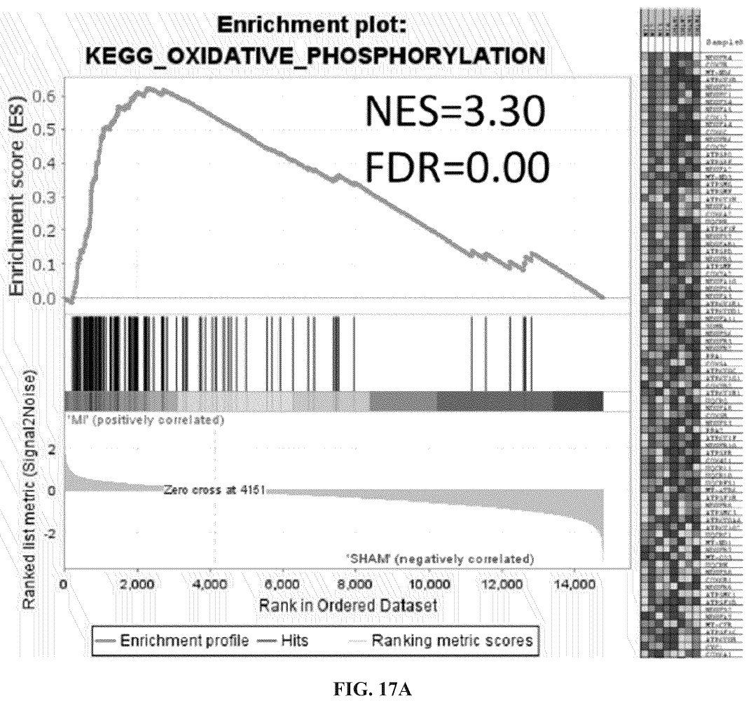

Next, GSEA was performed with the canonical pathway and GO gene sets. The top 20 (activated and suppressed) significant GO terms for biological processes, molecular function and cellular components are shown in FIGS. 13A-13C. Interestingly, there was enrichment of GO terms related to mitochondria and neurotransmission as well as oxidative phosphorylation pathways (FIGS. 13A-14C and 14A), which is potentially due to mitochondrial Ca²⁺ overload, which would stimulate the activity of the tricarboxylic acid cycle and enhance the respiratory chain complex activity by providing more substrates. Significant enrichment of neurodegenerative disease pathways were also observed, including those for Alzheimer's disease, Huntington's disease and Parkinson's disease (FIGS. 14B through 14D). These GO terms for oxidative phosphorylation defects, Parkinson's, Alzheimer's and Huntington's diseases detected by proteomics were also found at the RNA level (by RNA-sequencing analyses; FIGS. 15A-17D).

### Example 8: Upstream Signaling of Leaky Hippocampal RyR2

The hippocampal levels of norepinephrine and PKA activity were measured. Hippocampal norepinephrine levels and PKA activity were increased in MI and RyR2-p.Ser2808Asp mice (P<0.05, n=3 in each group, in line with their respective reduced cardiac function. Interestingly, RyR2-p.Ser2808Ala-MI mice showed normal norepinephrine and PKA activity comparable to the RyR2-p.Ser2808Ala-SHAM mice. S107 and ARM036 treatments had no effect on brain norepinephrine nor PKA activity compared to untreated MI mice (P=0.9, n=3), because they act directly on RyR channels not on components of the adrenergic pathway.

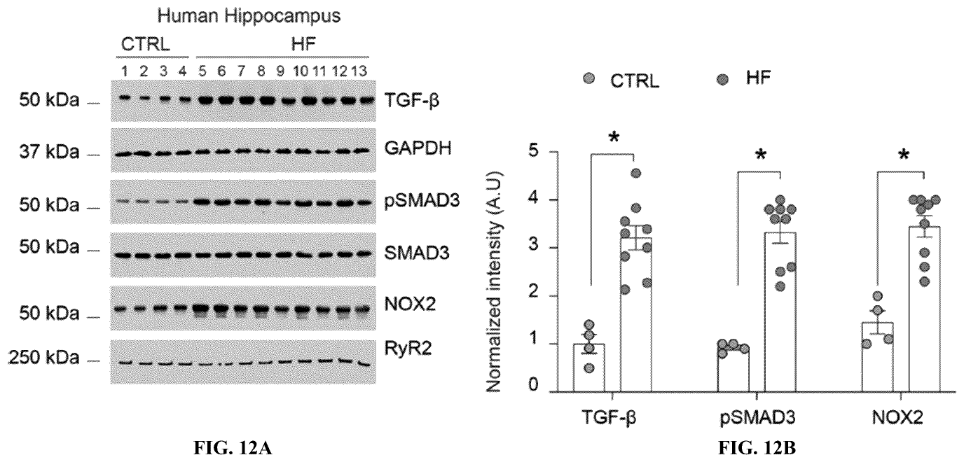

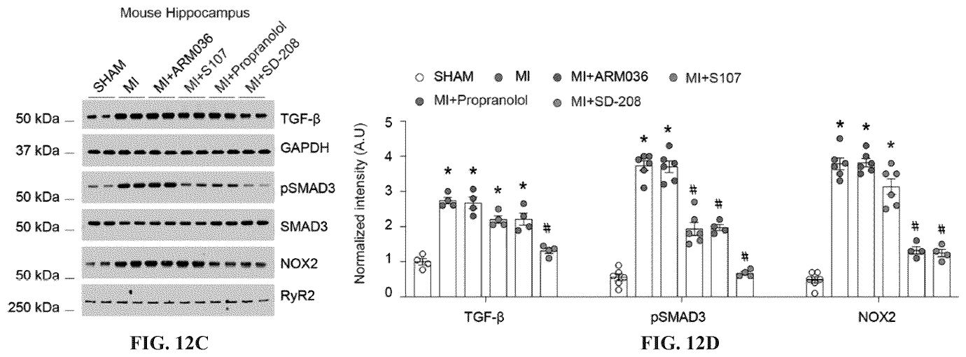

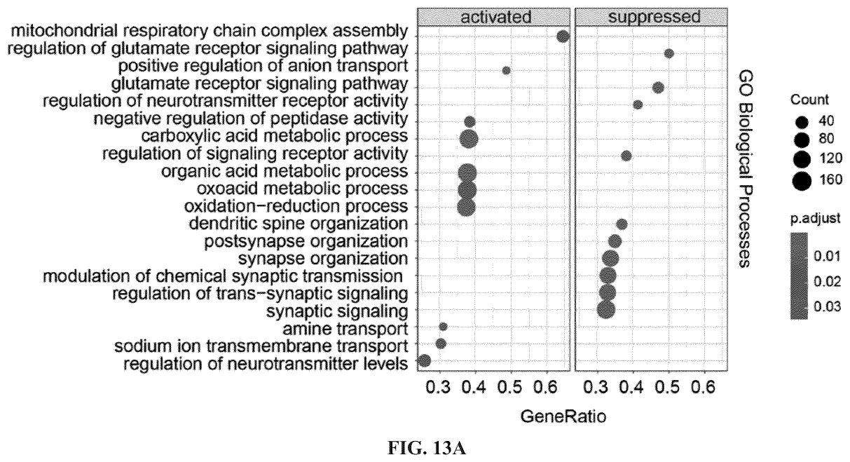

TGF-β has been implicated in CNS disorders including Alzheimer's disease. In accordance with the proteomic data, TGF-β levels and phosphorylated SMAD3 (a downstream signal of TGF-β) were increased in hippocampal tissues of individuals with HF and MI mice (P<0.05). Furthermore, NADPH oxidase 2 (NOX2) binding to hippocampal RyR2 was increased in both HF individuals and MI mice, which may account for the oxidation of RyR2 channels and ER Ca²⁺ leak in accordance with previous findings (FIG. 12). TGF-β levels, phosphorylation of SMAD3 and NOX2 binding to hippocampal RyR2 were significantly reduced by propranolol and SD-208 treatment in line with the reduced RyR2 oxidation and phosphorylation levels shown in FIGS. 3B and 3C. S107 only diminished the SMAD3 phosphorylation, whereas ARM036 had no effect on any of these changes (FIGS. 12A-12D).

### Example 9: Mitochondrial Ca²⁺ and Oxidative Overload in Heart Failure

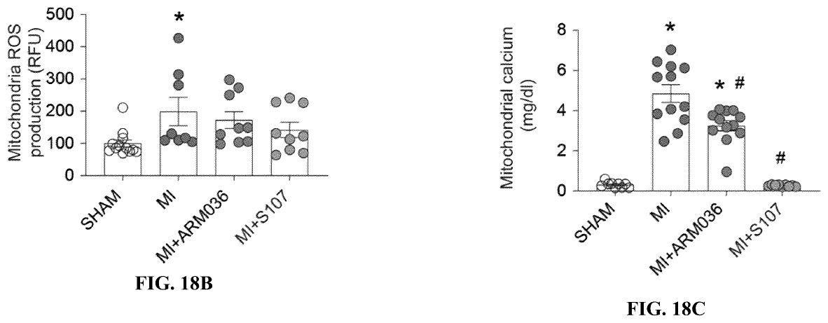

The mitochondrial Ca²⁺ content and reactive oxygen species (ROS) production were measured and the expression and PTMs (phosphorylation) of Ca²⁺ activated enzymes that have been shown to be involved in neurodegenerative diseases such as Alzheimer's disease. Our cohort plot of differentially expressed mitochondrial proteins shows an increase of the mitochondrial Rho GTPase 1 (RHOT1) in the MI mice (FIG. 18A), which is a mitochondrial GTPase involved in mitochondrial fission during high Ca²⁺ conditions 61. In line with the increased RyR2 Ca²⁺ leak, there was increased mitochondrial Ca²⁺ accumulation in MI mice (P<0.05, n=12). Interestingly, mitochondrial Ca²⁺ levels were significantly reduced by S107 treatment (P<0.05, n=9), indicating that leaky RyR2 channels are an upstream event of the mitochondrial Ca²⁺ overload (FIG. 18B). Mitochondrial ROS production was significantly increased in the MI mice (n=9) compared to the SHAM-operated group (n=12, P<0.05; FIG. 18C). Although the mitochondrial ROS production was attenuated by S107, it did not reach statistical significance (P=0.2), despite the reduction of the mitochondrial Ca²⁺ content. This is potentially due to irreversible damage that targets the electron transport chain that subsequently has led to increased electron transport chain protein expression levels observed in our proteomic analysis as a compensatory mechanism (FIGS. 13 and 14).

### Example 10: Alzheimer's Disease-Like Signaling in Heart Failure

Abnormal Ca²⁺ regulation can contribute to the activation of Ca²⁺-dependent enzymes such as AMP-activated protein kinase (AMPK), cyclin-dependent kinase 5 (CDK5) and enhanced calpain activity. Activation of these enzymes in response to elevated cytosolic Ca²⁺ levels are upstream signals of both pTau and amyloid deposits in Alzheimer's disease brains and could be a major factor in the CD observed in HF. Hippocampal samples from both HF individuals and MI mice showed increased AMPK and GSK3B phosphorylation (on Thr216) and CaMKII activity. compared to controls (P<0.05). Interestingly, phosphorylation levels of Tau on Ser199/202/262 and Thr205 were significantly increased in both HF individuals and MI mice (P<0.05). We also observed an increase in p25 expression, the neurotoxic activator of CDK5, which plays an important role in amyloid precursor protein processing in AD. Subsequently, the amyloid beta pathway may be activated, as BACEI and βCTF levels were significantly increased in individuals with HF and MI mice (FIGS. 9A-10G and 10A-10G). Finally, all of these changes were prevented/attenuated by treatment with S107, propranolol or SD-208 (P<0.05, n=4), but not by ARM036 (FIGS. 10A-10G).

### Discussion of Results

Findings of the disclosure include: (1) hippocampal neurons in HF have leaky RyR2, which correlates with behavioral abnormalities; (2) RyR2 channels are leaky due to stress-induced phosphorylation, oxidation, nitrosylation and depletion of the stabilizing subunit calstabin2; (3) hyper-adrenergic signaling and activation of TGF-β signaling are upstream signals of leaky RyR2; (4) excessive RyR2 Ca²⁺ leak is associated with mitochondrial dysfunction, impaired synaptic transmission and increased Tau pathway activation similar to that observed in Alzheimer's disease; (5) Rycal® drugs and in particular Rycal® S107 prevent loss of the RyR2 stabilizing subunit calstabin2 and rescues the aforementioned subcellular events.

The binding site for the Rycal® S107 in RyR1 and Compound 1 in RyR2 have been identified using cryogenic electron microscopy (see Melville, Z. et al. A drug and ATP binding site in type 1 ryanodine receptor. Structure 30, 1025-1034 (2022) and Miotto, M. C. et al. Structural analyses of human ryanodine receptor type 2 channels reveal the mechanisms for sudden cardiac death and treatment. Sci. Adv. 8, eabo1272 (2022). This data has provided insights into the mechanism of action by these drugs on leaky channels. Rycal® drugs bind to a cleft in the RY1&2 domain of the channel where they stabilize interactions of key residues and reduce the flexibility between domains of the cytoplasmic shell, bringing the overall channel conformation from a primed state that is easily activated closer to the closed state thus preventing aberrant ER Ca²⁺ leak. In human and experimental models of HF, increased activity of the sympathetic nervous system results in increased systemic catecholamine levels, that is, norepinephrine, an activator ligand of the adrenergic pathway. The improvement of the cognitive function and neuronal activity in the propranolol-treated MI mice, clearly shows that adrenergic signaling is an important component of cardiogenic dementia.

Of note, this neurohormonal dysregulation that affects Ca²⁺ homeostasis is not limited to the heart and the brain of HF patients, but also impacts other organs, including skeletal muscle, and accounts for chronic fatigue, reduced exercise capacity and respiratory muscle weakness. Indeed, RyR1 remodeling in skeletal muscle of HF individuals, which impairs their exercise tolerance, has also been previously reported.

Elevated brain norepinephrine levels and increased PKA activity associated with RyR2 Ca²⁺ leak in mice with failing hearts were also observed. This biochemical signature and dysfunction of RyR2 were correlated with behavioral abnormalities. TGF-6, an upstream mediator of RyR2 oxidation via NOX2 enzymes, is also increased, thereby accounting for further oxidation of RyR channels and increased SR/ER Ca²⁺ leak in HF. In line with previously published studies, TGF-β inhibitor SD-208 significantly reduced RyR2 oxidation, rebound calstabin2 to the channel and decreased ER Ca²⁺ leak in the MI mice. Moreover, SD-208 improved LTP, brain glucose metabolism and the overall cognitive function of MI mice. Although the TGF-β family inhibitors have shown relative efficacy as an anticancer treatment, the SD-208 compound has rarely been tested for its effects on cognition. For instance, SD-208 was able to inhibit germinal matrix hemorrhage in a rat model of the disease with a partial recovery of the motor and cognitive function. SD-208 treatment improved the spatial learning in a rat model of HIV-1-associated neurocognitive disorders. Of note, this TGF-β inhibitor was used to dissect the cellular mechanisms of cardiogenic dementia, but its potential as a drug candidate remains uncertain because of its cardiac toxicity and the side effects of blocking the physiological TGF-β signaling in healthy cells.

It has been found that a beneficial effect of propranolol on cognition and memory in the context of cardiogenic dementia was observed, with this in line with previously published studies showing improvement of cognitive function with beta-blocker therapy in different contexts such as hypertensive older individuals and patients Alzheimer's disease. Interestingly, propranolol displayed beneficial effects on cognition, especially on sustaining cognitive performance over time in healthy individuals, and reduced amyloid and Tau pathology in Alzheimer's transgenic mice. Other studies, however, have reported deleterious effects of beta blockers on cognitive function in patients after MI and in the general population. Because propranolol is extensively used to treat HF patients, a randomized clinical trial is necessary to resolve this controversy.

Mitochondrial Ca²⁺ accumulation, a downstream effect of TGF-β and leaky RyR2 channels was enhanced in MI mice, with significant upregulation of mitochondrial ROS production. These changes trigger a vicious cycle in which RyR2 leak leads to mitochondrial Ca²⁺ overload, which then produces ROS and further oxidizes RyR2 channels. Such a vicious cycle of Ca²⁺/ROS alteration could lead to gene and/or protein expression abnormalities that would worsen the prognosis of HF patients over time. The deep changes in the transcriptome and the proteome in the MI mice along with the epigenetic alterations we previously reported in the RyR2-p.Ser2808Asp mice serve as preliminary evidence of this hypothesis. Logically, oxidative stress and Ca²⁺ dysregulation would initiate a cellular response that would manifest at the level of neuronal gene expression. Support for this view stems from the observation of upregulation of 425 and downregulation of 312 proteins and genes in hippocampi from MI mice. Interestingly, the expression of key proteins regulated by Ca²⁺ and involved in synaptic transmission were modified including CPLX3, as well as SNAP25, SYT2 and VAMP8, which may explain, in part, the CD observed in our MI and RyR2-p.Ser2808Asp mice.

For example, Ca²⁺ triggers rapid exocytosis of neurotransmitters from neurons. Such a process is mediated by synaptogamins, an abundant component of synaptic vesicles that binds Ca²⁺ ions through two C₂ domains. The present data reveals a significant increase of the SYT2 isoform and SNAP25 at the protein expression level, which is an indicator of defective Ca²⁺-dependent exocytosis and altered hippocampal synaptic transmission in HF in line with the decreased LTP and brain glucose metabolism in the MI mice. Furthermore, the changes in protein expression parallel some of the changes observed in models of neurodegenerative diseases. Postmortem studies of Alzheimer's disease brains have shown altered levels of several synaptic proteins, including SNAP25 and synaptogamins, components of the SNARE complex. Furthermore, cerebrospinal fluid (CSF) levels of SNAP25 and synaptogamins have been assessed and found to be elevated in patients with Alzheimer's disease or middle cognitive impairment, compared to controls. This appears to be the first study reporting alterations in the hippocampal synaptic proteins and cognitive impairment in HF and providing a unique transcriptome and proteome library for the future mechanistic investigations and supported by in vivo and in vitro cognitive testing. This is particularly important for the initiation of large-scale clinical studies to assess some of these markers in the CSF or the plasma of HF patients as a predictive molecular fingerprint to monitor the onset and the progression of cognitive impairment in these patients.

Finally, impaired hippocampal glucose metabolism were found in HF mice using [18F] FDG-PET scan imaging. This is a valuable neuroimaging tool for early detection of cardiogenic dementia and could be added as a clinical biomarker to support assessment and management of HF patients. Further studies to confirm its efficacy in early detection of CD are needed. Taken together, our data are in line with previous reports suggesting that defective Ca²⁺ plays an instrumental role in neurodegeneration and cognitive impairment. Further analyses of protein expression levels and PTMs showed significant upregulation of Ca²⁺ dependent enzymes involved in Tau processing and Alzheimer's disease. These markers were found to be increased in a small cohort of individuals with coronavirus disease 2019 suspected of developing a forme fruste of Alzheimer's disease due to defective Ca²⁺ regulation and inflammation. HF patients and MI mice exhibited increased TGF-β and SMAD3 phosphorylation levels that potentially play a role in cardiogenic dementia. Increased adrenergic activity and inflammatory pathway activation in HF primarily impairs intracellular Ca²⁺ regulation. Excessive ER Ca²⁺ leak enhances oxidative stress, dysregulates neuronal gene/protein expression and primes neurodegenerative pathways.

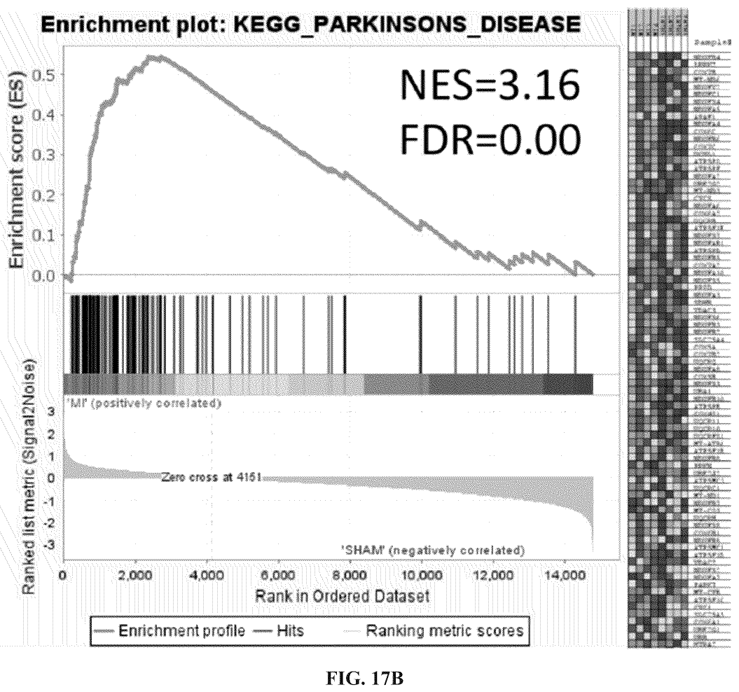

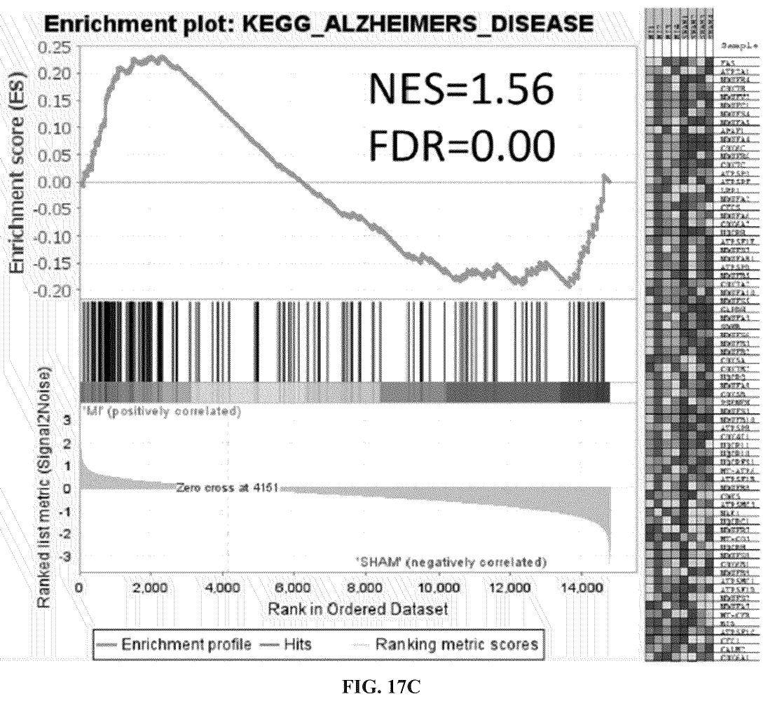

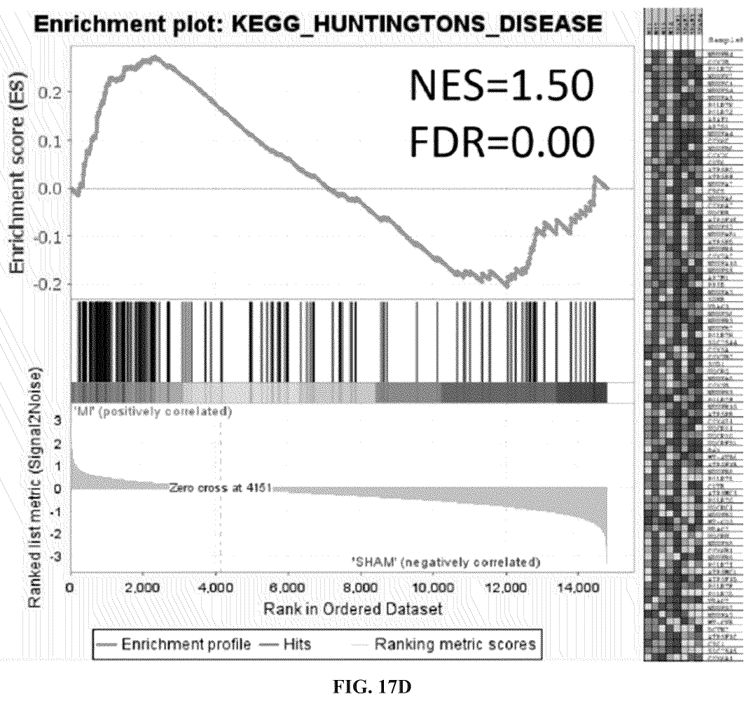

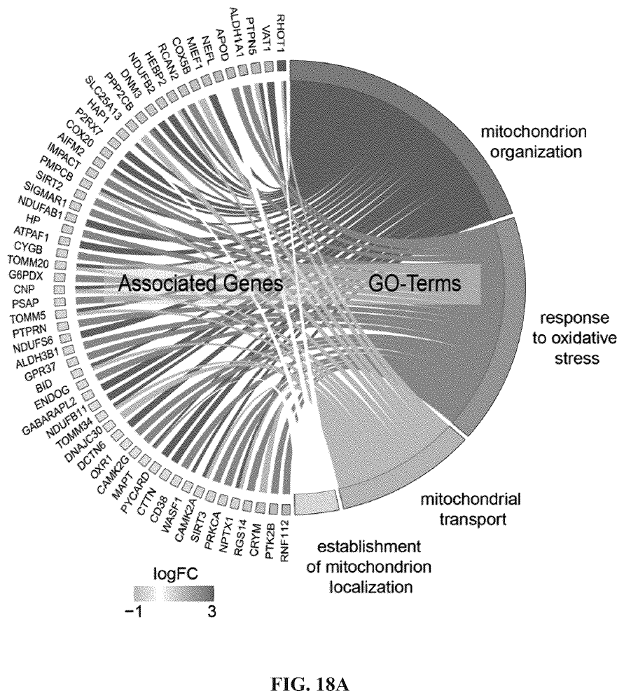

These subcellular changes impair the learning and memory processes in HF, which is detrimental for patients' compliance to medication and early recognition of worsening symptoms. These pathways are summarized in FIG. 8 which is a schematic illustration of neuronal Ca²⁺ signaling in heart failure. Increased catecholamine levels during HF activate PKA, which phosphorylates RyR2 on Ser2808 (FIGS. 3A-3G). Increased inflammation in HF includes activation of the TGF-β pathway resulting in SMAD3 phosphorylation and upregulation of NOX2 and binding to RyR2 (FIGS. 4A-4J). NOX2 promotes oxidation of RyR2 channels 58-60. The combination of oxidation and phosphorylation of RyR2 results in ER Ca²⁺ leak (FIGS. 3A-3G). Ca²⁺ leak through RyR2 leads to increased mitochondrial Ca²⁺ accumulation, which enhances mitochondrial ROS production (FIGS. 18A-18C). Therefore, a vicious cycle is created between the mitochondria and RyR2, where increased ER Ca²⁺ leak causes mitochondrial ROS production and increased mitochondrial ROS production further oxidizes RyR2 and renders it leakier. Chronic RyR2 Ca²⁺ leak depletes ER Ca²⁺ content and reduces the Ca²⁺ transient (FIGS. 5A and 5B) required for synaptic vesicle release during synaptic transmission (FIGS. 4 A-4J and 7A-7E). Furthermore, oxidative stress and Ca²⁺ dyshomeostasis alter gene transcription (FIGS. 15A-15F), with a particular effect on proteins that are regulated by Ca²⁺ and involved in neurotransmission. Dysregulation of key proteins involved in synaptic transmission is reflected in the impaired LTP observed in the MI mice (FIGS. 4B and 4C). Accumulation of Ca²⁺ in the cytosol activates Ca²⁺-dependent enzymes including CAMKII, GSK-β, CDK5 and p25, which subsequently leads to Tau phosphorylation, a hallmark of neurodegenerative disease (FIGS. 17A-17D and 18A-18C). All these activated signaling cascades can be prevented, at least in part, by S107, a Rycal® drug that reduces the ER Ca²⁺ leak. Gs, G protein; AC, adenylyl cyclase; cAMP, cyclic AMP; GSK-β, glycogen synthase kinase 3 beta.

The studies conducted for the present disclosure included a relatively small sample size of only nine individuals with HF who had incomplete clinical information. Multiple mouse models were used in order to overcome this. Moreover, all individuals with HF were younger than controls, minimizing age-related confounding factors. Other factors such as individuals' backgrounds, socioeconomic status, hospitalization history and drug use could, however, contribute to CD.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

1. A method of treating, preventing or reducing the likelihood of developing cognitive dysfunction associated with or induced by a cardiac condition, the method comprising administering a therapeutically effective amount of a calcium channel stabilizer to a subject in need thereof, the calcium channel stabilizer comprising a 1,4-benzothiazepine moiety.

2. The method of claim 1, wherein the cardiac condition is heart failure.

3. The method of claim 2, wherein the heart failure is chronic heart failure, acute heart failure, heart failure with reduced ejection fraction, heart failure with preserved ejection fraction, acute decompensated heart failure, heart failure with systolic dysfunction, or heart failure with diastolic dysfunction.

4. The method of claim 1, wherein the cardiac condition is characterized by an irregular heartbeat or an arrhythmia.

5. The method of claim 1, wherein the subject is a heart failure patient having an implantable cardioverter-defibrillator, wherein the implantable cardioverter-defibrillator is implanted in the patient.

6. The method of claim 1, wherein the cardiac condition is myocardial infarction.

7. The method of claim 1, wherein the cardiac condition comprises cardiac ischemia/reperfusion injury.

8. The method of claim 1, wherein the cognitive dysfunction results from a RyR2-mediated intracellular Ca²⁺ leak.

9. The method of claim 1, wherein the calcium channel stabilizer decreases Ca²⁺ leak from a RyR2 channel of the subject.

10. The method of claim 1, wherein the calcium channel stabilizer increases RyR2-Castabin2 binding in the subject.

11. The method of claim 1, wherein the calcium channel stabilizer decreases open probability (Pₒ) of RyR2 in the subject.

12. The method of claim 1, wherein the cognitive dysfunction comprises a deficit in attention, executive functioning, language, processing speed, learning, short term memory, long term memory, and any combination thereof.

13. The method of claim 1, wherein the calcium channel stabilizer is represented by the structural formula: 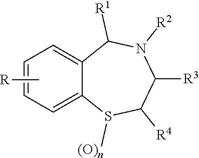
wherein,
n is 0, 1, or 2;
R is located at one or more positions on the benzene ring; each R is independently selected from the group consisting of H, halogen, —OH, —NH₂, —NO₂, —CN, —N₃, —SO₃H, acyl, alkyl, alkoxyl, alkylamino, cycloalkyl, heterocyclyl, heterocyclylalkyl, alkenyl, (hetero-)aryl, (hetero-)arylthio, and (hetero-)arylamino; wherein each acyl, alkyl, alkoxyl, alkylamino, cycloalkyl, heterocyclyl, heterocyclylalkyl, alkenyl, (hetero-)aryl, (hetero-)arylthio, and (hetero-)arylamino may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl;
R¹ is selected from the group consisting of H, oxo, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl;
R² is selected from the group consisting of —C═O(R⁵), —C═S(R⁶), —SO₂R⁷, —POR⁸R⁹, —(CH₂)ₘ—R¹⁰, alkyl, aryl, heteroaryl, cycloalkyl, cycloalkylalkyl, and heterocyclyl; wherein each alkyl, aryl, heteroaryl, cycloalkyl, cycloalkylalkyl, and heterocyclyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
R³ is selected from the group consisting of H, —CO₂Y, —CONY, acyl, alkyl, alkenyl, aryl, cycloalkyl, and heterocyclyl; wherein each acyl, alkyl, alkenyl, aryl, cycloalkyl, and heterocyclyl may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl; and wherein Y is selected from the group consisting of H, alkyl, aryl, cycloalkyl, and heterocyclyl;
R⁴ is selected from the group consisting of H, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N, —O, —S, —CN, —N₃, —SH, nitro, oxo, acyl, alkyl, alkoxyl, alkylamino, alkenyl, aryl, (hetero-)cycloalkyl, and (hetero-)cyclyl;
R⁵ is selected from the group consisting of —NR¹⁶, NHNHR¹⁶, NHOH, —OR¹⁵, —CONH₂NHR¹⁶, —CO₂R¹⁵, CONR¹⁶, —CH₂X, acyl, alkenyl, aryl, cycloalkyl, cycloalkylalkyl, heterocyclyl, and heterocyclylalkyl; wherein each acyl, alkenyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
R⁶ is selected from the group consisting of —OR¹⁵, —NHNR¹⁶, —NHOH, —NR¹⁶, —CH₂X, acyl, alkenyl, alkyl, aryl, cycloalkyl, cycloalkylalkyl, heterocyclyl, and heterocyclylalkyl; wherein each acyl, alkenyl, alkyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
R⁷ is selected from the group consisting of —OR¹⁵, —NR¹⁶, —NHNHR¹⁶, —NHOH, —CH₂X, alkyl, aryl, cycloalkyl, cycloalkylalkyl, heterocyclyl, and heterocyclylalkyl; wherein each alkyl, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
R⁸ and R⁹ independently are selected from the group consisting of OH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkyl, alkoxyl, alkylamino, alkenyl, aryl, (hetero-)cycloalkyl, and (hetero-)cyclyl;
R¹⁰ is selected from the group consisting of —NH₂, —OH, —SO₂R¹¹, —NHSO₂R¹¹, —C—O(R¹²), —NHC═O(R¹²), —OC═O(R¹²), and —POR¹³R¹⁴,
R¹¹, R¹², R¹³, and R¹⁴ independently are selected from the group consisting of H, —OH, —NH₂, —NHNH₂, —NHOH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
X is selected from the group consisting of halogen, —CN, —CO₂R¹⁵, —CONR¹⁶, —NR¹⁶, —OR¹⁵, —SO₂R⁷, and —POR⁸R⁹; and
R¹⁵ and R¹⁶ independently are selected from the group consisting of H, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N, —O, —S, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
or a pharmaceutically-acceptable salt, hydrate, solvate, complex, or prodrug thereof.

14. The method of claim 1, wherein the calcium channel stabilizer is represented by the structural formula 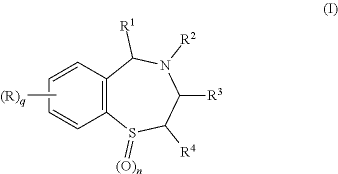
wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂) NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or —OH;
each R¹⁰ is —NR¹⁵R¹⁶, —OH, —SO₂R¹¹, —NHSO₂R¹¹, —C(═O)(R¹²), —NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, —OH, —NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
each R¹⁵ and R¹⁶ is independently acyl, alkenyl, alkoxyl, —OH, —NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
or a pharmaceutically-acceptable salt, hydrate, solvate, complex, or prodrug thereof.

15. The method of claim 13 or 14, wherein the calcium channel stabilizer is selected from the group consisting of: 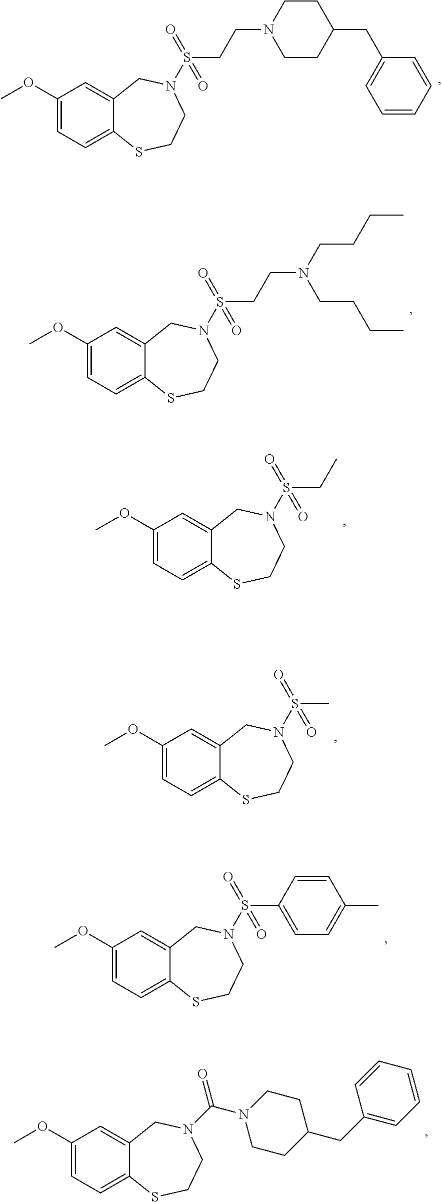 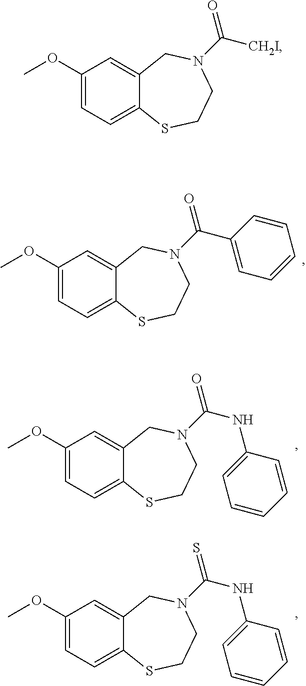 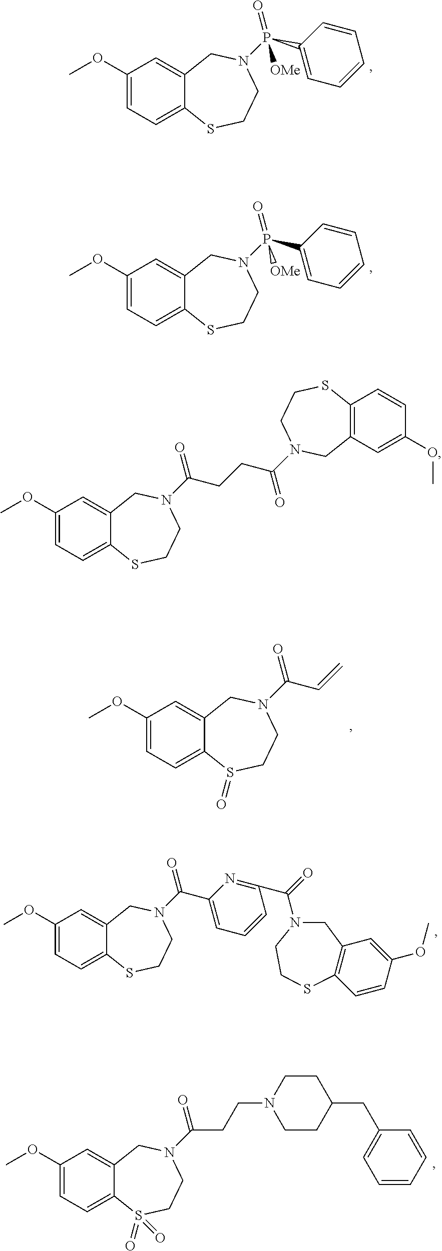 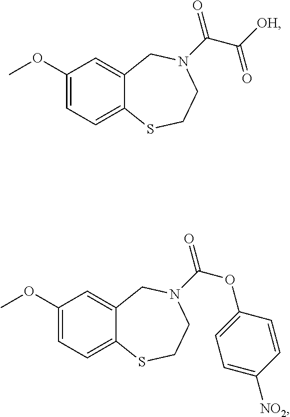 [SMILES: COc1ccc2c(c1)CN(C(=O)C(=O)O)CCS2 ; COc1ccc2c(c1)CN(C(=O)Oc1ccc([N+](=O)[O-])cc1)CCS2] 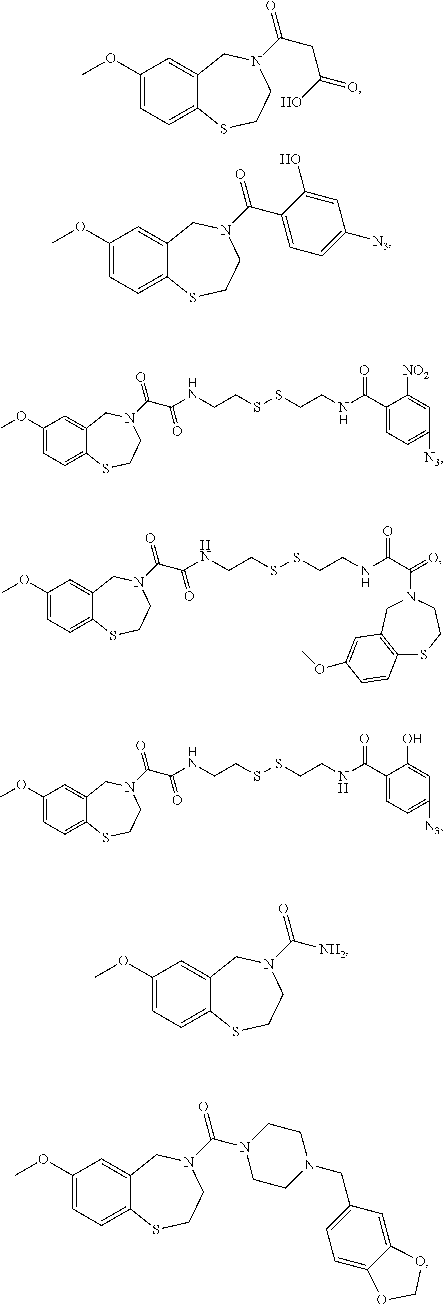 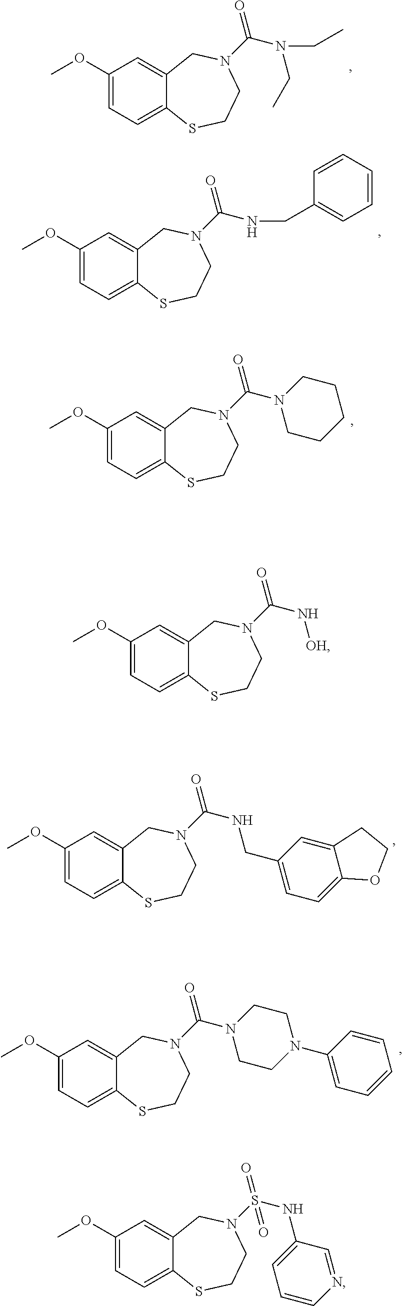 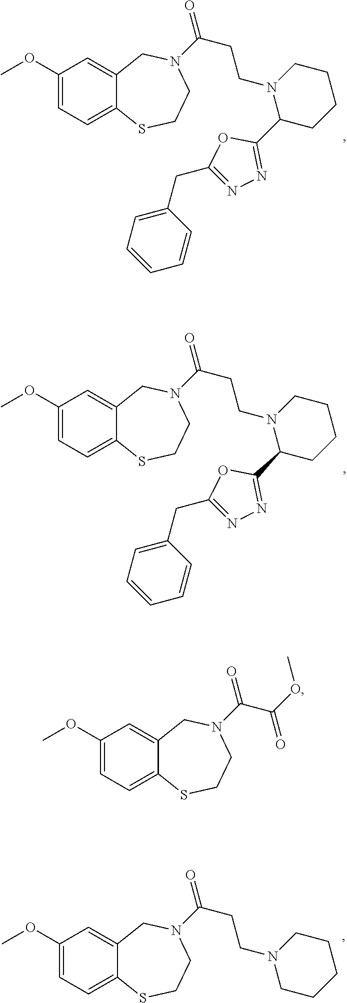 [SMILES: COc1ccc2c(c1)CN(C(=O)CCN1CCCCC1c1nnc(Cc3ccccc3)o1)CCS2 ; COc1ccc2c(c1)CN(C(=O)CCN1CCCC[C@H]1c1nnc(Cc3ccccc3)o1)CCS2 ; COC(=O)C(=O)N1CCSc2ccc(OC)cc2C1 ; COc1ccc2c(c1)CN(C(=O)CCN1CCCCC1)CCS2] 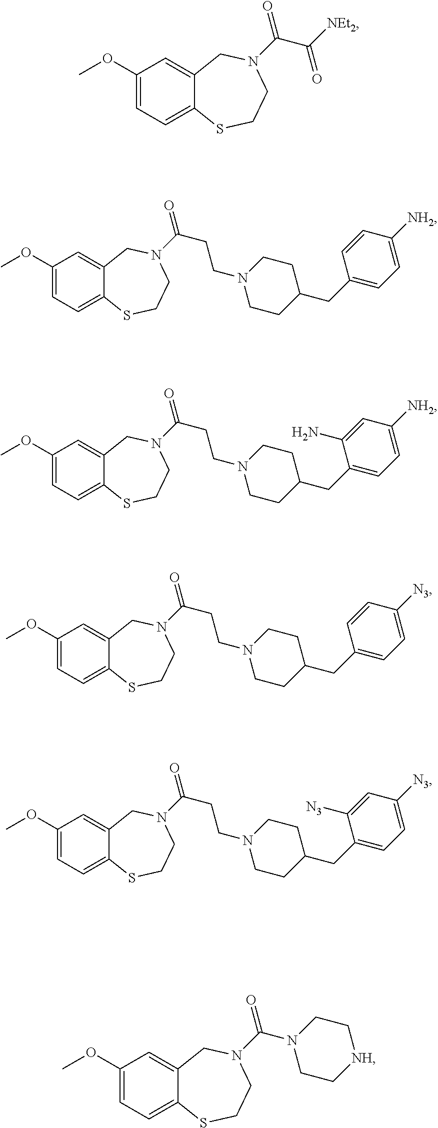 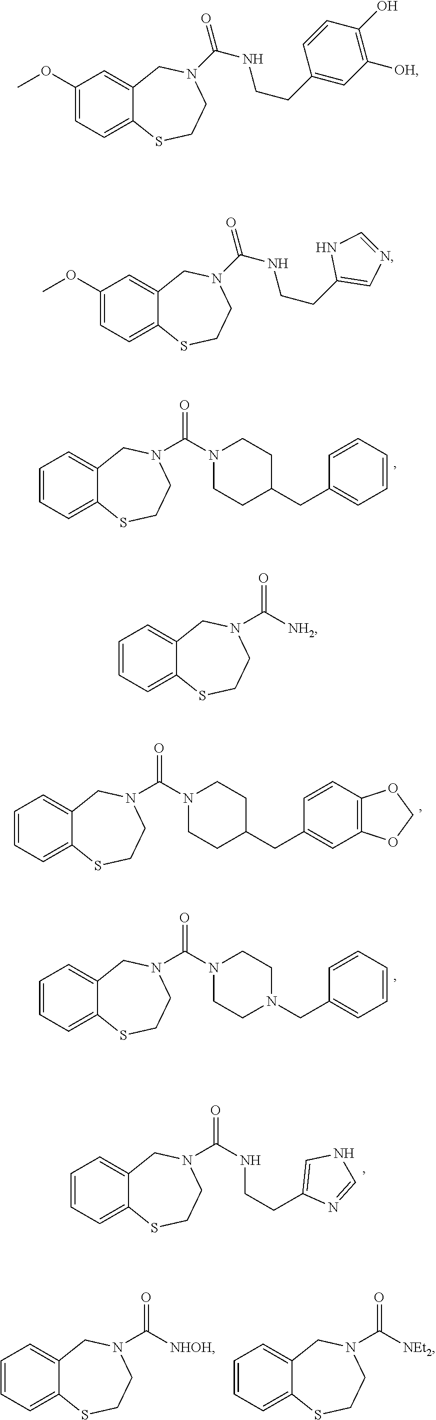 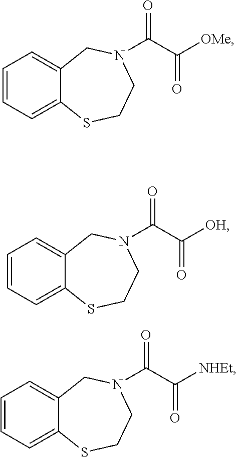 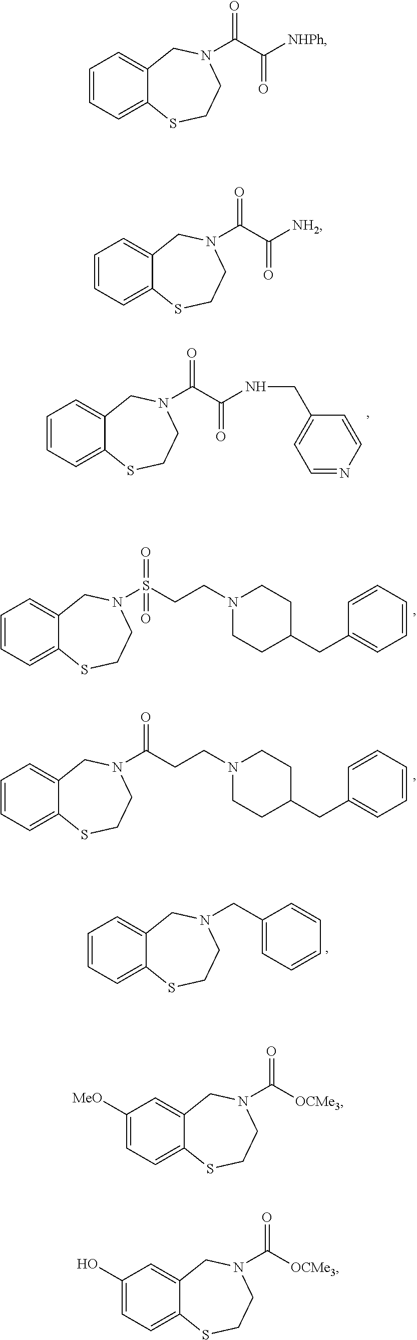 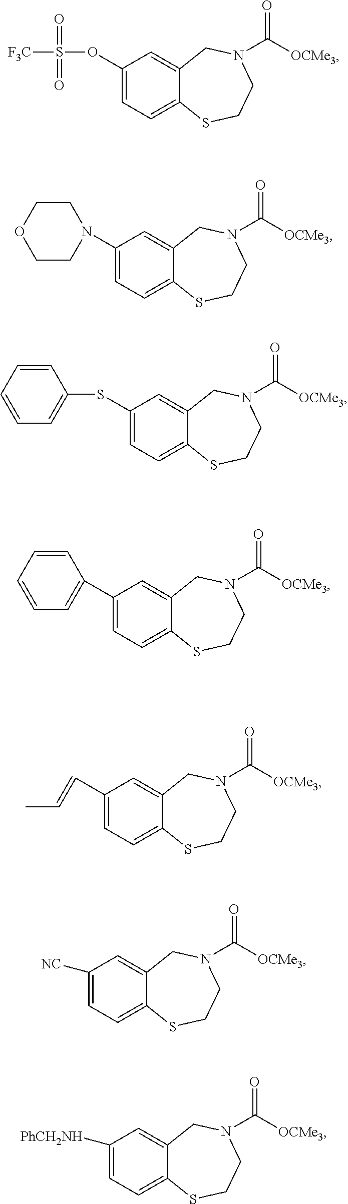 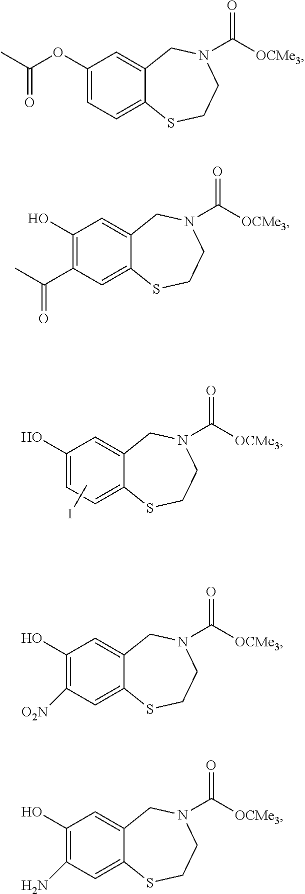 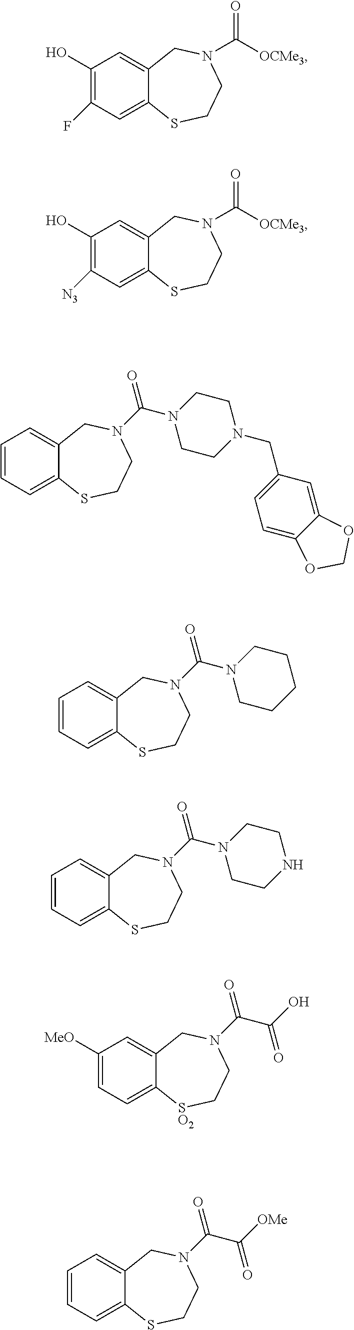 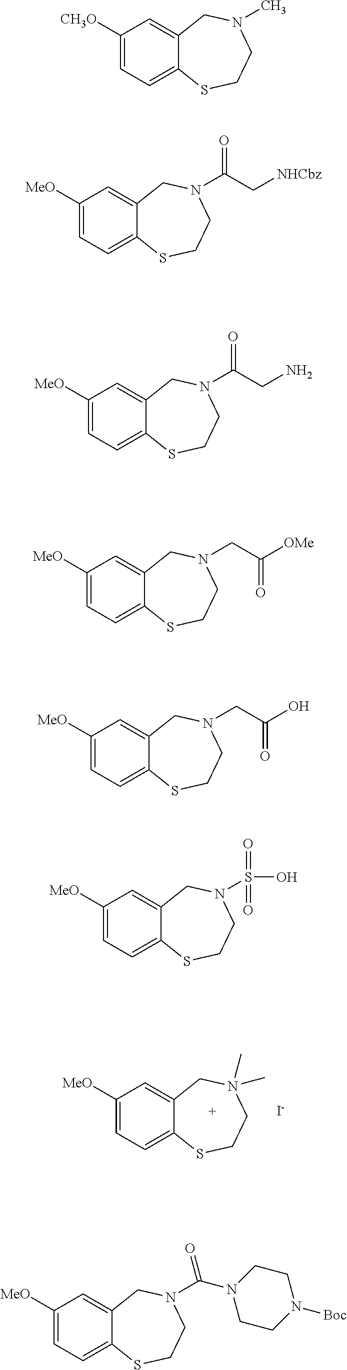 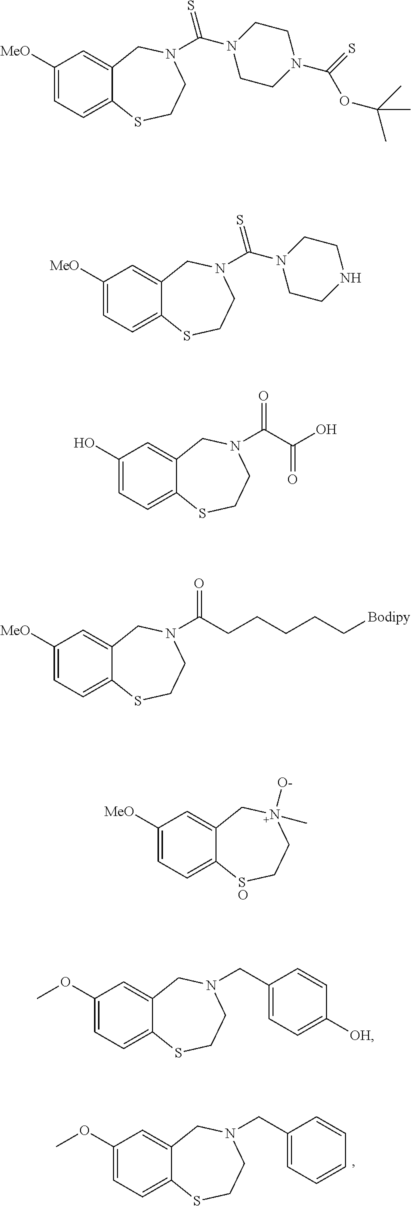 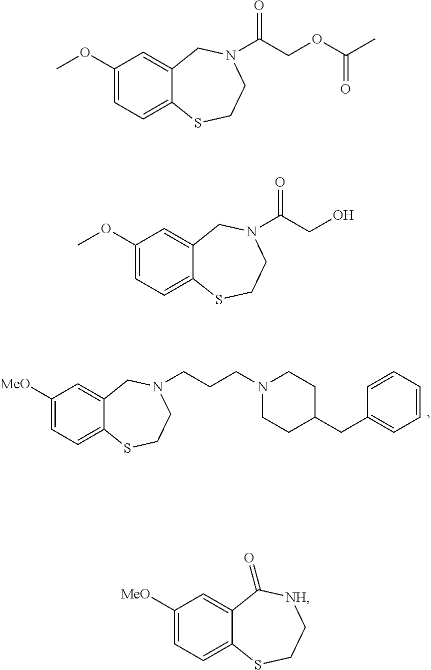 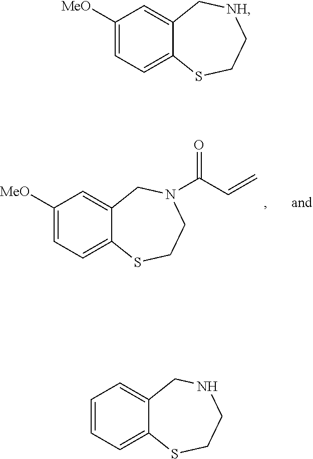 [SMILES: COc1ccc2c(c1)CNCCS2 ; C=CC(=O)N1CCSc2ccc(OC)cc2C1 ; c1ccc2c(c1)CNCCS2]
or a pharmaceutically-salt, hydrate, solvate, complex, or prodrug thereof.

16. The method of claim 1, wherein the calcium channel stabilizer is: 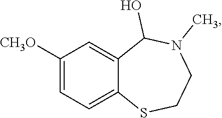
or a pharmaceutically-acceptable salt thereof.

17. The method of claim 1, wherein the calcium channel stabilizer is represented by the structural formula 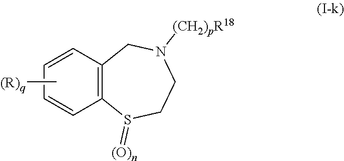
wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵,
q is 0, 1, 2, 3, or 4;
p is 1, 2, 3, 4, 5, 6, 7, 8 9, or 10; and
n is 0, 1, or 2,
or a pharmaceutically-acceptable salt thereof.

18. The method of claim 1, wherein the calcium channel stabilizer is able to penetrate the blood brain barrier.

19. The method of claim 1, wherein the calcium channel stabilizer is orally administered to the subject.

20. The method of claim 1, wherein the calcium channel stabilizer is administered in a pharmaceutical composition, the pharmaceutical composition further comprising at least one pharmaceutically-acceptable excipient.
